(19) United States
(12) Patent Application Publication
(10) Pub. No.: US 2022/0296633 A1
(43) Pub. Date: Sep. 22, 2022

(54) METHODS FOR TREATING MUSCULAR DYSTROPHY

(71) Applicant: Sarepta Therapeutics, Inc., Cambridge, MA (US)

(72) Inventor: Gilmore O'NEILL, Cambridge, MA (US)

(73) Assignee: Sarepta Therapeutics, Inc., Cambridge, MA (US)

(21) Appl. No.: 17/618,100

(22) PCT Filed: Jun. 18, 2020
(86) PCT No.: PCT/US2020/038483

§ 371 Date: Dec. 10, 2021

Related U.S. Application Data
(60) Provisional application No. 62/863,456, filed on Jun. 19, 2019.

(51) Int. Cl. A61K 31/7125; A61P 21/00; A61K 47/02; A61K 9/00; C12N 15/113

(52) U.S. Cl. CPC A61K 31/7125; A61P 21/00; A61K 47/02; A61K 9/0019; C12N 15/113; C12N 2310/11; C12N 2310/3145; C12N 2310/3233

(57) ABSTRACT

Methods for treating muscular dystrophy by administering a pharmaceutical composition comprising about 80 to about 300 mg/kg of an antisense oligomer, or pharmaceutically acceptable salt thereof, are described. In one embodiment, the compositions are administered less frequently than occurs in existing methods of treatment

### RELATED APPLICATIONS

This application is a 35 U.S.C. § 371 National Phase application of PCT/US2020/038483, filed Jun. 18, 2020, which claims the benefit of U.S. Provisional Application No. 62/863,456, filed on Jun. 19, 2019, the entire content of which is incorporated herein by reference.

### REFERENCE TO A SEQUENCE LISTING SUBMITTED ELECTRONICALLY VIA EFS-WEB

The content of the electronically submitted sequence listing is herein incorporated by reference into the specification in its entirety. The name of the text file containing the Sequence Listing is 4140_0320001_Seqlisting_ST25.txt. The size is 13,028 bytes and was created on Dec. 8, 2021 and is being submitted electronically via EFS-Web.

### FIELD OF THE DISCLOSURE

The disclosure relates to methods for treating muscular dystrophy in a human subject.

### BACKGROUND

Duchenne Muscular Dystrophy (DMD) is a serious, progressively debilitating, and ultimately fatal inherited X-linked neuromuscular disease. DMD is caused by mutations in the dystrophin gene characterized by the absence, or near absence, of functional dystrophin protein that disrupt the mRNA reading frame, resulting in a lack of dystrophin, a critically important part of the protein complex that connects the cytoskeletal actin of a muscle fiber to the extracellular matrix. In the absence of dystrophin, patients with DMD follow a predictable disease course. Affected patients, typically boys, develop muscle weakness in the first few years of life, lose the ability to walk during childhood, and usually require respiratory support by their late teens. Loss of functional abilities leads to loss of independence and increasing caregiver burden. Once lost, these abilities cannot be recovered. Despite improvements in the standard of care, such as the use of glucocorticoids, DMD remains an ultimately fatal disease, with patients usually dying of respiratory or cardiac failure in their mid to late 20s.

Progressive loss of muscle tissue and function in DMD is caused by the absence or near absence of functional dystrophin, a protein that plays a vital role in the structure and function of muscle cells. A potential therapeutic approach to the treatment of DMD is suggested by Becker muscular dystrophy (BMD), a milder dystrophinopathy. Both dystrophinopathies are caused by mutations of the DMD gene. In DMD, mutations that disrupt the pre-mRNA reading frame, referred to as “out-of-frame” mutations, prevent the production of functional dystrophin. In BMD, “in-frame” mutations do not disrupt the reading frame and result in the production of internally shortened, functional dystrophin protein.

An important approach for restoring these “out-of-frame” mutations is to utilize an antisense oligonucleotide to exclude or skip the molecular mutation of the DMD gene (dystrophin gene). The DMD or dystrophin gene is one of the largest genes in the human body and consists of 79 exons. Antisense oligonucleotides (AONs) have been specifically designed to target specific regions of the pre-mRNA, typically exons to induce the skipping of a mutation of the DMD gene thereby restoring these out-of-frame mutations in-frame to enable the production of internally shortened, yet functional dystrophin protein.

Exondys 51® (eteplirsen), is a phosphorodiamidate morpholino oligomer (PMO) designed to skip exon 51 of the human dystrophin gene in patients with DMD who are amenable to exon 51 skipping to restore the read frame and produce a functional shorter form of the dystrophin protein. The United States Food and Drug Administration (FDA) approved in 2016 EXONDYS 51® (eteplirsen, SEQ ID NO: 1) for the treatment of Duchenne muscular dystrophy (DMD) in patients who have a confirmed mutation of the DMD gene that is amenable to exon 51 skipping. The recommended dose of EXONDYS 51® is 30 mg/kg administered once weekly as a 35 to 60 minute intravenous infusion.

However there remains a need for improved methods for treating muscular dystrophy, such as DMD and BMD, in patients.

### SUMMARY

The present disclosure provides methods of treating DMD in a human subject having a mutation of the DMD gene that is amendable to exon skipping, comprising administering to the human subject a pharmaceutical composition comprising an antisense oligomer (e.g. eteplirsen), or pharmaceutically acceptable salt thereof, wherein the antisense oligomer, or pharmaceutically acceptable salt thereof, is administered at a dose of about 80 to about 300 mg/kg. In one embodiment of the method, eteplirsen, golodirsen, or casimersen is administered at a dose of about 80 to about 300 mg/kg. In one embodiment of the method, eteplirsen is administered at a dose of about 80 to about 300 mg/kg. In one embodiment of the method, golodirsen is administered at a dose of about 80 to about 300 mg/kg. In one embodiment of the method, casimersen is administered at a dose of about 80 to about 300 mg/kg.

In one embodiment, the present disclosure provides a method of treating DMD in a human subject having a mutation of the DMD gene that is amendable to exon 44, exon 45, exon 50, exon 51, exon 52, or exon 53 skipping, comprising administering to the human subject an antisense oligomer, or pharmaceutically acceptable salt thereof, wherein the antisense oligomer, or pharmaceutically acceptable salt thereof, is administered at a dose of about 100 mg/kg. In one embodiment of the method, eteplirsen, golodirsen, or casimersen is administered at a dose of about 100 mg/kg. In one embodiment of the method, eteplirsen is administered at a dose of about 100 mg/kg. In one embodiment of the method, golodirsen is administered at a dose of about 100 mg/kg. In one embodiment of the method, casimersen is administered at a dose of about 100 mg/kg.

In another embodiment, the present disclosure provides a method of treating DMD in a human subject having a mutation of the DMD gene that is amendable to exon 44, exon 45, exon 50, exon 51, exon 52, or exon 53 skipping, comprising administering to the human subject a pharmaceutical composition comprising an antisense oligomer, or pharmaceutically acceptable salt thereof, wherein the antisense oligomer, or pharmaceutically acceptable salt thereof, is administered at a dose of about 100 mg/kg. In one embodiment of the method, eteplirsen, golodirsen, or casimersen is administered at a dose of about 100 mg/kg. In one embodiment of the method, eteplirsen is administered at a dose of about 100 mg/kg. In one embodiment of the method, golodirsen is administered at a dose of about 100 mg/kg. In one embodiment of the method, casimersen is administered at a dose of about 100 mg/kg.

In one embodiment, the present disclosure provides a method of treating DMD in a human subject having a mutation of the DMD gene that is amendable to exon 44, exon 45, exon 50, exon 51, exon 52, or exon 53 skipping, comprising administering to the human subject a an antisense oligomer, or pharmaceutically acceptable salt thereof, wherein the antisense oligomer, or pharmaceutically acceptable salt thereof, is administered at a dose of about 200 mg/kg. In one embodiment of the method, eteplirsen, golodirsen, or casimersen is administered at a dose of about 200 mg/kg. In one embodiment of the method, eteplirsen is administered at a dose of about 200 mg/kg. In one embodiment of the method, golodirsen is administered at a dose of about 200 mg/kg. In one embodiment of the method, casimersen is administered at a dose of about 200 mg/kg.

In another embodiment, the present disclosure provides a method of treating DMD in a human subject having a mutation of the DMD gene that is amendable to exon 44, exon 45, exon 50, exon 51, exon 52, or exon 53 skipping, comprising administering to the human subject a pharmaceutical composition comprising an antisense oligomer, or pharmaceutically acceptable salt thereof, wherein the antisense oligomer, or pharmaceutically acceptable salt thereof, is administered at a dose of about 200 mg/kg. In one embodiment of the method, eteplirsen, golodirsen, or casimersen is administered at a dose of about 200 mg/kg. In one embodiment of the method, eteplirsen is administered at a dose of about 200 mg/kg. In one embodiment of the method, golodirsen is administered at a dose of about 200 mg/kg. In one embodiment of the method, casimersen is administered at a dose of about 200 mg/kg.

In one embodiment, the present disclosure provides a method of treating DMD in a human subject having a mutation of the DMD gene that is amendable to exon 44, exon 45, exon 50, exon 51, exon 52, or exon 53 skipping, comprising administering to the human subject a an antisense oligomer, or pharmaceutically acceptable salt thereof, wherein the antisense oligomer, or pharmaceutically acceptable salt thereof, is administered at a dose of about 300 mg/kg. In one embodiment of the method, eteplirsen, golodirsen, or casimersen is administered at a dose of about 300 mg/kg. In one embodiment of the method, eteplirsen is administered at a dose of about 300 mg/kg. In one embodiment of the method, golodirsen is administered at a dose of about 300 mg/kg. In one embodiment of the method, casimersen is administered at a dose of about 300 mg/kg.

In another embodiment, the present disclosure provides a method of treating DMD in a human subject having a mutation of the DMD gene that is amendable to exon 44, exon 45, exon 50, exon 51, exon 52, or exon 53 skipping, comprising administering to the human subject a pharmaceutical composition comprising an antisense oligomer, or pharmaceutically acceptable salt thereof, wherein the antisense oligomer, or pharmaceutically acceptable salt thereof, is administered at a dose of about 300 mg/kg. In one embodiment of the method, eteplirsen, golodirsen, or casimersen is administered at a dose of about 300 mg/kg. In one embodiment of the method, eteplirsen is administered at a dose of about 300 mg/kg. In one embodiment of the method, golodirsen is administered at a dose of about 300 mg/kg. In one embodiment of the method, casimersen is administered at a dose of about 300 mg/kg.

The disclosure also relates to methods of restoring an mRNA reading frame to induce dystrophin production in a human subject having a mutation of the dystrophin gene that is amenable to exon 44, exon 45, exon 50, exon 51, exon 52, or exon 53 skipping, the method comprising administering to the subject a pharmaceutical composition comprising an antisense oligomer, or pharmaceutically acceptable salt thereof, wherein the antisense oligomer, or pharmaceutically acceptable salt thereof, is administered at a dose of about 80 to about 300 mg/kg. In one embodiment of the method, eteplirsen, golodirsen, or casimersen is administered at a dose of about 80 to about 300 mg/kg. In one embodiment of the method, eteplirsen is administered at a dose of about 80 to about 300 mg/kg. In one embodiment of the method, golodirsen is administered at a dose of about 80 to about 300 mg/kg. In one embodiment of the method, casimersen is administered at a dose of about 80 to about 300 mg/kg.

In one embodiment, the disclosure provides a method of restoring an mRNA reading frame to induce dystrophin production in a human subject having a mutation of the dystrophin gene that is amenable to exon 44, exon 45, exon 50, exon 51, exon 52, or exon 53 skipping, the method comprising administering to the subject a pharmaceutical composition comprising an antisense oligomer, or pharmaceutically acceptable salt thereof, wherein the antisense oligomer, or pharmaceutically acceptable salt thereof, is administered at a dose of about 100 mg/kg. In one embodiment of the method, eteplirsen, golodirsen, or casimersen is administered at a dose of about 100 mg/kg. In one embodiment of the method, eteplirsen is administered at a dose of about 100 mg/kg. In one embodiment of the method, golodirsen is administered at a dose of about 100 mg/kg. In one embodiment of the method, casimersen is administered at a dose of about 100 mg/kg.

In another embodiment, the disclosure provides a method of restoring an mRNA reading frame to induce dystrophin production in a human subject having a mutation of the dystrophin gene that is amenable to exon 44, exon 45, exon 50, exon 51, exon 52, or exon 53 skipping, the method comprising administering to the subject a pharmaceutical composition comprising an antisense oligomer, or pharmaceutically acceptable salt thereof, wherein the antisense oligomer, or pharmaceutically acceptable salt thereof, is administered at a dose of about 200 mg/kg. In one embodiment of the method, eteplirsen, golodirsen, or casimersen is administered at a dose of about 200 mg/kg. In one embodiment of the method, eteplirsen is administered at a dose of about 200 mg/kg. In one embodiment of the method, golodirsen is administered at a dose of about 200 mg/kg. In one embodiment of the method, casimersen is administered at a dose of about 200 mg/kg.

In one embodiment, the disclosure provides a method of restoring an mRNA reading frame to induce dystrophin production in a human subject having a mutation of the dystrophin gene that is amenable to exon 44, exon 45, exon 50, exon 51, exon 52, or exon 53 skipping, the method comprising administering to the subject a pharmaceutical composition comprising an antisense oligomer, or pharmaceutically acceptable salt thereof, wherein the antisense oligomer, or pharmaceutically acceptable salt thereof, is administered at a dose of about 300 mg/kg. In one embodiment of the method, eteplirsen, golodirsen, or casimersen is administered at a dose of about 300 mg/kg. In one embodiment of the method, eteplirsen is administered at a dose of about 300 mg/kg. In one embodiment of the method, golodirsen is administered at a dose of about 300 mg/kg. In one embodiment of the method, casimersen is administered at a dose of about 300 mg/kg.

The present disclosure also relates a method of excluding 44, exon 45, exon 50, exon 51, exon 52, or exon 53 from dystrophin pre-mRNA during mRNA processing in a human subject having a mutation of the dystrophin gene that is amenable to 44, exon 45, exon 50, exon 51, exon 52, or exon 53 skipping, the method comprising administering to the subject a pharmaceutical composition comprising an antisense oligomer, or pharmaceutically acceptable salt thereof, wherein the antisense oligomer, or pharmaceutically acceptable salt thereof, is administered at a dose of about 80 to about 300 mg/kg. In one embodiment of the method, eteplirsen, golodirsen, or casimersen is administered at a dose of about 80 to about 300 mg/kg. In one embodiment of the method, eteplirsen is administered at a dose of about 80 to about 300 mg/kg. In one embodiment of the method, golodirsen is administered at a dose of about 80 to about 300 mg/kg. In one embodiment of the method, casimersen is administered at a dose of about 80 to about 300 mg/kg.

In one embodiment, the disclosure provides a method of excluding exon 44, exon 45, exon 50, exon 51, exon 52, or exon 53 from dystrophin pre-mRNA during mRNA processing in a human subject having a mutation of the dystrophin gene that is amenable to exon 44, exon 45, exon 50, exon 51, exon 52, or exon 53 skipping, the method comprising administering to the subject a pharmaceutical composition comprising an antisense oligomer, or pharmaceutically acceptable salt thereof, wherein the antisense oligomer, or pharmaceutically acceptable salt thereof, is administered at a dose of about 100 mg/kg. In one embodiment of the method, eteplirsen, golodirsen, or casimersen is administered at a dose of about 100 mg/kg. In one embodiment of the method, eteplirsen is administered at a dose of about 100 mg/kg. In one embodiment of the method, golodirsen is administered at a dose of about 100 mg/kg. In one embodiment of the method, casimersen is administered at a dose of about 100 mg/kg.

In another embodiment, the disclosure provides a method of excluding exon 44, exon 45, exon 50, exon 51, exon 52, or exon 53 from dystrophin pre-mRNA during mRNA processing in a human subject having a mutation of the dystrophin gene that is amenable to exon 44, exon 45, exon 50, exon 51, exon 52, or exon 53 skipping, the method comprising administering to the subject a pharmaceutical composition comprising an antisense oligomer, or pharmaceutically acceptable salt thereof, wherein the antisense oligomer, or pharmaceutically acceptable salt thereof, is administered at a dose of about 200 mg/kg. In one embodiment of the method, eteplirsen, golodirsen, or casimersen is administered at a dose of about 200 mg/kg. In one embodiment of the method, eteplirsen is administered at a dose of about 200 mg/kg. In one embodiment of the method, golodirsen is administered at a dose of about 200 mg/kg. In one embodiment of the method, casimersen is administered at a dose of about 200 mg/kg.

In another embodiment, the disclosure provides a method of excluding exon 44, exon 45, exon 50, exon 51, exon 52, or exon 53 from dystrophin pre-mRNA during mRNA processing in a human subject having a mutation of the dystrophin gene that is amenable to exon 44, exon 45, exon 50, exon 51, exon 52, or exon 53 skipping, the method comprising administering to the subject a pharmaceutical composition comprising an antisense oligomer, or pharmaceutically acceptable salt thereof, wherein the antisense oligomer, or pharmaceutically acceptable salt thereof, is administered at a dose of about 300 mg/kg. In one embodiment of the method, eteplirsen, golodirsen, or casimersen is administered at a dose of about 300 mg/kg. In one embodiment of the method, eteplirsen is administered at a dose of about 300 mg/kg. In one embodiment of the method, golodirsen is administered at a dose of about 300 mg/kg. In one embodiment of the method, casimersen is administered at a dose of about 200 mg/kg.

In another aspect, the disclosure provides a method of binding exon 44, exon 45, exon 50, exon 51, exon 52, or exon 53 of dystrophin pre-mRNA in a human subject having a mutation of the dystrophin gene that is amenable to exon 44, exon 45, exon 50, exon 51, exon 52, or exon 53 skipping, the method comprising administering to the subject a pharmaceutical composition comprising an antisense oligomer, or pharmaceutically acceptable salt thereof, wherein the antisense oligomer, or pharmaceutically acceptable salt thereof, is administered at a dose of about 80 to about 300 mg/kg. In one embodiment of the method, eteplirsen, golodirsen, or casimersen is administered at a dose of about 80 to about 300 mg/kg. In one embodiment of the method, eteplirsen is administered at a dose of about 80 to about 300 mg/kg. In one embodiment of the method, golodirsen is administered at a dose of about 80 to about 300 mg/kg. In one embodiment of the method, casimersen is administered at a dose of about 80 to about 300 mg/kg.

In one embodiment, the disclosure provides a method of binding exon 44, exon 45, exon 50, exon 51, exon 52, or exon 53 of dystrophin pre-mRNA in a human subject having a mutation of the dystrophin gene that is amenable to exon 44, exon 45, exon 50, exon 51, exon 52, or exon 53 skipping, the method comprising administering to the subject a pharmaceutical composition comprising an antisense oligomer, or pharmaceutically acceptable salt thereof, wherein an antisense oligomer, or pharmaceutically acceptable salt thereof, is administered at a dose of about 100 mg/kg. In one embodiment of the method, eteplirsen, golodirsen, or casimersen is administered at a dose of about 100 mg/kg. In one embodiment of the method, eteplirsen is administered at a dose of about 100 mg/kg. In one embodiment of the method, golodirsen is administered at a dose of about 100 mg/kg. In one embodiment of the method, casimersen is administered at a dose of about 100 mg/kg.

In one embodiment, the disclosure provides a method of binding exon 44, exon 45, exon 50, exon 51, exon 52, or exon 53 of dystrophin pre-mRNA in a human subject having a mutation of the dystrophin gene that is amenable to exon 44, exon 45, exon 50, exon 51, exon 52, or exon 53 skipping, the method comprising administering to the subject a pharmaceutical composition comprising an antisense oligomer, or pharmaceutically acceptable salt thereof, wherein the antisense oligomer, or pharmaceutically acceptable salt thereof, is administered at a dose of about 200 mg/kg. In one embodiment of the method, eteplirsen, golodirsen, or casimersen is administered at a dose of about 200 mg/kg. In one embodiment of the method, eteplirsen is administered at a dose of about 200 mg/kg. In one embodiment of the method, golodirsen is administered at a dose of about 200 mg/kg. In one embodiment of the method, casimersen is administered at a dose of about 200 mg/kg.

In one embodiment, the disclosure provides a method of binding exon 44, exon 45, exon 50, exon 51, exon 52, or exon 53 of dystrophin pre-mRNA in a human subject having a mutation of the dystrophin gene that is amenable to exon 44, exon 45, exon 50, exon 51, exon 52, or exon 53 skipping, the method comprising administering to the subject a pharmaceutical composition comprising an antisense oligomer, or pharmaceutically acceptable salt thereof, wherein the antisense oligomer, or pharmaceutically acceptable salt thereof, is administered at a dose of about 300 mg/kg. In one embodiment of the method, eteplirsen, golodirsen, or casimersen is administered at a dose of about 300 mg/kg. In one embodiment of the method, eteplirsen is administered at a dose of about 300 mg/kg. In one embodiment of the method, golodirsen is administered at a dose of about 300 mg/kg. In one embodiment of the method, casimersen is administered at a dose of about 300 mg/kg.

In certain embodiments, the methods of the present disclosure comprise administering to a human subject a pharmaceutical composition comprising an antisense oligomer (e.g., eteplirsen), or pharmaceutically acceptable salt thereof, wherein the antisense oligomer, or pharmaceutically acceptable salt thereof, is administered at a dose of about 80 to about 300 mg/kg. In some embodiments, an antisense oligomer (e.g., eteplirsen), or pharmaceutically acceptable salt thereof, is administered at a dose of about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg. In some embodiments, an antisense oligomer (e.g., golodirsen), or pharmaceutically acceptable salt thereof, is administered at a dose of about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg. In some embodiments, an antisense oligomer (e.g., casimersen), or pharmaceutically acceptable salt thereof, is administered at a dose of about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg. In some embodiments, an antisense oligomer, or pharmaceutically acceptable salt thereof, (e.g., eteplirsen) is administered at a dose of about 100 mg/kg. In other embodiments, an antisense oligomer (e.g., eteplirsen), or pharmaceutically acceptable salt thereof, is administered at a dose of about 200 mg/kg.

In some embodiments, the methods of the present disclosure comprise administering a pharmaceutical composition comprising an antisense oligomer (e.g., eteplirsen), or pharmaceutically acceptable salt thereof, wherein the pharmaceutical composition is administered once weekly. In one embodiment, a method comprises administering to the human subject a pharmaceutical composition comprising an antisense oligomer (e.g., eteplirsen), or pharmaceutically acceptable salt thereof, wherein the antisense oligomer, or pharmaceutically acceptable salt thereof, is administered at a dose of about 100 mg/kg, wherein the pharmaceutical composition is administered once weekly. In another embodiment, a method comprises administering to the human subject a pharmaceutical composition comprising an antisense oligomer (e.g., golodirsen), or pharmaceutically acceptable salt thereof, wherein the antisense oligomer, or pharmaceutically acceptable salt thereof, is administered at a dose of about 100 mg/kg, wherein the pharmaceutical composition is administered once weekly. In one embodiment, a method comprises administering to the human subject a pharmaceutical composition comprising an antisense oligomer (e.g., casimeren), or pharmaceutically acceptable salt thereof, wherein the antisense oligomer, or pharmaceutically acceptable salt thereof, is administered at a dose of about 100 mg/kg, wherein the pharmaceutical composition is administered once weekly. In another embodiment, a method comprises administering to the human subject a pharmaceutical composition comprising an antisense oligomer (e.g., eteplirsen), or pharmaceutically acceptable salt thereof, wherein the antisense oligomer, or pharmaceutically acceptable salt thereof, is administered at a dose of about 100 mg/kg.

In some embodiments, the methods of the present disclosure comprise administering a pharmaceutical composition comprising an antisense oligomer (e.g., eteplirsen), or pharmaceutically acceptable salt thereof, wherein the pharmaceutical composition is administered once weekly. In one embodiment, a method comprises administering to the human subject a pharmaceutical composition comprising an antisense oligomer (e.g., eteplirsen), or pharmaceutically acceptable salt thereof, wherein the antisense oligomer, or pharmaceutically acceptable salt thereof, is administered at a dose of about 200 mg/kg, wherein the pharmaceutical composition is administered once weekly. In another embodiment, a method comprises administering to the human subject a pharmaceutical composition comprising an antisense oligomer (e.g., golodirsen), or pharmaceutically acceptable salt thereof, wherein the antisense oligomer, or pharmaceutically acceptable salt thereof, is administered at a dose of about 200 mg/kg, wherein the pharmaceutical composition is administered once weekly. In one embodiment, a method comprises administering to the human subject a pharmaceutical composition comprising an antisense oligomer (e.g., casimeren), or pharmaceutically acceptable salt thereof, wherein the antisense oligomer, or pharmaceutically acceptable salt thereof, is administered at a dose of about 200 mg/kg, wherein the pharmaceutical composition is administered once weekly. In another embodiment, a method comprises administering to the human subject a pharmaceutical composition comprising an antisense oligomer (e.g., eteplirsen), or pharmaceutically acceptable salt thereof, wherein the antisense oligomer, or pharmaceutically acceptable salt thereof, is administered at a dose of about 200 mg/kg.

In some embodiments, the methods of the present disclosure comprise administering a pharmaceutical composition comprising an antisense oligomer (e.g., eteplirsen), or pharmaceutically acceptable salt thereof, wherein the pharmaceutical composition is administered once weekly. In one embodiment, a method comprises administering to the human subject a pharmaceutical composition comprising an antisense oligomer (e.g., eteplirsen), or pharmaceutically acceptable salt thereof, wherein the antisense oligomer, or pharmaceutically acceptable salt thereof, is administered at a dose of about 300 mg/kg, wherein the pharmaceutical composition is administered once weekly. In another embodiment, a method comprises administering to the human subject a pharmaceutical composition comprising an antisense oligomer (e.g., golodirsen), or pharmaceutically acceptable salt thereof, wherein the antisense oligomer, or pharmaceutically acceptable salt thereof, is administered at a dose of about 300 mg/kg, wherein the pharmaceutical composition is administered once weekly. In one embodiment, a method comprises administering to the human subject a pharmaceutical composition comprising an antisense oligomer (e.g., casimeren), or pharmaceutically acceptable salt thereof, wherein the antisense oligomer, or pharmaceutically acceptable salt thereof, is administered at a dose of about 300 mg/kg, wherein the pharmaceutical composition is administered once weekly. In another embodiment, a method comprises administering to the human subject a pharmaceutical composition comprising an antisense oligomer (e.g., eteplirsen), or pharmaceutically acceptable salt thereof, wherein the antisense oligomer, or pharmaceutically acceptable salt thereof, is administered at a dose of about 300 mg/kg.

In some embodiments, the methods of the present disclosure comprise administering a pharmaceutical composition comprising an antisense oligomer (e.g., eteplirsen), or pharmaceutically acceptable salt thereof, for up to about 24 weeks, up to about 48 weeks, up to about 60 weeks, up to about 80 weeks, up to about 100 weeks, up to about 120 weeks, up to about 140 weeks, up to about 150 weeks, up to about 160 weeks, up to about 180 weeks, or up to about 200 weeks. In some embodiments, the pharmaceutical composition comprising an antisense oligomer (e.g., eteplirsen), or pharmaceutically acceptable salt thereof, is administered for up to about 144 weeks. In some embodiments, the composition comprising an antisense oligomer (e.g., eteplirsen), or pharmaceutically acceptable salt thereof, is administered for at least 24 weeks, at least 36 weeks, at least 48 weeks, at least 120 weeks, at least 144 weeks, or at least 164 weeks. In yet other embodiments, the pharmaceutical composition comprising an antisense oligomer (e.g., eteplirsen), or pharmaceutically acceptable salt thereof, is administered for the duration of the illness.

In one embodiment, a method comprises administering to the human subject a pharmaceutical composition comprising an antisense oligomer (e.g., eteplirsen), or pharmaceutically acceptable salt thereof, for up to about 24 weeks, wherein the antisense oligomer (e.g., eteplirsen), or pharmaceutically acceptable salt thereof, is administered at a dose of about 100 mg/kg. In another embodiment, a method comprises administering to the human subject a pharmaceutical composition comprising an antisense oligomer (e.g., eteplirsen), or pharmaceutically acceptable salt thereof, for up to about 48 weeks, wherein the antisense oligomer, or pharmaceutically acceptable salt thereof, is administered at a dose of about 100 mg/kg. In yet another embodiment, a method comprises administering to the human subject a pharmaceutical composition comprising an antisense oligomer (e.g., eteplirsen), or pharmaceutically acceptable salt thereof, for up to about 144 weeks, wherein the antisense oligomer, or pharmaceutically acceptable salt thereof, is administered at a dose of about 100 mg/kg.

In one embodiment, a method comprises administering to the human subject a pharmaceutical composition comprising an antisense oligomer (e.g., eteplirsen), or pharmaceutically acceptable salt thereof, for up to about 24 weeks, wherein the antisense oligomer, or pharmaceutically acceptable salt thereof, is administered at a dose of about 200 mg/kg. In another embodiment, a method comprises administering to the human subject a pharmaceutical composition comprising an antisense oligomer (e.g., eteplirsen) or pharmaceutically acceptable salt thereof, for up to about 48 weeks, wherein the antisense oligomer (e.g., eteplirsen), or pharmaceutically acceptable salt thereof, is administered at a dose of about 200 mg/kg. In yet another embodiment, a method comprises administering to the human subject a pharmaceutical composition comprising an antisense oligomer (e.g., eteplirsen), or pharmaceutically acceptable salt thereof, for up to about 144 weeks, wherein the antisense oligomer, or pharmaceutically acceptable salt thereof, is administered at a dose of about 200 mg/kg.

In one embodiment, a method comprises administering to the human subject a pharmaceutical composition comprising an antisense oligomer (e.g., eteplirsen), or pharmaceutically acceptable salt thereof, for up to about 24 weeks, wherein the antisense oligomer, or pharmaceutically acceptable salt thereof, is administered at a dose of about 300 mg/kg. In another embodiment, a method comprises administering to the human subject a pharmaceutical composition comprising an antisense oligomer (e.g., eteplirsen) or pharmaceutically acceptable salt thereof, for up to about 48 weeks, wherein the antisense oligomer (e.g., eteplirsen), or pharmaceutically acceptable salt thereof, is administered at a dose of about 300 mg/kg. In yet another embodiment, a method comprises administering to the human subject a pharmaceutical composition comprising an antisense oligomer (e.g., eteplirsen), or pharmaceutically acceptable salt thereof, for up to about 144 weeks, wherein the antisense oligomer, or pharmaceutically acceptable salt thereof, is administered at a dose of about 300 mg/kg.

In one embodiment, a method comprises administering to the human subject a pharmaceutical composition comprising an antisense oligomer (e.g., golodirsen), or pharmaceutically acceptable salt thereof, for up to about 24 weeks, wherein the antisense oligomer, or pharmaceutically acceptable salt thereof, is administered at a dose of about 100 mg/kg. In another embodiment, a method comprises administering to the human subject a pharmaceutical composition comprising an antisense oligomer (e.g., golodirsen) or pharmaceutically acceptable salt thereof, for up to about 48 weeks, wherein the antisense oligomer (e.g., golodirsen), or pharmaceutically acceptable salt thereof, is administered at a dose of about 100 mg/kg. In yet another embodiment, a method comprises administering to the human subject a pharmaceutical composition comprising an antisense oligomer (e.g., golodirsen), or pharmaceutically acceptable salt thereof, for up to about 144 weeks, wherein the antisense oligomer, or pharmaceutically acceptable salt thereof, is administered at a dose of about 100 mg/kg.

In one embodiment, a method comprises administering to the human subject a pharmaceutical composition comprising an antisense oligomer (e.g., golodirsen), or pharmaceutically acceptable salt thereof, for up to about 24 weeks, wherein the antisense oligomer, or pharmaceutically acceptable salt thereof, is administered at a dose of about 200 mg/kg. In another embodiment, a method comprises administering to the human subject a pharmaceutical composition comprising an antisense oligomer (e.g., golodirsen) or pharmaceutically acceptable salt thereof, for up to about 48 weeks, wherein the antisense oligomer (e.g., golodirsen), or pharmaceutically acceptable salt thereof, is administered at a dose of about 200 mg/kg. In yet another embodiment, a method comprises administering to the human subject a pharmaceutical composition comprising an antisense oligomer (e.g., golodirsen), or pharmaceutically acceptable salt thereof, for up to about 144 weeks, wherein the antisense oligomer, or pharmaceutically acceptable salt thereof, is administered at a dose of about 200 mg/kg.

In one embodiment, a method comprises administering to the human subject a pharmaceutical composition comprising an antisense oligomer (e.g., golodirsen), or pharmaceutically acceptable salt thereof, for up to about 24 weeks, wherein the antisense oligomer, or pharmaceutically acceptable salt thereof, is administered at a dose of about 300 mg/kg. In another embodiment, a method comprises administering to the human subject a pharmaceutical composition comprising an antisense oligomer (e.g., golodirsen) or pharmaceutically acceptable salt thereof, for up to about 48 weeks, wherein the antisense oligomer (e.g., golodirsen), or pharmaceutically acceptable salt thereof, is administered at a dose of about 300 mg/kg. In yet another embodiment, a method comprises administering to the human subject a pharmaceutical composition comprising an antisense oligomer (e.g., golodirsen), or pharmaceutically acceptable salt thereof, for up to about 144 weeks, wherein the antisense oligomer, or pharmaceutically acceptable salt thereof, is administered at a dose of about 300 mg/kg.

In one embodiment, a method comprises administering to the human subject a pharmaceutical composition comprising an antisense oligomer (e.g., casimersen), or pharmaceutically acceptable salt thereof, for up to about 24 weeks, wherein the antisense oligomer, or pharmaceutically acceptable salt thereof, is administered at a dose of about 100 mg/kg. In another embodiment, a method comprises administering to the human subject a pharmaceutical composition comprising an antisense oligomer (e.g., casimersen) or pharmaceutically acceptable salt thereof, for up to about 48 weeks, wherein the antisense oligomer (e.g., casimersen), or pharmaceutically acceptable salt thereof, is administered at a dose of about 100 mg/kg. In yet another embodiment, a method comprises administering to the human subject a pharmaceutical composition comprising an antisense oligomer (e.g., casimersen), or pharmaceutically acceptable salt thereof, for up to about 144 weeks, wherein the antisense oligomer, or pharmaceutically acceptable salt thereof, is administered at a dose of about 100 mg/kg.

In one embodiment, a method comprises administering to the human subject a pharmaceutical composition comprising an antisense oligomer (e.g., casimersen), or pharmaceutically acceptable salt thereof, for up to about 24 weeks, wherein the antisense oligomer, or pharmaceutically acceptable salt thereof, is administered at a dose of about 200 mg/kg. In another embodiment, a method comprises administering to the human subject a pharmaceutical composition comprising an antisense oligomer (e.g., casimersen) or pharmaceutically acceptable salt thereof, for up to about 48 weeks, wherein the antisense oligomer (e.g., casimersen), or pharmaceutically acceptable salt thereof, is administered at a dose of about 200 mg/kg. In yet another embodiment, a method comprises administering to the human subject a pharmaceutical composition comprising an antisense oligomer (e.g., casimersen), or pharmaceutically acceptable salt thereof, for up to about 144 weeks, wherein the antisense oligomer, or pharmaceutically acceptable salt thereof, is administered at a dose of about 200 mg/kg.

In one embodiment, a method comprises administering to the human subject a pharmaceutical composition comprising an antisense oligomer (e.g., casimersen), or pharmaceutically acceptable salt thereof, for up to about 24 weeks, wherein the antisense oligomer, or pharmaceutically acceptable salt thereof, is administered at a dose of about 300 mg/kg. In another embodiment, a method comprises administering to the human subject a pharmaceutical composition comprising an antisense oligomer (e.g., casimersen) or pharmaceutically acceptable salt thereof, for up to about 48 weeks, wherein the antisense oligomer (e.g., casimersen), or pharmaceutically acceptable salt thereof, is administered at a dose of about 300 mg/kg. In yet another embodiment, a method comprises administering to the human subject a pharmaceutical composition comprising an antisense oligomer (e.g., casimersen), or pharmaceutically acceptable salt thereof, for up to about 144 weeks, wherein the anti sense oligomer, or pharmaceutically acceptable salt thereof, is administered at a dose of about 300 mg/kg.

In some embodiments, the pharmaceutical composition comprising an antisense oligomer (e.g., eteplirsen), or pharmaceutically acceptable salt thereof, is formulated for systemic administration. In some embodiments, the composition comprising an antisense oligomer (e.g., eteplirsen), or pharmaceutically acceptable salt thereof, is administered intravenously. In some embodiments, the composition comprising an antisense oligomer (e.g., eteplirsen), or pharmaceutically acceptable salt thereof, is administered as an intravenous infusion. In some embodiments, the pharmaceutical composition comprising an antisense oligomer (e.g., eteplirsen), or pharmaceutically acceptable salt thereof, is administered as an intravenous infusion over 35 to 60 minutes. In other embodiments, the composition comprising an antisense oligomer (e.g., eteplirsen), or pharmaceutically acceptable salt thereof, is administered intramuscularly. It yet other embodiments, the composition comprising an antisense oligomer (e.g., eteplirsen) or pharmaceutically acceptable salt thereof, is administered orally.

In some embodiments, the methods of the present disclosure comprise administering the pharmaceutical composition comprising an antisense oligomer (e.g., eteplirsen), or pharmaceutically acceptable salt thereof, to a male human subject, wherein the antisense oligomer, or pharmaceutically acceptable salt thereof, is administered at a dose of about 80 to about 300 mg/kg. In one embodiment, an antisense oligomer (e.g., eteplirsen), or pharmaceutically acceptable salt thereof, is administered at a dose of about 100 mg/kg. In another embodiment, an antisense oligomer (e.g., eteplirsen), or pharmaceutically acceptable salt thereof, is administered at a dose of 200 mg/kg. In another embodiment, an antisense oligomer (e.g., eteplirsen), or pharmaceutically acceptable salt thereof, is administered at a dose of 300 mg/kg. In some embodiments, the human subject is 7 to 13 years of age (inclusive).

In some embodiments, the methods of the present disclosure comprise administering the pharmaceutical composition comprising an antisense oligomer (e.g., golodirsen), or pharmaceutically acceptable salt thereof, to a male human subject, wherein the antisense oligomer, or pharmaceutically acceptable salt thereof, is administered at a dose of about 80 to about 300 mg/kg. In one embodiment, an antisense oligomer (e.g., golodirsen), or pharmaceutically acceptable salt thereof, is administered at a dose of about 100 mg/kg. In another embodiment, an antisense oligomer (e.g., golodirsen), or pharmaceutically acceptable salt thereof, is administered at a dose of 200 mg/kg. In another embodiment, an antisense oligomer (e.g., golodirsen), or pharmaceutically acceptable salt thereof, is administered at a dose of 300 mg/kg. In some embodiments, the human subject is 7 to 13 years of age (inclusive).

In some embodiments, the methods of the present disclosure comprise administering the pharmaceutical composition comprising an antisense oligomer (e.g., casimersen), or pharmaceutically acceptable salt thereof, to a male human subject, wherein the antisense oligomer, or pharmaceutically acceptable salt thereof, is administered at a dose of about 80 to about 300 mg/kg. In one embodiment, an antisense oligomer (e.g., casimersen), or pharmaceutically acceptable salt thereof, is administered at a dose of about 100 mg/kg. In another embodiment, an antisense oligomer (e.g., casimersen), or pharmaceutically acceptable salt thereof, is administered at a dose of 200 mg/kg. In another embodiment, an antisense oligomer (e.g., casimersen), or pharmaceutically acceptable salt thereof, is administered at a dose of 300 mg/kg. In some embodiments, the human subject is 7 to 13 years of age (inclusive).

In some embodiments, the pharmaceutical composition comprising an antisense oligomer (e.g., eteplirsen), or pharmaceutically acceptable salt thereof, further comprises a pharmaceutically acceptable carrier. In some embodiments, the pharmaceutically acceptable carrier is a saline solution that includes a phosphate buffer, e.g., a phosphate-buffered saline.

In some embodiments, the methods of the present disclosure increase the number of dystrophin-positive fibers in the human subject. In other embodiments, the human subjects treated with the methods of the disclosure achieve a higher North American Ambulatory Assessment (NSAA) total score. In some embodiments, the higher NSAA score is achieved at about week 24, about week 48, about week 60, about week 80, about week 100, about week 120, about week 140, about week 150, about week 160, about week 180, or about week 200, relative to baseline. In one embodiment, the higher NSAA score is achieved at about week 144, relative to baseline. In another embodiment, the higher NSAA score is achieved at about week 164, relative to baseline.

In some embodiment, the methods of the disclosure reduce loss of ambulation, relative to baseline in the human subject, as measured by the 6 Minute Walk Test (6MWT). In some embodiment, ambulation is maintained, relative to baseline. In other embodiments, ambulation is improved, relative to baseline.

In yet other embodiments, the methods of the disclosure reduces loss of pulmonary function in the human subject, relative to baseline. The loss of pulmonary function can be measured as % annual decline rate in Forced Vital Capacity (FVC).

In some embodiments, the methods of the disclosure include administering another therapeutic agent, such as a steroid, to the subject.

In certain embodiments, the methods of the disclosure comprise administering to a human subject with a DMD having a mutation of the DMD gene that is amenable to exon 44, exon 45, exon 50, exon 51, exon 52, or exon 53 skipping a pharmaceutical composition comprising an antisense oligomer, or pharmaceutically acceptable salt thereof, as an intravenous infusion at a dose of about 100 mg/kg once weekly for up to 24 weeks. In one embodiment, the human subject is male of 7 to 13 years of age (inclusive). In one embodiment of the method, the antisense oligomer is eteplirsen, golodirsen, or casimersen.

In certain other embodiments, the methods of the disclosure comprise administering to a human subject with a DMD having a mutation of the DMD gene that is amenable to exon 44, exon 45, exon 50, exon 51, exon 52, or exon 53 skipping a pharmaceutical composition comprising an antisense oligomer, or pharmaceutically acceptable salt thereof, as an intravenous infusion at a dose of about 100 mg/kg once weekly for up to 48 weeks. In one embodiment, the human subject is male of 7 to 13 years of age (inclusive). In one embodiment of the method, the antisense oligomer is eteplirsen, golodirsen, or casimersen.

In certain embodiments, the methods of the disclosure comprise administering to a human subject with a DMD having a mutation of the DMD gene that is amenable to exon 44, exon 45, exon 50, exon 51, exon 52, or exon 53 skipping a pharmaceutical composition comprising an antisense oligomer, or pharmaceutically acceptable salt thereof, as an intravenous infusion at a dose of about 100 mg/kg once weekly for up to 144 weeks. In one embodiment, the human subject is male of 7 to 13 years of age (inclusive). In one embodiment of the method, the antisense oligomer is eteplirsen, golodirsen, or casimersen.

In certain embodiments, the methods of the disclosure comprise administering to a human subject with a DMD having a mutation of the DMD gene that is amenable to exon 44, exon 45, exon 50, exon 51, exon 52, or exon 53 skipping a pharmaceutical composition comprising an antisense oligomer, or pharmaceutically acceptable salt thereof, administered as an intravenous infusion at a dose of about 200 mg/kg once weekly for up to 24 weeks. In one embodiment, the human subject is male of 7 to 13 years of age (inclusive). In one embodiment of the method, the antisense oligomer is eteplirsen, golodirsen, or casimersen.

In certain other embodiments, the methods of the disclosure comprise administering to a human subject with a DMD having a mutation of the DMD gene that is amenable to exon 44, exon 45, exon 50, exon 51, exon 52, or exon 53 skipping a pharmaceutical composition comprising an antisense oligonucleotide, administered as an intravenous infusion at a dose of about 200 mg/kg once weekly for up to 48 weeks. In one embodiment, the human subject is male of 7 to 13 years of age (inclusive). In one embodiment of the method, the antisense oligomer is eteplirsen, golodirsen, or casimersen.

In certain other embodiments, the methods of the disclosure comprise administering to a human subject with a DMD having a mutation of the DMD gene that is amenable to exon 44, exon 45, exon 50, exon 51, exon 52, or exon 53 skipping a pharmaceutical composition comprising an antisense oligonucleotide administered as an intravenous infusion at a dose of about 300 mg/kg once weekly for up to 48 weeks. In one embodiment, the human subject is male of 7 to 13 years of age (inclusive). In one embodiment of the method, the antisense oligomer is eteplirsen, golodirsen, or casimersen.

In certain embodiments, the methods of the disclosure comprise administering to a human subject with a DMD having a mutation of the DMD gene that is amenable to exon 44, exon 45, exon 50, exon 51, exon 52, or exon 53 skipping a pharmaceutical composition comprising an antisense oligomer, or pharmaceutically acceptable salt thereof, administered as an intravenous infusion at a dose of about 200 mg/kg once weekly for up to 144 weeks. In one embodiment, the human subject is male of 7 to 13 years of age (inclusive). In one embodiment of the method, the antisense oligomer is eteplirsen, golodirsen, or casimersen.

In certain embodiments, the methods of the disclosure comprise administering to a human subject with a DMD having a mutation of the DMD gene that is amenable to exon 44, exon 45, exon 50, exon 51, exon 52, or exon 53 skipping a pharmaceutical composition comprising an antisense oligomer, or pharmaceutically acceptable salt thereof, administered as an intravenous infusion at a dose of about 300 mg/kg once weekly for up to 144 weeks. In one embodiment, the human subject is male of 7 to 13 years of age (inclusive). In one embodiment of the method, the antisense oligomer is eteplirsen, golodirsen, or casimersen.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 depicts increase in % exon 51 skipping in hDMD Δ52 mdx mice following administration of high-dose eteplirsen.

FIG. 2 depicts dystrophin production in hDMD Δ52 mdx mice following administration of high-dose eteplirsen.

FIG. 3 depicts results of grip strength test in hDMD Δ52 mdx mice following administration of high-dose eteplirsen.

FIG. 4 depicts exon skipping in non-human primates (NHP) in quadriceps, heart, and diaphragm following administration of high-dose eteplirsen.

FIG. 5 compares exon skipping in NHPs in quadriceps following intravenous or subcutaneous administration of high-dose eteplirsen.

FIG. 6 provides images of stained myotube cultures treated with increasing concentrations of golodirsen (with myosin heavy chain in red and dystrophin in green).

FIG. 7 provides high content analysis results of the dystrophin staining intensity measures at different treatment concentration.

### DETAILED DESCRIPTION

Embodiments of the present invention relate to improved methods for treating muscular dystrophy, such as DMD and BMD, by administering antisense compounds that are specifically designed to induce exon skipping in the human dystrophin gene. Dystrophin plays a vital role in muscle function, and various muscle-related diseases are characterized by mutated forms of this gene. Hence, in certain embodiments, the improved methods described herein may be used for inducing exon skipping in mutated forms of the human dystrophin gene, such as the mutated dystrophin genes found in DMD and BMD.

Due to aberrant mRNA splicing events caused by mutations, these mutated human dystrophin genes either express defective dystrophin protein or express no measurable dystrophin at all, a condition that leads to various forms of muscular dystrophy. To remedy this condition, the antisense compounds of the present invention hybridize to selected regions of a pre-processed RNA of a mutated human dystrophin gene, induce exon skipping and differential splicing in that otherwise aberrantly spliced dystrophin mRNA, and thereby allow muscle cells to produce an mRNA transcript that encodes a functional dystrophin protein. In certain embodiments, the resulting dystrophin protein is not necessarily the “wild-type” form of dystrophin, but is rather an internally truncated, yet functional or semi-functional, form of dystrophin.

By increasing the levels of functional dystrophin protein in muscle cells, these and related embodiments are useful in the prophylaxis and treatment of muscular dystrophy, especially those forms of muscular dystrophy, such as DMD and BMD, that are characterized by the expression of defective dystrophin proteins due to aberrant mRNA splicing. The methods described herein further provide improved treatment options for patients with muscular dystrophy and offer significant and practical advantages over alternate methods of treating relevant forms of muscular dystrophy. For example, in some embodiments, the improved methods relate to the administration of an antisense compound for inducing exon skipping in the human dystrophin gene at a higher dose and/or for a longer duration than prior approaches.

Thus, the invention relates to improved methods for treating muscular dystrophy such as DMD and BMD, by inducing exon skipping in a patient. In some embodiments, exon skipping is induced by administering an effective amount of a composition which includes a charge-neutral, phosphorodiamidate morpholino oligomer (PMO), such as eteplirsen, which selectively binds to a target sequence in an exon of dystrophin pre-mRNA. In some embodiments, the invention relates to methods of treating DMD or BMD in which an effective amount of a composition e.g., about 80 mg/kg to about 300 mg/kg, which includes an antisense oligomer, or pharmaceutically acceptable salt thereof, as described herein, such as eteplirsen, over a period of time sufficient to treat the disease.

In certain embodiments, the antisense oligomer, or pharmaceutically acceptable salt thereof, is complementary to one or more exons or a portion thereof in the transcript. In certain embodiments, the one or more exons or a portion thereof are selected from group consisting of exon 44, exon 45, exon 50, exon 51, exon 52, or exon 53, and any combination thereof. In certain embodiments, the antisense oligomer, or pharmaceutically acceptable salt thereof, induces skipping of exon 51, exon 45, or exon 53 of the dystrophin transcript.

In some embodiments, the antisense oligomer, or pharmaceutically acceptable salt thereof, induces skipping of exon 53 of the dystrophin transcript. In some embodiments, the antisense oligomer, or pharmaceutically acceptable salt thereof, induces skipping of exon 45 of the dystrophin transcript. In some embodiments, the antisense oligomer, or pharmaceutically acceptable salt thereof, induces skipping of exon 44 of the dystrophin transcript. In some embodiments, the antisense oligomer, or pharmaceutically acceptable salt thereof, induces skipping of exon 50 of the dystrophin transcript. In some embodiments, the antisense oligomer, or pharmaceutically acceptable salt thereof, induces skipping of exon 51 of the dystrophin transcript. In some embodiments, the antisense oligomer, or pharmaceutically acceptable salt thereof, induces skipping of exon 52 of the dystrophin transcript.

In some embodiments, the methods of the disclosure comprise administering a pharmaceutical composition comprising an antisense oligomer, or pharmaceutically acceptable salt thereof, the antisense oligomer, or pharmaceutically acceptable salt thereof, is complementary to one or more exons or a portion thereof in the dystrophin transcript. In some embodiments, the one or more exons or a portion thereof are selected from the group consisting of exon 44, exon 45, exon 50, exon 51, exon 52, exon 53. In some embodiments, the antisense oligomer, or pharmaceutically acceptable salt thereof, induces skipping of exon 51, exon 45, or exon 53 of the dystrophin transcript. In some embodiments, the antisense oligomer, or pharmaceutically acceptable salt thereof, induces skipping of exon 51 of the dystrophin transcript. In some embodiments, the antisense oligomer, or pharmaceutically acceptable salt thereof, induces skipping of exon 45 of the dystrophin transcript. In some embodiments, the antisense oligomer, or pharmaceutically acceptable salt thereof, induces skipping of exon 53 of the dystrophin transcript.

Various mutations in the dystrophin gene are amenable to exon 51 skipping. Non-limiting examples of mutations in the following exons are amenable to exon 51 skipping include, e.g.: 45-50, 47-50, 48-50, 49-50, 50, 52, 52-63 (Leiden Duchenne muscular dystrophy mutation database, Leiden University Medical Center, The Netherlands). Determining whether a patient has a mutation in the DMD gene that is amenable to exon skipping is well within the purview of one of skill in the art (see, e.g., Aartsma-Rus et al. (2009) Hum Mut 30:293-299).

In certain embodiments, the antisense oligomer, or pharmaceutically acceptable salt thereof, induces skipping of exon 51 of the dystrophin transcript. In certain embodiments, the antisense oligomer, or pharmaceutically acceptable salt thereof, comprises a base sequence that is complementary to an exon 51 target region of the dystrophin transcript designated as an annealing site, wherein the base sequence and annealing site are selected from:

| Annealing Site | Base Sequence [5′ to 3′] | SEQ ID NO. |
|---|---|---|
| H51A(+66+95) | CTC CAA CAT CAA GGA AGA TGG CAT TTC | 1 |
| | TAG | |
| H51A(+74+97) | ACC TCC AAC ATC AAG GAA GAT GGC | 2 |
| H51A(+70+99) | GTA CCT CCA ACA TCA AGG AAG ATG GCA | 3 |
| | TTT | |
| H51A(+72+99) | GTA CCT CCA ACA TCA AGG AAG ATG GCA T | 4 |
| H51A(+68+87) | TCA AGG AAG ATG GCA TTT CT | 5 |
| H51A(+68+87) | UCA AGG AmAGm AmUGm GmCA UUU CU | 6 |

wherein T and U of each of SEQ ID NOS: 1-6 are each thymine or uracil. In certain embodiments, the T and U in the antisense oligomer are both thymine. In certain embodiments, the T and U in the antisense oligomer are both uracil. In certain embodiments, the annealing site is H51A(+66+95).

In certain embodiments, the antisense oligomer, or pharmaceutically acceptable salt thereof, induces skipping of exon 53 of the dystrophin transcript. In certain embodiments, the antisense oligomer, or pharmaceutically acceptable salt thereof, comprises a base sequence that is complementary to an exon 53 target region of the dystrophin transcript designated as an annealing site, wherein the base sequence and annealing site are selected from:

| Annealing Site | Targeting Sequence [5′ to 3′] | SEQ ID NO: |
|---|---|---|
| H53A(+36+60) | GTT GCC TCC GGT TCT GAA GGT GTT C | 7 |
| H53A(+36+60) | GTT G5mC5mC T5mC5mC GGT T5mC T GAA | 8 |
| | GGT GTT 5mC | |
| H53A(+36+56) | CCT CCG GTT CTG AAG GTG TTC | 9 |
| H53A(+23+47) | CTG AAG GTG TTC TTG TAC TTC ATC C | 10 |
| H53A(+32+56) | CCT CCG GTT CTG AAG GTG TTC TTG T | 11 |
| H53A(+33+60) | GTT GCC TCC GGT TCT GAA GGT GTT CTT | 12 |
| | G | |
| H53A(+30+59) | TTG CCT CCG GTT CTG AAG GTG TTC TTG | 13 |
| | TAC | |
| H53A(+39+62) | CTG TTG CCT CCG GTT CTG AAG GTG | 14 |
| H53A(+39+69) | CAT TCA ACT GTT GCC TCC GGT TCT GAA | 15 |
| | GGT G | |
| H53A(+45+62) | CTG TTG CCT CCG GTT CTG | 16 |

wherein T and U of each of SEQ ID NOS: 7-16 are each thymine or uracil. In certain embodiments, the T and U in the antisense oligomer are both thymine. In certain embodiments, the T and U in the antisense oligomer are both uracil. In certain embodiments, the annealing site is H53A(+36+60). In certain embodiments, the annealing site is H53A(+36+56).

In certain embodiments, the antisense oligomer, or pharmaceutically acceptable salt thereof, induces skipping of exon 45 of the dystrophin transcript. In certain embodiments, the antisense oligomer, or pharmaceutically acceptable salt thereof, comprises a base sequence that is complementary to an exon 45 target region of the dystrophin transcript designated as an annealing site, wherein the base sequence and annealing site are selected from:

| Annealing Site | Base Sequence [5′ to 3′] | SEQ ID NO: |
|---|---|---|
| H45A(−03+19) | CAA TGC CAT CCT GGA GTT CCT G | 17 |
| H45A(−09+25) | GCT GCC CAA TGC CAT CCT GGA GTT CCT GTA | 18 |
| | AGA T | |
| H45A(−03+25) | GCT GCC CAA TGC CAT CCT GGA GTT CCT G | 19 |
| H45A(−06+25) | GCT GCC CAA TGC CAT CCT GGA GTT CCT GTA A | 20 |
| H45A(−12+19) | CAA TGC CAT CCT GGA GTT CCT GTA AGA TAC | 21 |
| | C | |
| H45A(−09+19) | CAA TGC CAT CCT GGA GTT CCT GTA AGA T | 22 |
| H45A(−12+16) | TGC CAT CCT GGA GTT CCT GTA AGA TAC C | 23 |
| H45A(−14+25) | GCT GCC CAA TGC CAT CCT GGA GTT CCT GTA | 24 |
| | AGA TAC CAA | |
| H45A(−08+19) | CAA TGC CAT CCT GGA GTT CCT GTA AGA | 25 |
| HM45A(−07+25) | GCT GCC CAA TGC CAT CCT GGA GTT CCT GTA | 26 |
| | AG | |
| H45A(−12+22) | GCC CAA TGC CAT CCT GGA GTT CCT GTA AGA | 27 |
| | TAC C | |
| H45A(−09+22) | GCC CAA TGC CAT CCT GGA GTT CCT GTA AGA | 28 |
| | T | |
| H45A(−09+30) | TTG CCG CTG CCC AAT GCC ATC CTG GAG TTC | 29 |
| | CTG TAA GAT | |
| H45A(−06+22) | GCC CAA TGC CAT CCT GGA GTT CCT GTA A | 30 |
| H45A(−06+28) | GCC GCT GCC CAA TGC CAT CCT GGA GTT CCT | 31 |
| | GTA A | |
| H45A(−03+22) | GCC CAA TGC CAT CCT GGA GTT CCT G | 32 |
| H45A(−03+28) | GCC GCT GCC CAA TGC CAT CCT GGA GTT CCT G | 33 |
| H45A(+9+26) | m5C-G-m5C-T-G-C-m5C-m5C-A-A-T-G-m5C-m5C-A- | 34 |
| | U-m5C-m5C | |

wherein T and U of each of SEQ ID NOS: 18-34 are each thymine or uracil. In certain embodiments, the T and U in the antisense oligomer are both thymine. In certain embodiments, the T and U in the antisense oligomer are both uracil. In certain embodiments, the annealing site is H45A(−03+19).

In certain embodiments, the antisense oligomer, or pharmaceutically acceptable salt thereof, induces skipping of exon 44 of the dystrophin transcript. In certain embodiments, the antisense oligomer, or pharmaceutically acceptable salt thereof, comprises a base sequence that is complementary to an exon 44 target region of the dystrophin transcript designated as an annealing site, wherein the base sequence and annealing site are selected from:

| Annealing Site | Base Sequence [5′ to 3′] | SEQ ID NO: |
|---|---|---|
| H44A(−10+15) | GAT CTG TCA AAT CGC CTG CAG GTA A | 35 |
| H44A(−07+15) | GAT CTG TCA AAT CGC CTG CAG G | 36 |
| H44M(−07+17) | CAG ATC TGT CAA ATC GCC TGC AGG | 37 |
| H44A(−08+15) | GAT CTG TCA AAT CGC CTG CAG GT | 38 |
| H44A(−06+15) | GAT CTG TCA AAT CGC CTG CAG | 39 |
| H44A(−08+17) | CAG ATC TGT CAA ATC GCC TGC AGG T | 40 |
| H44A(−06+17) | CAG ATC TGT CAA ATC GCC TGC AG | 41 |

wherein T of each of SEQ ID NOS: 35-41 is thymine or uracil. In certain embodiments, the T in the antisense oligomer is thymine. In certain embodiments, the T in the antisense oligomer is uracil.

In certain embodiments, the antisense oligomer, or pharmaceutically acceptable salt thereof, induces skipping of exon 50 of the dystrophin transcript. In certain embodiments, the antisense oligomer, or pharmaceutically acceptable salt thereof, comprises a base sequence that is complementary to an exon 50 target region of the dystrophin transcript designated as an annealing site, wherein the base sequence and annealing site are selected from:

| Annealing Site | Targeting Sequence [5′ to 3′] | SEQ ID NO: |
|---|---|---|
| H50D(+04-18) | GGG ATC CAG TAT ACT TAC AGG C | 42 |
| H50D(+07-18) | GGG ATC CAG TAT ACT TAC AGG CTC C | 43 |
| H50D(+07-16) | GAT CCA GTA TAC TTA CAG GCT CC | 44 |
| H50D(+07-17) | GGA TCC AGT ATA CTT ACA GGC TCC | 45 |
| H50A(−19+07) | ACT TCC TCT TTA ACA GAA AAG CAT AC | 46 |
| H50D(+07-15) | ATC CAG TAT ACT TAC AGG CTC C | 47 |
| H50A(−02+23) | GAG CTC AGA TCT TCT AAC TTC CTC T | 48 |
| H50D(+06-18) | GGG ATC CAG TAT ACT TAC AGG CTC | 49 |
| H50D(+07-20) | ATG GGA TCC AGT ATA CTT ACA GGC TCC | 50 |

wherein T of each of SEQ ID NOS: 42-50 is thymine or uracil. In certain embodiments, the T in the antisense oligomer is thymine. In certain embodiments, the T in the antisense oligomer is uracil.

In certain embodiments, the antisense oligomer, or pharmaceutically acceptable salt thereof, induces skipping of exon 52 of the dystrophin transcript. In certain embodiments, the antisense oligomer, or pharmaceutically acceptable salt thereof, comprises a base sequence that is complementary to an exon 52 target region of the dystrophin transcript designated as an annealing site, wherein the base sequence and annealing site are selected from:

| Annealing Site | Targeting Sequence [5′ to 3′] | SEQ ID NO: |
|---|---|---|
| H52A(−01+24) | CTG TTC CAA ATC CTG CAT TGT TGC C | 51 |

wherein T of SEQ ID NO: 51 is thymine or uracil. In certain embodiments, the T in the antisense oligomer is thymine. In certain embodiments, the T in the antisense oligomer is uracil.

In some embodiments, the antisense oligomer is according to Formula (I):

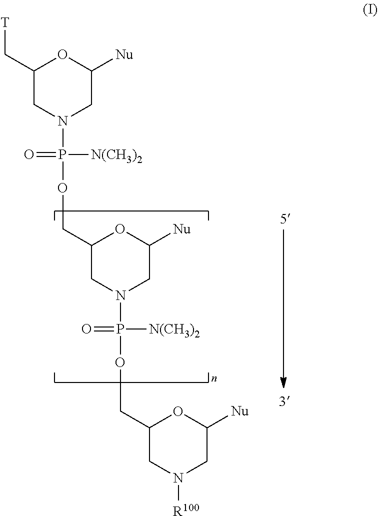

or a pharmaceutically acceptable salt thereof, wherein: each Nu is a nucleobase which taken together form a targeting sequence; T is defined below; each Nu from 1 to (n+1) and 5′ to 3′ corresponds to the nucleobases in one of the following:

| Annealing Site | Base Sequence [5′ to 3′] | SEQ ID NO. |
|---|---|---|
| H51A(+66+95) | CTC CAA CAT CAA GGA AGA TGG CAT TTC | 1 |
| | TAG | |
| H51A(+74+97) | ACC TCC AAC ATC AAG GAA GAT GGC | 2 |
| H51A(+70+99) | GTA CCT CCA ACA TCA AGG AAG ATG GCA | 3 |
| | TTT | |
| H51A(+72+99) | GTA CCT CCA ACA TCA AGG AAG ATG GCA T | 4 |
| H51A(+68+87) | TCA AGG AAG ATG GCA TTT CT | 5 |
| H51A(+68+87) | UCA AGG AAG AUG GCA UUU CU | 52 |
| H53A(+36+60) | GTT GCC TCC GGT TCT GAA GGT GTT C | 7 |
| H53A(+36+60) | GTT GCC TCC GGT TC T GAA GGT GTT C | 53 |
| H53A(+36+56) | CCT CCG GTT CTG AAG GTG TTC | 9 |
| H53A(+23+47) | CTG AAG GTG TTC TTG TAC TTC ATC C | 10 |
| H53A(+32+56) | CCT CCG GTT CTG AAG GTG TTC TTG T | 11 |
| H53A(+33+60) | GTT GCC TCC GGT TCT GAA GGT GTT CTT G | 12 |
| H53A(+30+59) | TTG CCT CCG GTT CTG AAG GTG TTC TTG | 13 |
| | TAC | |
| H53A(+39+62) | CTG TTG CCT CCG GTT CTG AAG GTG | 14 |
| H53A(+39+69) | CAT TCA ACT GTT GCC TCC GGT TCT GAA | 15 |
| | GGT G | |
| H53A(+45+62) | CTG TTG CCT CCG GTT CTG | 16 |
| H45A(−03+19) | CAA TGC CAT CCT GGA GTT CCT G | 17 |
| H45A(−09+25) | GCT GCC CAA TGC CAT CCT GGA GTT CCT | 18 |
| | GTA AGA T | |
| H45A(−03+25) | GCT GCC CAA TGC CAT CCT GGA GTT CCT G | 19 |
| H45A(−06+25) | GCT GCC CAA TGC CAT CCT GGA GTT CCT | 20 |
| | GTA A | |
| H45A(−12+19) | CAA TGC CAT CCT GGA GTT CCT GTA AGA | 21 |
| | TAC C | |
| H45A(−09+19) | CAA TGC CAT CCT GGA GTT CCT GTA AGA T | 22 |
| H45A(−12+16) | TGC CAT CCT GGA GTT CCT GTA AGA TAC C | 23 |
| H45A(−14+25) | GCT GCC CAA TGC CAT CCT GGA GTT CCT | 24 |
| | GTA AGA TAC CAA | |
| H45A(−08+19) | CAA TGC CAT CCT GGA GTT CCT GTA AGA | 25 |
| HM45A(−07+25) | GCT GCC CAA TGC CAT CCT GGA GTT CCT | 26 |
| | GTA AG | |
| H45A(−12+22) | GCC CAA TGC CAT CCT GGA GTT CCT GTA | 27 |
| | AGA TAC C | |
| H45A(−09+22) | GCC CAA TGC CAT CCT GGA GTT CCT GTA | 28 |
| | AGA T | |
| H45A(−09+30) | TTG CCG CTG CCC AAT GCC ATC CTG GAG | 29 |
| | TTC CTG TAA GAT | |
| H45A(−06+22) | GCC CAA TGC CAT CCT GGA GTT CCT GTA A | 30 |
| H45A(−06+28) | GCC GCT GCC CAA TGC CAT CCT GGA GTT | 31 |
| | CCT GTA A | |
| H45A(−03+22) | GCC CAA TGC CAT CCT GGA GTT CCT G | 32 |
| H45A(−03+28) | GCC GCT GCC CAA TGC CAT CCT GGA GTT | 33 |
| | CCT G | |
| H45A(+9+26) | CGCT GCC CAA TGC CAU CC | 54 |
| H44A(−10+15) | GAT CTG TCA AAT CGC CTG CAG GTA A | 35 |
| H44A(−07+15) | GAT CTG TCA AAT CGC CTG CAG G | 36 |
| H44M(−07+17) | CAG ATC TGT CAA ATC GCC TGC AGG | 37 |
| H44A(−08+15) | GAT CTG TCA AAT CGC CTG CAG GT | 38 |
| H44A(−06+15) | GAT CTG TCA AAT CGC CTG CAG | 39 |
| H44A(−08+17) | CAG ATC TGT CAA ATC GCC TGC AGG T | 40 |
| H44A(−06+17) | CAG ATC TGT CAA ATC GCC TGC AG | 41 |
| H50D(+04-18) | GGG ATC CAG TAT ACT TAC AGG C | 42 |
| H50D(+07-18) | GGG ATC CAG TAT ACT TAC AGG CTC C | 43 |
| H50D(+07-16) | GAT CCA GTA TAC TTA CAG GCT CC | 44 |
| H50D(+07-17) | GGA TCC AGT ATA CTT ACA GGC TCC | 45 |
| H50A(−19+07) | ACT TCC TCT TTA ACA GAA AAG CAT AC | 46 |
| H50D(+07-15) | ATC CAG TAT ACT TAC AGG CTC C | 47 |
| H50A(−02+23) | GAG CTC AGA TCT TCT AAC TTC CTC T | 48 |
| H50D(+06-18) | GGG ATC CAG TAT ACT TAC AGG CTC | 49 |
| H50D(+07-20) | ATG GGA TCC AGT ATA CTT ACA GGC TCC | 50 |
| H52A(−01+24) | CTG TTC CAA ATC CTG CAT TGT TGC C | 51 |

wherein each U and T in the antisense oligomer is independently thymine or uracil. In some embodiments, each T and U in the antisense oligomer is thymine.

In one embodiment of the methods, the annealing site is H51A(+66+95), H53A(+36+60), H53A(+36+56), or H45A(−03+19).

The T moiety attached to the 5′ end of the antisense oligomer of Formula (I) is selected from:

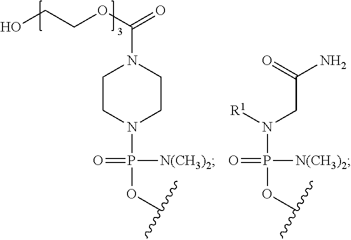

and

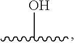

wherein R¹ is C₁-C₆ alkyl.

In one embodiment, the methods comprise administering eteplirsen [sequence 5′-CTCCAACATCAAGGAAGATGGCATTTCTAG-3′] (SEQ ID NO:1), a PMO designed to skip exon 51 of the human dystrophin gene in patients with DMD who are amenable to exon 51 skipping to restore the reading frame and produce a functional shorter form of the dystrophin protein. Eteplirsen is registered under CAS Registry Number 1173755-55-9. Chemical names include: RNA, [P-deoxy-P-(dimethylamino)](2′,3′-dideoxy-2′,3′-imino-2′,3′-seco)(2′a→5′)(C-m5U-C-C-A-A-C-A-m5U-C-A-A-G-G-A-A-G-A-m5U-G-G-C-A-m5U-m5U-m5U-C-m5U-A-G), 5′-[P-[4-[[2-[2-(2-hydroxyethoxy)ethoxy]ethoxy]carbonyl]-1-piperazinyl]-N,N-dimethylphosphonamidate] and P,2′,3′-trideoxy-P-(dimethylamino)-5′-O-{P-[4-(10-hydroxy-2,5,8-trioxadecanoyl)piperazin-1-yl]-N,N-dimethylphosphonamidoyl}-2′,3′-imino-2′,3′-secocytidylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoadenylyl-(2′a→5)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoguanylyl-(2′a→5)-P,2′,3′-trideoxy-P-(dimethylamino)-2′,3′-imino-2′,3′-secoadenylyl-(2′a→5)-P,2′,3′-trideoxy-P-(dimethylamino)-2′,3′-imino-2′,3′-secoadenylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoadenylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secoguanylyl-(2′a→5′)-P,2′,3′-trideoxy-P-(dimethylamino)-2′,3′-imino-2′,3′-secocytidylyl-(2′a→5′)-P,2′,3′-trideoxy-P-(dimethylamino)-2′,3′-imino-2′,3′-secoadenylyl-(2′a→5)-P,3′-dideoxy-P-(dimethylamino)-2′,3′-imino-2′,3′-secothymidylyl-(2′a→5′)-P,3′-dideoxy-P-(dimethylamino)-2′,3′-imino-2′,3′-secothymidylyl-(2′a→5′)-P,3′-dideoxy-P-(dimethylamino)-2′,3′-imino-2′,3′-secothymidylyl-(2′a→5′)-P,2′,3′-trideoxy-P-(dimethylamino)-2′,3′-imino-2′,3′-secocytidylyl-(2′a→5′)-P,3′-dideoxy-P-(dimethylamino)-2′,3′-imino-2′,3′-secothymidylyl-(2′a→5)-P,2′,3′-trideoxy-P-(dimethylamino)-2′,3′-imino-2′,3′-secoadenylyl-(2′a→5′)-2′,3′-dideoxy-2′,3′-imino-2′,3′-secoguanosine. Eteplirsen's structure is depicted as following:

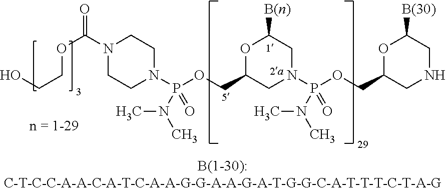

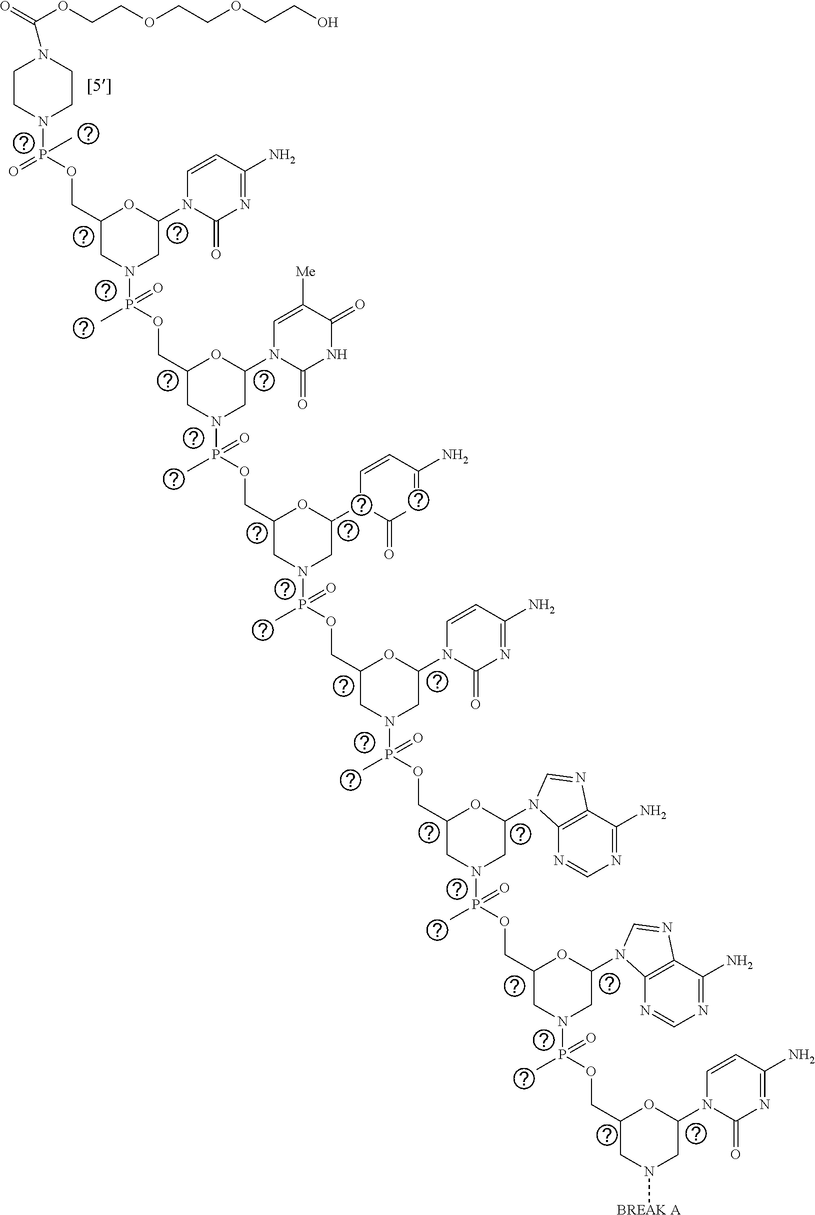

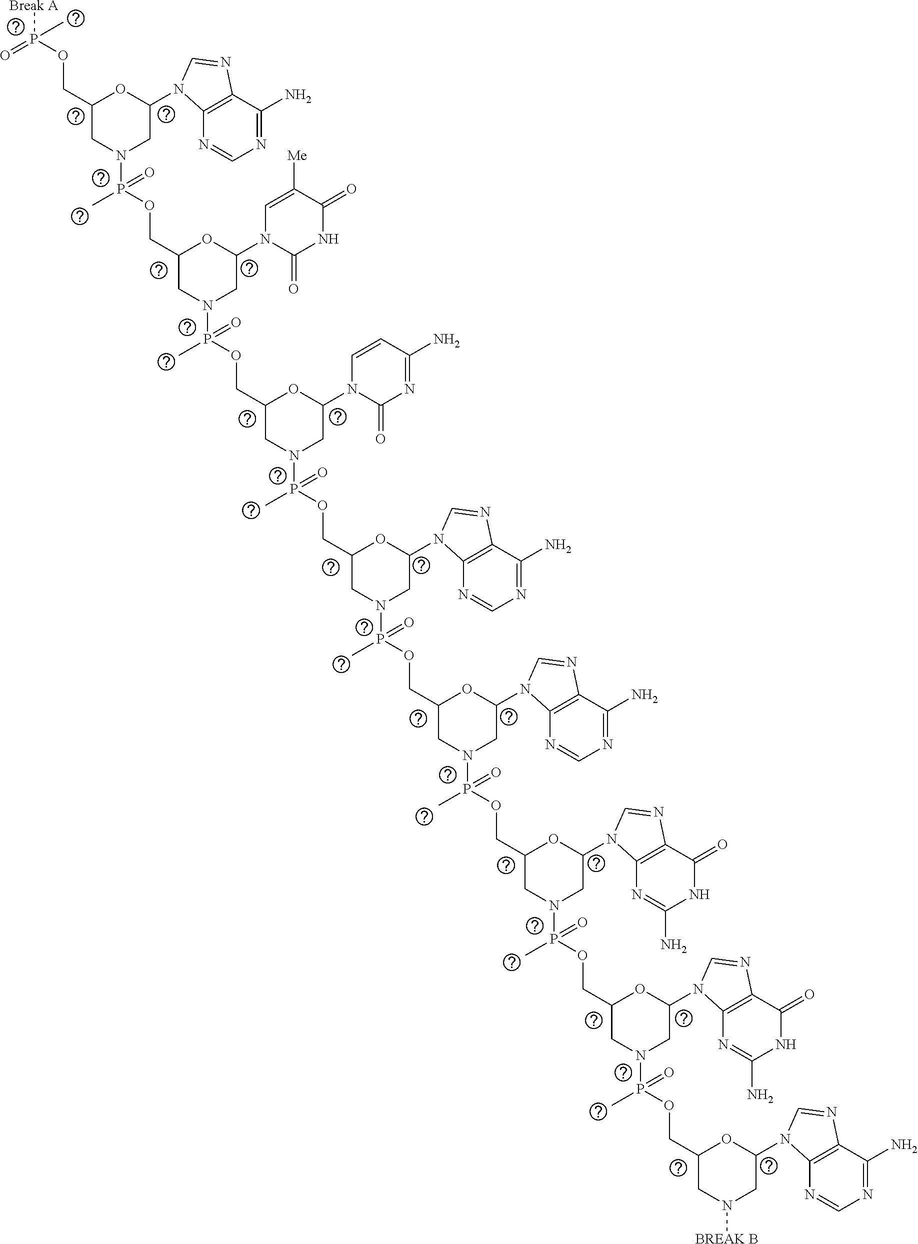

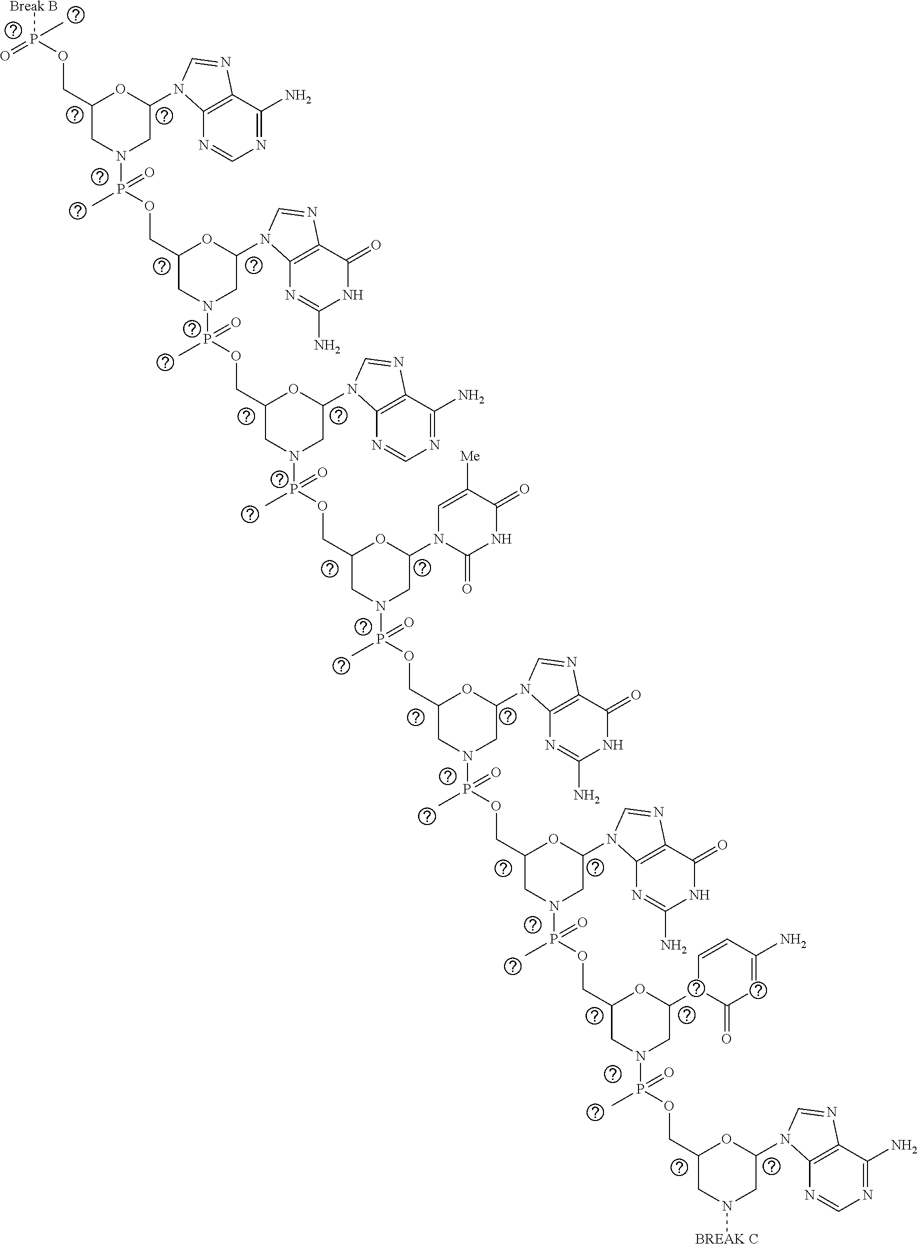

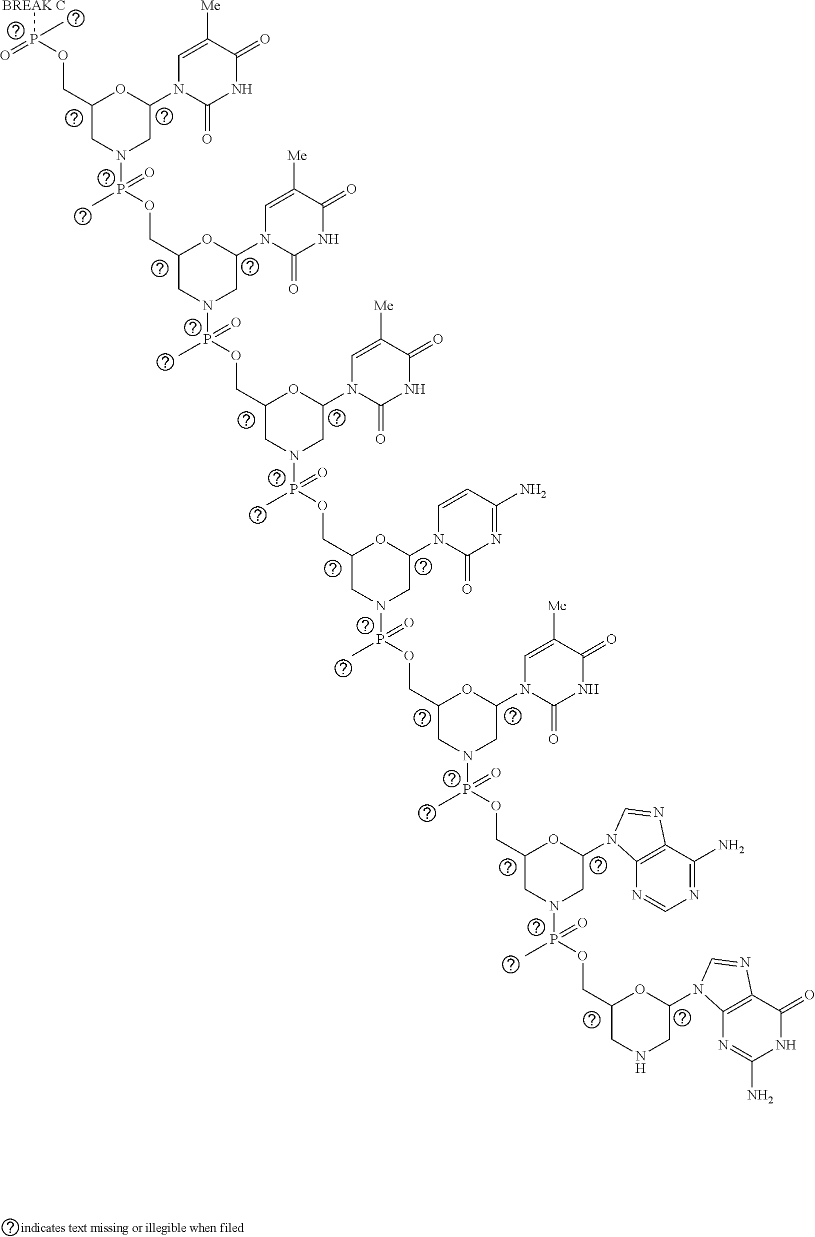

In one embodiment, the methods comprise administering golodirsen also known by its code name “SRP-4053.” Golodirsen is a PMO having the base sequence 5′-GTTGCCTCCGGTTCTGAAGGTGTTC-3′ (SEQ ID NO: 7). Golodirsen is registered under CAS Registry Number 1422959-91-8. Chemical names include: all-P-ambo-[P,2′,3′-trideoxy-P-(dimethylamino)-2′,3′-imino-2′,3′-seco](2′a→5)(G-T-T-G-C-C-T-C-C-G-G-T-T-C-T-G-A-A-G-G-T-G-T-T-C) 5′-[4-({2-[2-(2-hydroxyethoxy)ethoxy]ethoxy}carbonyl)-N,N-dimethylpiperazine-1-phosphonamidate]. Golodirsen's structure is depicted as following:

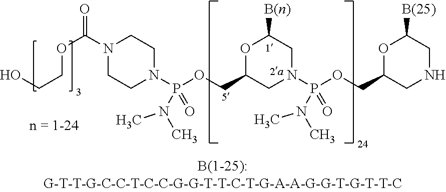

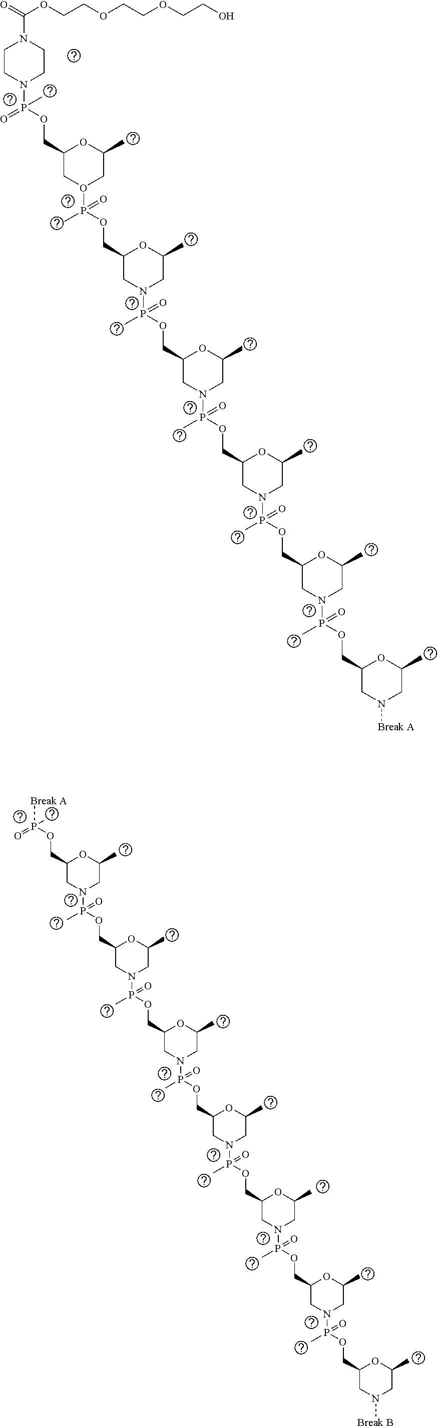

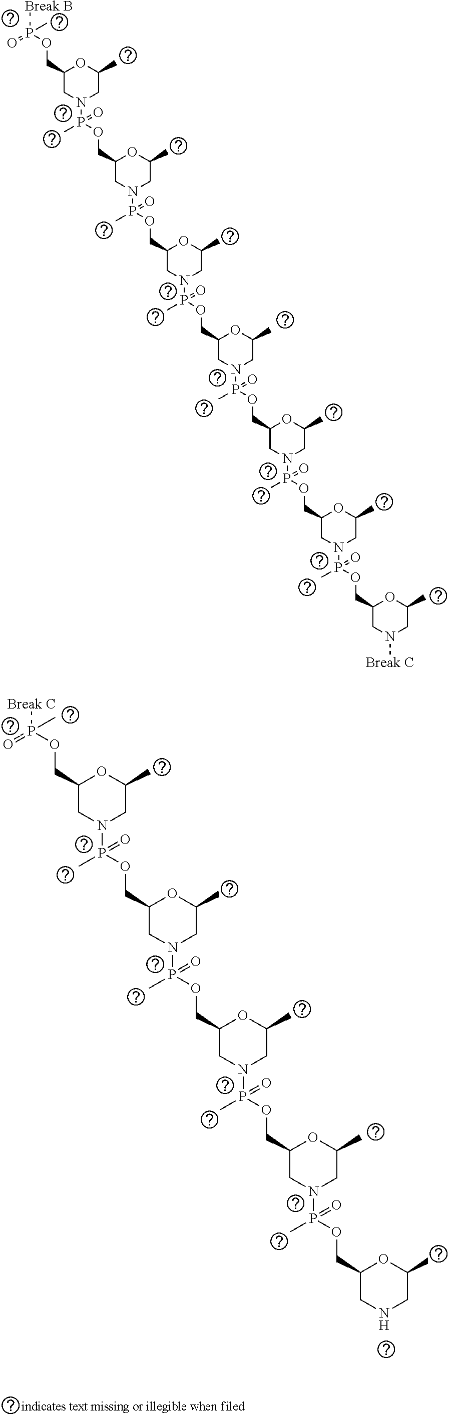

In one embodiment, the antisense oligomer is casimersen also known by its code name “SPR-4045” is a PMO having the base sequence 5′-CAATGCCATCCTGGAGTTCCTG-3′ (SEQ ID NO: 17). Casimersen is registered under CAS Registry Number 1422959-91-8. Chemical names include: all-P-ambo-[P,2′,3′-trideoxy-P-(dimethylamino)-2′,3′-imino-2′,3′-seco](2′a→5)(C-A-A-T-G-C-C-A-T-C-C-T-G-G-A-G-T-T-C-C-T-G) 5′-[4-({2-[2-(2-hydroxyethoxy)ethoxy]ethoxy}carbonyl)-N,N-dimethylpiperazine-1-phosphonamidate].

Casimersen's structure is depicted as following:

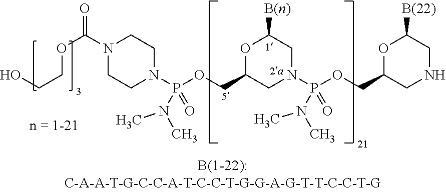

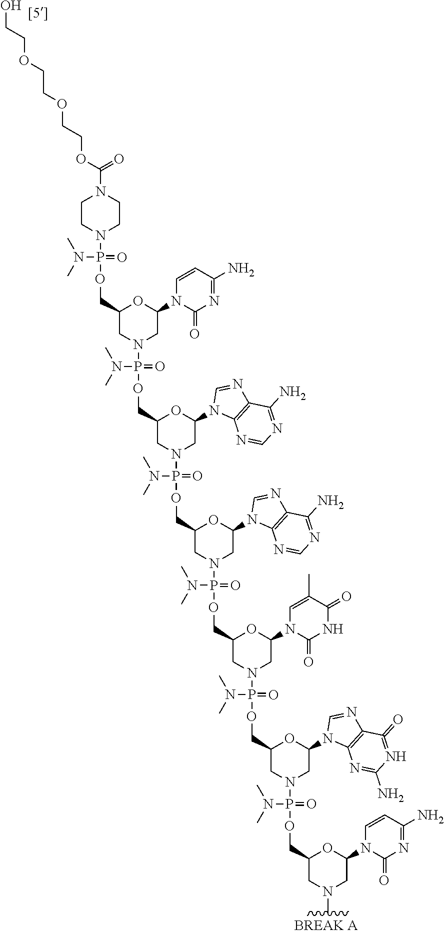

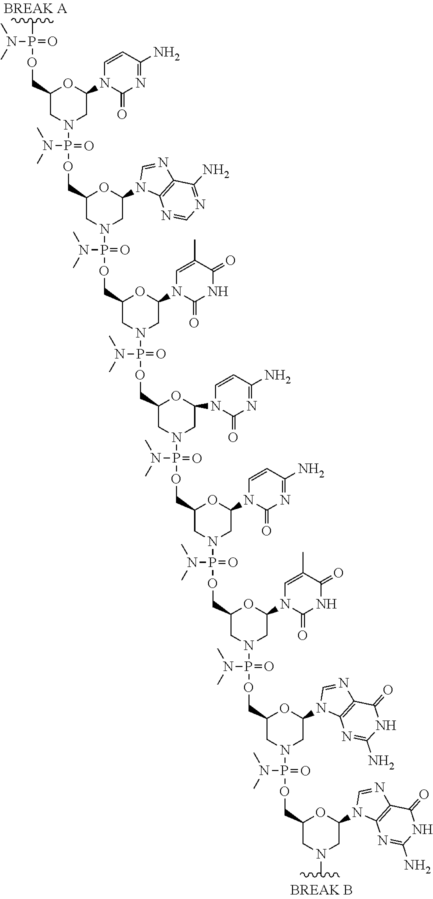 [SMILES: *N1C[C@@H](COP(=O)(N(C)C)N2C[C@@H](COP(=O)(N(C)C)N3C[C@@H](COP(=O)(N(C)C)N4C[C@@H](COP(=O)(N(C)C)N5C[C@@H](COP(=O)(N(C)C)N6C[C@@H](COP(=O)(N(C)C)N7C[C@@H](COP(=O)(N(C)C)N8C[C@@H](COP(*)(=O)N(C)C)O[C@@H](n9ccc(N)nc9=O)C8)O[C@@H](n8cnc9c(N)ncnc98)C7)O[C@@H](n7cc(C)c(=O)[nH]c7=O)C6)O[C@@H](n6ccc(N)nc6=O)C5)O[C@@H](n5ccc(N)nc5=O)C4)O[C@@H](n4cc(C)c(=O)[nH]c4=O)C3)O[C@@H](n3cnc4c(=O)[nH]c(N)nc43)C2)O[C@@H](n2cnc3c(=O)[nH]c(N)nc32)C1]

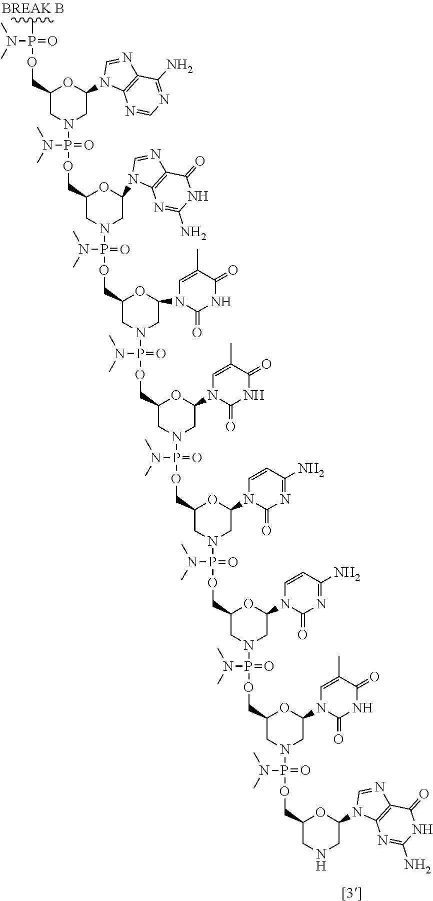 [SMILES: *P(=O)(OC[C@@H]1CN(P(=O)(OC[C@@H]2CN(P(=O)(OC[C@@H]3CN(P(=O)(OC[C@@H]4CN(P(=O)(OC[C@@H]5CN(P(=O)(OC[C@@H]6CN(P(=O)(OC[C@@H]7CN(P(=O)(OC[C@@H]8CNC[C@H](n9cnc%10c(=O)[nH]c(N)nc%109)O8)N(C)C)C[C@H](n8cc(C)c(=O)[nH]c8=O)O7)N(C)C)C[C@H](n7ccc(N)nc7=O)O6)N(C)C)C[C@H](n6ccc(N)nc6=O)O5)N(C)C)C[C@H](n5cc(C)c(=O)[nH]c5=O)O4)N(C)C)C[C@H](n4cc(C)c(=O)[nH]c4=O)O3)N(C)C)C[C@H](n3cnc4c(=O)[nH]c(N)nc43)O2)N(C)C)C[C@H](n2cnc3c(N)ncnc32)O1)N(C)C]

For clarity, structures of the disclosure including, for example, the above structures of eteplirsen, golodirsen, and casimersen, are continuous from 5′ to 3′, and, for the convenience of depicting the entire structure in a compact form, various illustration breaks labeled “BREAK A” and “BREAK B” have been included. As would be understood by the skilled artisan, for example, each indication of “BREAK A” shows a continuation of the illustration of the structure at these points. The skilled artisan understands that the same is true for each instance of “BREAK B” in the structures above. None of the illustration breaks, however, are intended to indicate, nor would the skilled artisan understand them to mean, an actual discontinuation of the structure above.

Embodiments of the disclosure relate to methods for treating muscular dystrophy, such as DMD and BMD, by administering an antisense oligomer, or pharmaceutically acceptable salt thereof, either per se or as a pharmaceutical composition. Dystrophin plays a vital role in muscle function, and various muscle-related diseases are characterized by mutated forms of this gene. Hence, in certain embodiments, the methods described herein may be used for inducing exon skipping in mutated forms of the human dystrophin gene, such as the mutated dystrophin genes found in DMD and BMD.

Due to aberrant mRNA splicing events caused by mutations, these mutated human dystrophin genes either express defective dystrophin protein or express no measurable dystrophin at all, a condition that leads to various forms of muscular dystrophy. To remedy this condition, an antisense oligomer hybridizes to exon 44, exon 45, exon 50, exon 51, exon 52, or exon 53 of a pre-processed RNA of a mutated human dystrophin gene, induces exon skipping and differential splicing in that otherwise aberrantly spliced dystrophin mRNA, and thereby allows muscle cells to produce an mRNA transcript that encodes a functional dystrophin protein. In certain embodiments, the resulting dystrophin protein is not necessarily the “wild-type” form of dystrophin, but is rather a truncated, yet functional or semi-functional, form of dystrophin.

By increasing the levels of functional dystrophin protein in muscle cells, these and related embodiments are useful in the prophylaxis and treatment of muscular dystrophy, especially those forms of muscular dystrophy, such as DMD and BMD, that are characterized by the expression of defective dystrophin proteins due to aberrant mRNA splicing. The methods described herein further provide treatment options for patients with muscular dystrophy and offer significant and practical advantages over alternate methods of treating relevant forms of muscular dystrophy. For example, in some embodiments, the methods relate to the administration of an antisense oligomer, or pharmaceutically acceptable salt thereof, for inducing exon skipping in the human dystrophin gene at a higher dose and/or for a longer duration than prior approaches.

Thus, the disclosure relates to methods for treating muscular dystrophy such as DMD and BMD, by inducing exon 44, exon 45, exon 50, exon 51, exon 52, or exon 53 skipping in a human subject. In some embodiments, exon skipping is induced by administering a pharmaceutical composition comprising an antisense oligomer (e.g., eteplirsen), or pharmaceutically acceptable salt thereof, wherein the antisense oligomer, or pharmaceutically acceptable salt thereof, is administered at a dose of about 80 to about 300 mg/kg. In some embodiments, the disclosure relates to methods of treating DMD or BMD, comprising administering to a human subject a pharmaceutical composition comprising an antisense oligomer (e.g., eteplirsen), or pharmaceutically acceptable salt thereof, wherein the antisense oligomer, or pharmaceutically acceptable salt thereof, is administered at a dose of about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg. In some embodiments, an antisense oligomer (e.g., eteplirsen), or pharmaceutically acceptable salt thereof, is administered at a dose of about 100 mg/kg. In other embodiments, an antisense oligomer (e.g., eteplirsen), or pharmaceutically acceptable salt thereof, is administered at a dose of about 200 mg/kg. In other embodiments, an antisense oligomer (e.g., eteplirsen), or pharmaceutically acceptable salt thereof, is administered at a dose of about 300 mg/kg.

In some embodiments, exon skipping is induced by administering a pharmaceutical composition comprising an antisense oligomer (e.g., golodirsen), or pharmaceutically acceptable salt thereof, wherein the antisense oligomer, or pharmaceutically acceptable salt thereof, is administered at a dose of about 80 to about 300 mg/kg. In some embodiments, the disclosure relates to methods of treating DMD or BMD, comprising administering to a human subject a pharmaceutical composition comprising an antisense oligomer (e.g., golodirsen), or pharmaceutically acceptable salt thereof, wherein the antisense oligomer, or pharmaceutically acceptable salt thereof, is administered at a dose of about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg. In some embodiments, an antisense oligomer (e.g., golodirsen), or pharmaceutically acceptable salt thereof, is administered at a dose of about 100 mg/kg. In other embodiments, an antisense oligomer (e.g., eteplirsen), or pharmaceutically acceptable salt thereof, is administered at a dose of about 200 mg/kg. In other embodiments, an antisense oligomer (e.g., golodirsen), or pharmaceutically acceptable salt thereof, is administered at a dose of about 300 mg/kg.

In some embodiments, exon skipping is induced by administering a pharmaceutical composition comprising an antisense oligomer (e.g., casimersen), or pharmaceutically acceptable salt thereof, wherein the antisense oligomer, or pharmaceutically acceptable salt thereof, is administered at a dose of about 80 to about 300 mg/kg. In some embodiments, the disclosure relates to methods of treating DMD or BMD, comprising administering to a human subject a pharmaceutical composition comprising an antisense oligomer (e.g., casimersen), or pharmaceutically acceptable salt thereof, wherein the antisense oligomer, or pharmaceutically acceptable salt thereof, is administered at a dose of about 100 mg/kg, about 125 mg/kg, about 150 mg/kg, about 175 mg/kg, about 200 mg/kg, about 225 mg/kg, about 250 mg/kg, about 275 mg/kg, or about 300 mg/kg. In some embodiments, an antisense oligomer (e.g., casimersen), or pharmaceutically acceptable salt thereof, is administered at a dose of about 100 mg/kg. In other embodiments, an antisense oligomer (e.g., eteplirsen), or pharmaceutically acceptable salt thereof, is administered at a dose of about 200 mg/kg. In other embodiments, an antisense oligomer (e.g., casimersen), or pharmaceutically acceptable salt thereof, is administered at a dose of about 300 mg/kg.

In some embodiments, the methods of the disclosure comprise administering a pharmaceutical composition comprising an antisense oligomer (e.g., eteplirsen), or pharmaceutically acceptable salt thereof, once weekly. In some embodiments, the composition comprising an antisense oligomer (e.g., eteplirsen), or pharmaceutically acceptable salt thereof, is administered twice weekly. In yet other embodiments, the composition comprising an antisense oligomer (e.g., eteplirsen), or pharmaceutically acceptable salt thereof, is administered once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, or once every 12 weeks. In some embodiments, the pharmaceutical composition comprising an antisense oligomer (e.g., eteplirsen), or pharmaceutically acceptable salt thereof, is administered once monthly.

In some embodiments, the methods of the disclosure comprise administering a pharmaceutical composition comprising an antisense oligomer (e.g., golodirsen), or pharmaceutically acceptable salt thereof, once weekly. In some embodiments, the composition comprising an antisense oligomer (e.g., golodirsen), or pharmaceutically acceptable salt thereof, is administered twice weekly. In yet other embodiments, the composition comprising an antisense oligomer (e.g., golodirsen), or pharmaceutically acceptable salt thereof, is administered once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, or once every 12 weeks. In some embodiments, the pharmaceutical composition comprising an antisense oligomer (e.g., golodirsen), or pharmaceutically acceptable salt thereof, is administered once monthly.

In some embodiments, the methods of the disclosure comprise administering a pharmaceutical composition comprising an antisense oligomer (e.g., casimersen), or pharmaceutically acceptable salt thereof, once weekly. In some embodiments, the composition comprising an antisense oligomer (e.g., casimersen), or pharmaceutically acceptable salt thereof, is administered twice weekly. In yet other embodiments, the composition comprising an antisense oligomer (e.g., casimersen), or pharmaceutically acceptable salt thereof, is administered once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, or once every 12 weeks. In some embodiments, the pharmaceutical composition comprising an antisense oligomer (e.g., casimersen), or pharmaceutically acceptable salt thereof, is administered once monthly.

In some embodiments, the methods comprise administering a pharmaceutical composition comprising an antisense oligomer (e.g., eteplirsen), or pharmaceutically acceptable salt thereof, for up to about 24 weeks, up to about 48 weeks, up to about 60 weeks, up to about 80 weeks, up to about 100 weeks, up to about 120 weeks, up to about 140 weeks, up to about 150 weeks, up to about 160 weeks, up to about 180 weeks, up to about 200 weeks. In some embodiments, the pharmaceutical composition comprising an antisense oligomer (e.g., eteplirsen), or pharmaceutically acceptable salt thereof, is administered for up to about 144 weeks. In some embodiments, the composition comprising an antisense oligomer (e.g., eteplirsen), or pharmaceutically acceptable salt thereof, is administered for at least 24 weeks, at least 36 weeks, at least 48 weeks, at least 120 weeks, at least 144 weeks, or at least 164 weeks. In yet other embodiments, the pharmaceutical composition comprising an antisense oligomer (e.g., eteplirsen), or pharmaceutically acceptable salt thereof, is administered for the duration of the illness.

In some embodiments, the methods comprise administering a pharmaceutical composition comprising an antisense oligomer (e.g., golodirsen), or pharmaceutically acceptable salt thereof, for up to about 24 weeks, up to about 48 weeks, up to about 60 weeks, up to about 80 weeks, up to about 100 weeks, up to about 120 weeks, up to about 140 weeks, up to about 150 weeks, up to about 160 weeks, up to about 180 weeks, up to about 200 weeks. In some embodiments, the pharmaceutical composition comprising an antisense oligomer (e.g., golodirsen), or pharmaceutically acceptable salt thereof, is administered for up to about 144 weeks. In some embodiments, the composition comprising an antisense oligomer (e.g., golodirsen), or pharmaceutically acceptable salt thereof, is administered for at least 24 weeks, at least 36 weeks, at least 48 weeks, at least 120 weeks, at least 144 weeks, or at least 164 weeks. In yet other embodiments, the pharmaceutical composition comprising an antisense oligomer (e.g., golodirsen), or pharmaceutically acceptable salt thereof, is administered for the duration of the illness.

In some embodiments, the methods comprise administering a pharmaceutical composition comprising an antisense oligomer (e.g., casimersen), or pharmaceutically acceptable salt thereof, for up to about 24 weeks, up to about 48 weeks, up to about 60 weeks, up to about 80 weeks, up to about 100 weeks, up to about 120 weeks, up to about 140 weeks, up to about 150 weeks, up to about 160 weeks, up to about 180 weeks, up to about 200 weeks. In some embodiments, the pharmaceutical composition comprising an antisense oligomer (e.g., casimersen), or pharmaceutically acceptable salt thereof, is administered for up to about 144 weeks. In some embodiments, the composition comprising an antisense oligomer (e.g., casimersen), or pharmaceutically acceptable salt thereof, is administered for at least 24 weeks, at least 36 weeks, at least 48 weeks, at least 120 weeks, at least 144 weeks, or at least 164 weeks. In yet other embodiments, the pharmaceutical composition comprising an antisense oligomer (e.g., casimersen), or pharmaceutically acceptable salt thereof, is administered for the duration of the illness.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the disclosure, preferred methods and materials are described. For the purposes of the disclosure, the following terms are defined below.

### I. Definitions

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The terms “antisense oligomer” and “antisense compound” and “antisense oligonucleotide” are used interchangeably and refer to a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid: oligomer heteroduplex within the target sequence.

The terms “morpholino oligomer” or “PMO” (phosphoramidate- or phosphorodiamidate morpholino oligomer) refer to an oligonucleotide analog composed of morpholino subunit structures, where (i) the structures are linked together by phosphorus-containing linkages, one to three atoms long, preferably two atoms long, and preferably uncharged or cationic, joining the morpholino nitrogen of one subunit to a 5′ exocyclic carbon of an adjacent subunit, and (ii) each morpholino ring bears a purine or pyrimidine base-pairing moiety effective to bind, by base specific hydrogen bonding, to a base in a polynucleotide. The synthesis, structures, and binding characteristics of morpholino oligomers are detailed in U.S. Pat. Nos. 5,698,685, 5,217,866, 5,142,047, 5,034,506, 5,166,315, 5,521,063, 5,506,337, 8,076,476, 8,299,206 and 7,943,762 (cationic linkages), all of which are incorporated herein by reference.

An “exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA. The human dystrophin gene has about 79 exons.

An “intron” refers to a nucleic acid region (within a gene) that is not translated into a protein. An intron is a non-coding section that is transcribed into a precursor mRNA (pre-mRNA), and subsequently removed by splicing during formation of the mature RNA.

“Exon skipping” refers generally to the process by which an entire exon, or a portion thereof, is removed from a given pre-processed RNA, and is thereby excluded from being present in the mature RNA, such as the mature mRNA that is translated into a protein. Hence, the portion of the protein that is otherwise encoded by the skipped exon is not present in the expressed form of the protein, typically creating an altered, though still functional, form of the protein.

“Dystrophin” is a rod-shaped cytoplasmic protein, and a vital part of the protein complex that connects the cytoskeleton of a muscle fiber to the surrounding extracellular matrix through the cell membrane. Dystrophin contains multiple functional domains. For instance, dystrophin contains an actin binding domain at about amino acids 14-240 and a central rod domain at about amino acids 253-3040. This large central domain is formed by 24 spectrin-like triple-helical elements of about 109 amino acids, which have homology to alpha-actinin and spectrin. The repeats are typically interrupted by four proline-rich non-repeat segments, also referred to as hinge regions. Repeats 15 and 16 are separated by an 18 amino acid stretch that appears to provide a major site for proteolytic cleavage of dystrophin. The sequence identity between most repeats ranges from 10-25%. One repeat contains three alpha-helices: 1, 2 and 3. Alpha-helices 1 and 3 are each formed by 7 helix turns, probably interacting as a coiled-coil through a hydrophobic interface. Alpha-helix 2 has a more complex structure and is formed by segments of four and three helix turns, separated by a Glycine or Proline residue. Each repeat is encoded by two exons, typically interrupted by an intron between amino acids 47 and 48 in the first part of alpha-helix 2. The other intron is found at different positions in the repeat, usually scattered over helix-3. Dystrophin also contains a cysteine-rich domain at about amino acids 3080-3360), including a cysteine-rich segment (i.e., 15 Cysteines in 280 amino acids) showing homology to the C-terminal domain of the slime mold (Dictyostelium discoideum) alpha-actinin. The carboxy-terminal domain is at about amino acids 3361-3685.

The amino-terminus of dystrophin binds to F-actin and the carboxy-terminus binds to the dystrophin-associated protein complex (DAPC) at the sarcolemma. The DAPC includes the dystroglycans, sarcoglycans, integrins and caveolin, and mutations in any of these components cause autosomally inherited muscular dystrophies. The DAPC is destabilized when dystrophin is absent, which results in diminished levels of the member proteins, and in turn leads to progressive fibre damage and membrane leakage. In various forms of muscular dystrophy, such as Duchenne's muscular dystrophy (DMD) and Becker's muscular dystrophy (BMD), muscle cells produce an altered and functionally defective form of dystrophin, or no dystrophin at all, mainly due to mutations in the gene sequence that lead to incorrect splicing. The predominant expression of the defective dystrophin protein, or the complete lack of dystrophin or a dystrophin-like protein, leads to rapid progression of muscle degeneration, as noted above. In this regard, a “defective” dystrophin protein may be characterized by the forms of dystrophin that are produced in certain subjects with DMD or BMD, as known in the art, or by the absence of detectable dystrophin.

“Amenable to exon 53 skipping” as used herein with regard to a subject or patient is intended to include subjects and patients having one or more mutations in the dystrophin gene which, absent the skipping of exon 53 of the dystrophin gene, causes the reading frame to be out-of-frame thereby disrupting translation of the pre-mRNA leading to an inability of the subject or patient to produce dystrophin. Non-limiting examples of mutations in the following exons of the dystrophin gene are amenable to exon 53 skipping include, e.g., deletion of: exons 3 to 52, 4 to 52, 5 to 52, 6 to 52, 9 to 52, 10 to 52, 11 to 52, 13 to 52, 14 to 52, 15 to 52, 16 to 52, 17 to 52, 19 to 52, 21 to 52, 23 to 52, 24 to 52, 25 to 52, 26 to 52, 27 to 52, 28 to 52, 29 to 52, 30 to 52, 31 to 52, 32 to 52, 33 to 52, 34 to 52, 35 to 52, 36 to 52, 37 to 52, 38 to 52, 39 to 52, 40 to 52, 41 to 52, 43 to 52, 42 to 52, 45 to 52, 47 to 52, 48 to 52, 49 to 52, 50 to 52, 54 to 58, 54 to 61, 54 to 63, 54 to 64, 54 to 66, 54 to 76, 54 to 77, or exon 52. Determining whether a patient has a mutation in the dystrophin gene that is amenable to exon skipping is well within the purview of one of skill in the art (see, e.g., Aartsma-Rus et al. (2009) Hum Mutat. 30:293-299, Gurvich et al., Hum Mutat. 2009; 30(4) 633-640, and Fletcher et al. (2010) Molecular Therapy 18(6) 1218-1223.).

“Amenable to exon 45 skipping” as used herein with regard to a subject or patient is intended to include subjects and patients having one or more mutations in the dystrophin gene which, absent the skipping of exon 45 of the dystrophin pre-mRNA, causes the reading frame to be out-of-frame thereby disrupting translation of the pre-mRNA leading to an inability of the subject or patient to produce functional or semi-functional dystrophin. Examples of mutations in the dystrophin gene that are amenable to exon 45 skipping include, e.g., mutations in exons 7-44, 12-44, 18-44, 44, 46, 46-47, 46-48, 46-49, 46-51, 46-53, 46-55, 46-57, 46-59, 46-60, 46-67, 46-69, 46-75, and 46-78 (Leiden Duchenne muscular dystrophy mutation database, Leiden University Medical Center, The Netherlands). Determining whether a patient has a mutation in the dystrophin gene that is amenable to exon skipping is well within the purview of one of skill in the art (see, e.g., Aartsma-Rus et al. (2009) Hum Mutat. 30:293-299; Gurvich et al., Hum Mutat. 2009; 30(4) 633-640; and Fletcher et al. (2010) Molecular Therapy 18(6) 1218-1223).

As used herein, the terms “function” and “functional” and the like refer to a biological, enzymatic, or therapeutic function.

A “functional” dystrophin protein refers generally to a dystrophin protein having sufficient biological activity to reduce the progressive degradation of muscle tissue that is otherwise characteristic of muscular dystrophy, typically as compared to the altered or “defective” form of dystrophin protein that is present in certain subjects with DMD or BMD. In certain embodiments, a functional dystrophin protein may have about 10%, 20%, 30%, 40%, 50%, 60%, 70%, 80%, 90%, or 100% (including all integers in between) of the in vitro or in vivo biological activity of wild-type dystrophin, as measured according to routine techniques in the art. As one example, dystrophin-related activity in muscle cultures in vitro can be measured according to myotube size, myofibril organization (or disorganization), contractile activity, and spontaneous clustering of acetylcholine receptors (see, e.g., Brown et al., Journal of Cell Science. 112:209-216, 1999). Animal models are also valuable resources for studying the pathogenesis of disease, and provide a means to test dystrophin-related activity. Two of the most widely used animal models for DMD research are the mdx mouse and the golden retriever muscular dystrophy (GRMD) dog, both of which are dystrophin negative (see, e.g., Collins & Morgan, Int J Exp Pathol 84: 165-172, 2003). These and other animal models can be used to measure the functional activity of various dystrophin proteins. Included are truncated forms of dystrophin, such as those forms that are produced by certain of the exon-skipping antisense compounds of the disclosure.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a non-toxic, inert solid, semi-solid or liquid filler, diluent, encapsulating material, or formulation auxiliary of any type. Some examples of materials which can serve as pharmaceutically acceptable carriers are: sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; phosphate buffer solutions; non-toxic compatible lubricants such as sodium lauryl sulfate and magnesium stearate; coloring agents; releasing agents; coating agents; sweetening agents; flavoring agents; perfuming agents; preservatives; and antioxidants; according to the judgment of the formulator.

The term “restoration” of dystrophin synthesis or production refers generally to the production of a dystrophin protein including truncated forms of dystrophin in a patient with muscular dystrophy following treatment with an antisense oligonucleotide as described herein. In some embodiments, treatment results in an increase in novel dystrophin production in a patient. In some embodiments, treatment increases the number of dystrophin-positive fibers of normal in the subject. The percent of dystrophin-positive fibers in a patient following treatment can be determined by a muscle biopsy using known techniques. For example, a muscle biopsy may be taken from a suitable muscle, such as the biceps brachii muscle in a patient.

Analysis of the percentage of positive dystrophin fibers may be performed pre-treatment and/or post-treatment or at time points throughout the course of treatment. In some embodiments, a post-treatment biopsy is taken from the contralateral muscle from the pre-treatment biopsy. Pre- and post-treatment dystrophin expression studies may be performed using any suitable assay for dystrophin. In one embodiment, immunohistochemical detection is performed on tissue sections from the muscle biopsy using an antibody that is a marker for dystrophin, such as a monoclonal or a polyclonal antibody. For example, the MANDYS106 antibody can be used which is a highly sensitive marker for dystrophin. Any suitable secondary antibody may be used.

In some embodiments, the percent dystrophin-positive fibers are calculated by dividing the number of positive fibers by the total fibers counted. Normal muscle samples have 100% dystrophin-positive fibers. Therefore, the percent dystrophin-positive fibers can be expressed as a percentage of normal. To control for the presence of trace levels of dystrophin in the pretreatment muscle as well as revertant fibers a baseline can be set using sections of pre-treatment muscles from each patient when counting dystrophin-positive fibers in post-treatment muscles. This may be used as a threshold for counting dystrophin-positive fibers in sections of post-treatment muscle in that patient. In other embodiments, antibody-stained tissue sections can also be used for dystrophin quantification using Bioquant image analysis software (Bioquant Image Analysis Corporation, Nashville, Tenn.). The total dystrophin fluorescence signal intensity can be reported as a percentage of normal. In addition, Western blot analysis with monoclonal or polyclonal anti-dystrophin antibodies can be used to determine the percentage of dystrophin positive fibers. For example, the anti-dystrophin antibody NCL-Dys1 from Novacastra may be used. The percentage of dystrophin-positive fibers can also be analyzed by determining the expression of the components of the sarcoglycan complex (β,γ) and/or neuronal NOS.

“Treatment” of an individual (e.g. a mammal, such as a human subject) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Treatment includes any desirable effect on the symptoms or pathology of a disease or condition associated with the dystrophin protein, as in certain forms of muscular dystrophy, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “alkyl,” as used herein, unless otherwise specified, refers to a saturated straight or branched hydrocarbon. In certain embodiments, the alkyl group is a primary, secondary, or tertiary hydrocarbon. In certain embodiments, the alkyl group includes one to ten carbon atoms, i.e., C₁ to C₁₀ alkyl. In certain embodiments, the alkyl group includes one to six carbon atoms, i.e., C₁ to C₆ alkyl. The term includes both substituted and unsubstituted alkyl groups, including halogenated alkyl groups. In certain embodiments, the alkyl group is a fluorinated alkyl group. Non-limiting examples of moieties with which the alkyl group can be substituted are selected from the group consisting of halogen (fluoro, chloro, bromo, or iodo), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. In certain embodiments, the alkyl group is selected from the group consisting of methyl, CF₃, CCl₃, CFCl₂, CF₂Cl, ethyl, CH₂CF₃, CF₂CF₃, propyl, isopropyl, butyl, isobutyl, sec-butyl, t-butyl, pentyl, isopentyl, neopentyl, hexyl, isohexyl, 3-methylpentyl, 2,2-dimethylbutyl, and 2,3-dimethylbutyl.

The term “aryl” used alone or as part of a larger moiety as in “aralkyl,” “aralkoxy,” or “aryloxy-alkyl,” refers to aromatic ring groups having six to fourteen ring atoms, such as phenyl, 1-naphthyl, 2-naphthyl, 1-anthracyl and 2-anthracyl. An “aryl” ring may contain one or more substituents. The term “aryl” may be used interchangeably with the term “aryl ring.” “Aryl” also includes fused polycyclic aromatic ring systems in which an aromatic ring is fused to one or more rings. Non-limiting examples of useful aryl ring groups include phenyl, hydroxyphenyl, halophenyl, alkoxyphenyl, dialkoxyphenyl, trialkoxyphenyl, alkylenedioxyphenyl, naphthyl, phenanthryl, anthryl, phenanthro and the like, as well as 1-naphthyl, 2-naphthyl, 1-anthracyl and 2-anthracyl. Also included within the scope of the term “aryl,” as it is used herein, is a group in which an aromatic ring is fused to one or more non-aromatic rings, such as in an indanyl, phenanthridinyl, or tetrahydronaphthyl, where the radical or point of attachment is on the aromatic ring.

The term “acyl” refers to a C(O)R group (in which R signifies H, alkyl or aryl as defined herein). Examples of acyl groups include formyl, acetyl, benzoyl, phenylacetyl and similar groups.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′→3′),” is complementary to the nucleobase sequence “A-C-T (3′→5′).” Complementarity may be “partial,” in which less than all of the nucleobases of a given nucleobase sequence are matched to the other nucleobase sequence according to base pairing rules. For example, in some embodiments, complementarity between a given nucleobase sequence and the other nucleobase sequence may be about 70%, about 75%, about 80%, about 85%, about 90% or about 95%. Or, there may be “complete” or “perfect” (100%) complementarity between a given nucleobase sequence and the other nucleobase sequence to continue the example. The degree of complementarity between nucleobase sequences has significant effects on the efficiency and strength of hybridization between the sequences.

In one embodiment, treatment with the methods of the disclosure increases novel dystrophin production and slows or reduces the loss of ambulation that would be expected without treatment. For example, treatment may stabilize, maintain, improve or increase walking ability (e.g., stabilization of ambulation) in the subject. In some embodiments, treatment maintains or increases a stable walking distance in a patient, as measured by, for example, the 6 Minute Walk Test (6MWT), described by McDonald, et al. (Muscle Nerve, 2010; 42:966-74, herein incorporated by reference). A change in the 6 Minute Walk Distance (6MWD) may be expressed as an absolute value, a percentage change or a change in the %-predicted value. The performance of a DMD patient in the 6MWT relative to the typical performance of a healthy peer can be determined by calculating a %-predicted value. For example, the %-predicted 6MWD may be calculated using the following equation for males: 196.72+(39.81×age)−(1.36×age²)+(132.28×height in meters). For females, the %-predicted 6MWD may be calculated using the following equation: 188.61+(51.50×age)−(1.86×age²)+(86.10×height in meters) (Henricson et al. PLoS Curr., 2012, version 2, herein incorporated by reference). In some embodiments, treatment with an antisense oligonucleotide increases the stable walking distance in the patient from baseline.

Loss of muscle function in patients with DMD may occur against the background of normal childhood growth and development. Indeed, younger children with DMD may show an increase in distance walked during 6MWT over the course of about 1 year despite progressive muscular impairment. In some embodiments, the 6MWD from patients with DMD is compared to typically developing control subjects and to existing normative data from age and sex matched subjects. In some embodiments, normal growth and development can be accounted for using an age and height based equation fitted to normative data. Such an equation can be used to convert 6MWD to a percent-predicted (%-predicted) value in subjects with DMD. In certain embodiments, analysis of %-predicted 6MWD data represents a method to account for normal growth and development, and may show that gains in function at early ages (e.g., less than or equal to age 7) represent stable rather than improving abilities in patients with DMD (Henricson et al. PLoS Curr., 2012, version 2, herein incorporated by reference).

In some embodiments, treatment with an antisense oligomer (e.g., eteplirsen), or pharmaceutically acceptable salt thereof, of the disclosure slows or reduces the progressive respiratory muscle dysfunction and/or failure in patients with DMD that would be expected without treatment. In some embodiments, treatment with an antisense oligomer (e.g., eteplirsen), or pharmaceutically acceptable salt thereof, of the disclosure may reduce or eliminate the need for ventilation assistance that would be expected without treatment. In some embodiments, measurements of respiratory function for tracking the course of the disease, as well as the evaluation of potential therapeutic interventions include maximum inspiratory pressure (MIP), maximum expiratory pressure (MEP), and forced vital capacity (FVC). MIP and MEP measure the level of pressure a person can generate during inhalation and exhalation, respectively, and are sensitive measures of respiratory muscle strength. MIP is a measure of diaphragm muscle weakness.

In some embodiments, MEP may decline before changes in other pulmonary function tests, including MIP and FVC. In certain embodiments, MEP may be an early indicator of respiratory dysfunction. In certain embodiments, FVC may be used to measure the total volume of air expelled during forced exhalation after maximum inspiration. In patients with DMD, FVC increases concomitantly with physical growth until the early teens. However, as growth slows or is stunted by disease progression, and muscle weakness progresses, the vital capacity enters a descending phase and declines at an average rate of about 8 to 8.5 percent per year after 10 to 12 years of age. In certain embodiments, MIP percent predicted (MIP adjusted for weight), MEP percent predicted (MEP adjusted for age), and FVC percent predicted (FVC adjusted for age and height) are supportive analyses.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

### II. Methods of Treatment

The disclosure relates to improved methods for treating muscular dystrophy such as DMD and BMD, by inducing exon 44, exon 45, exon 50, exon 51, exon 52, or exon 53 skipping in a human subject. In these methods, exon skipping is induced by administering an antisense oligomer, or pharmaceutically acceptable salt thereof, either per se or as a pharmaceutical composition, wherein the antisense oligomer, or pharmaceutically acceptable salt thereof, is administered at a dose of about 80 to about 300 mg/kg.

In some embodiments, methods of the disclosure comprise administering a pharmaceutical composition comprising an antisense oligomer (e.g., eteplirsen), or pharmaceutically acceptable salt thereof, wherein the antisense oligomer, or pharmaceutically acceptable salt thereof, is administered in doses from about 80 mg to about 300 mg per kilogram of body weight per day or about 100 mg to about 200 mg per kilogram of body weight per day. In some cases, doses of greater than 300 mg/kg may be necessary. In some embodiments, doses for administration are from about 80 mg to about 300 mg/kg. In some embodiments, an antisense oligomer (e.g., eteplirsen), or pharmaceutically acceptable salt thereof, is administered at doses of about 80 mg/kg, about 85 mg/kg, about 90 mg/kg, about 95 mg/kg, about 100 mg/kg, about 105 mg/kg, about 110 mg/kg, about 120 mg/kg, about 125 mg/kg, about 130 mg/kg, about 135 mg/kg, about 140 mg/kg, about 145 mg/kg, about 150 mg/kg, about 155 mg/kg, about 160 mg/kg, about 165 mg/kg, about 170 mg/kg, about 175 mg/kg, about 180 mg/kg, about 185 mg/kg, about 190 mg/kg, about 195 mg/kg, about 200 mg/kg, about 205 mg/kg, about 210 mg/kg, about 215 mg/kg, about 220 mg/kg, about 225 mg/kg, about 230 mg/kg, about 235 mg/kg, about 240 mg/kg, about 245 mg/kg, about 250 mg/kg, about 255 mg/kg, about 260 mg/kg, about 265 mg/kg, about 270 mg/kg, about 275 mg/kg, about 280 mg/kg, about 285 mg/kg, about 290 mg/kg, about 295 mg/kg, or about 300 mg/kg. In one embodiment, an antisense oligomer (e.g., eteplirsen), or pharmaceutically acceptable salt thereof, is administered at a dose of about 100 mg/kg. In another embodiment, an antisense oligomer (e.g., eteplirsen), or pharmaceutically acceptable salt thereof, is administered at a dose of about 200 mg/kg. In another embodiment, an antisense oligomer (e.g., eteplirsen), or pharmaceutically acceptable salt thereof, is administered at a dose of about 300 mg/kg. In some embodiments, the administration is via intravenous (i.v.) infusion.

In some embodiments, methods of the disclosure comprise administering a pharmaceutical composition comprising an antisense oligomer (e.g., golodirsen), or pharmaceutically acceptable salt thereof, wherein the antisense oligomer, or pharmaceutically acceptable salt thereof, is administered in doses from about 80 mg to about 300 mg per kilogram of body weight per day or about 100 mg to about 200 mg per kilogram of body weight per day. In some cases, doses of greater than 300 mg/kg may be necessary. In some embodiments, doses for administration are from about 80 mg to about 300 mg/kg. In some embodiments, an antisense oligomer (e.g., golodirsen), or pharmaceutically acceptable salt thereof, is administered at doses of about 80 mg/kg, about 85 mg/kg, about 90 mg/kg, about 95 mg/kg, about 100 mg/kg, about 105 mg/kg, about 110 mg/kg, about 120 mg/kg, about 125 mg/kg, about 130 mg/kg, about 135 mg/kg, about 140 mg/kg, about 145 mg/kg, about 150 mg/kg, about 155 mg/kg, about 160 mg/kg, about 165 mg/kg, about 170 mg/kg, about 175 mg/kg, about 180 mg/kg, about 185 mg/kg, about 190 mg/kg, about 195 mg/kg, about 200 mg/kg, about 205 mg/kg, about 210 mg/kg, about 215 mg/kg, about 220 mg/kg, about 225 mg/kg, about 230 mg/kg, about 235 mg/kg, about 240 mg/kg, about 245 mg/kg, about 250 mg/kg, about 255 mg/kg, about 260 mg/kg, about 265 mg/kg, about 270 mg/kg, about 275 mg/kg, about 280 mg/kg, about 285 mg/kg, about 290 mg/kg, about 295 mg/kg, or about 300 mg/kg. In one embodiment, an antisense oligomer (e.g., golodirsen), or pharmaceutically acceptable salt thereof, is administered at a dose of about 100 mg/kg. In another embodiment, an antisense oligomer (e.g., golodirsen), or pharmaceutically acceptable salt thereof, is administered at a dose of about 200 mg/kg. In another embodiment, an antisense oligomer (e.g., golodirsen), or pharmaceutically acceptable salt thereof, is administered at a dose of about 300 mg/kg. In some embodiments, the administration is via intravenous (i.v.) infusion.

In some embodiments, methods of the disclosure comprise administering a pharmaceutical composition comprising an antisense oligomer (e.g., casimersen), or pharmaceutically acceptable salt thereof, wherein the antisense oligomer, or pharmaceutically acceptable salt thereof, is administered in doses from about 80 mg to about 300 mg per kilogram of body weight per day or about 100 mg to about 200 mg per kilogram of body weight per day. In some cases, doses of greater than 300 mg/kg may be necessary. In some embodiments, doses for administration are from about 80 mg to about 300 mg/kg. In some embodiments, an antisense oligomer (e.g., casimersen), or pharmaceutically acceptable salt thereof, is administered at doses of about 80 mg/kg, about 85 mg/kg, about 90 mg/kg, about 95 mg/kg, about 100 mg/kg, about 105 mg/kg, about 110 mg/kg, about 120 mg/kg, about 125 mg/kg, about 130 mg/kg, about 135 mg/kg, about 140 mg/kg, about 145 mg/kg, about 150 mg/kg, about 155 mg/kg, about 160 mg/kg, about 165 mg/kg, about 170 mg/kg, about 175 mg/kg, about 180 mg/kg, about 185 mg/kg, about 190 mg/kg, about 195 mg/kg, about 200 mg/kg, about 205 mg/kg, about 210 mg/kg, about 215 mg/kg, about 220 mg/kg, about 225 mg/kg, about 230 mg/kg, about 235 mg/kg, about 240 mg/kg, about 245 mg/kg, about 250 mg/kg, about 255 mg/kg, about 260 mg/kg, about 265 mg/kg, about 270 mg/kg, about 275 mg/kg, about 280 mg/kg, about 285 mg/kg, about 290 mg/kg, about 295 mg/kg, or about 300 mg/kg. In one embodiment, an antisense oligomer (e.g., casimersen), or pharmaceutically acceptable salt thereof, is administered at a dose of about 100 mg/kg. In another embodiment, an antisense oligomer (e.g., casimersen), or pharmaceutically acceptable salt thereof, is administered at a dose of about 200 mg/kg. In another embodiment, an antisense oligomer (e.g., casimersen), or pharmaceutically acceptable salt thereof, is administered at a dose of about 300 mg/kg. In some embodiments, the administration is via intravenous (i.v.) infusion.

If desired, the effective daily dose of an antisense oligomer (e.g., eteplirsen), or pharmaceutically acceptable salt thereof, may be administered as two, three, four, five, six or more sub-doses administered separately at appropriate intervals throughout the day, week, month, or year, either per se or as a pharmaceutical composition. In some embodiments, an antisense oligomer (e.g., eteplirsen), or pharmaceutically acceptable salt thereof, may be administered once every one, two, three, four or five years. In certain situations, dosing is one administration per day. In certain embodiments, dosing is one or more administration per every 2, 3, 4, 5, 6, 7, 8, 9, 10, 11, 12, 13, 14 days, or every 1, 2, 3, 4, 5, 6, 7, 8, 9, 10, 11, 12 weeks, or every 1, 2, 3, 4, 5, 6, 7, 8, 9, 10, 11, 12 months, as needed, to maintain the desired expression of a functional dystrophin protein. In some embodiments, dosing is one administration once every week. In some embodiments, dosing is one administration once every two weeks. In various embodiments, dosing is one or more administrations every month. In yet other embodiments, the antisense oligomer (e.g., eteplirsen), or pharmaceutically acceptable salt thereof, is administered once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, or once every 12 weeks. In some embodiments, the pharmaceutical composition comprising an antisense oligomer (e.g., eteplirsen), or pharmaceutically acceptable salt thereof, is administered once monthly. For other embodiments, an antisense oligomer (e.g., eteplirsen), or pharmaceutically acceptable salt thereof, may be administered once every one, two, three, four, five, six, seven, eight, nine, ten, eleven or twelve months.

In some embodiments, the pharmaceutical composition comprising an antisense oligomer (e.g., eteplirsen), or pharmaceutically acceptable salt thereof, is administered at regular intervals, e.g., daily; once every two days; once every three days; once every 3 to 7 days; once every 3 to 10 days; once every 7 to 10 days; once every week; once every two weeks; once monthly. For example, an antisense oligomer (e.g., eteplirsen), or pharmaceutically acceptable salt thereof, may be administered once weekly by intravenous infusion. For another example, an antisense oligomer (e.g., eteplirsen), or pharmaceutically acceptable salt thereof, may be administered once monthly by intravenous infusion. An antisense oligomer (e.g., eteplirsen), or pharmaceutically acceptable salt thereof, may be administered intermittently over a longer period of time, e.g., for several weeks, months or years. The treatment regimen may be adjusted (dose, frequency, route, etc.) as indicated, based on the results of immunoassays, other biochemical tests and physiological examination of the subject under treatment.

In various embodiments, an antisense oligomer (e.g., eteplirsen), or pharmaceutically acceptable salt thereof, either per se or as a pharmaceutical composition, is administered weekly at a dose of about 100 mg/kg. In various embodiments, an antisense oligomer (e.g., eteplirsen), or pharmaceutically acceptable salt thereof, is administered weekly at a dose of about 125 mg/kg. In various embodiments, an antisense oligomer (e.g., eteplirsen), or pharmaceutically acceptable salt thereof, is administered weekly at a dose of about 150 mg/kg. In various embodiments, an antisense oligomer (e.g., eteplirsen), or pharmaceutically acceptable salt thereof, is administered weekly at a dose of about 175 mg/kg. In various embodiments, an antisense oligomer (e.g., eteplirsen), or pharmaceutically acceptable salt thereof, is administered weekly at a dose of about 200 mg/kg. In various other embodiments, an antisense oligomer (e.g., eteplirsen), or pharmaceutically acceptable salt thereof, is administered weekly at a dose of about 300 mg/kg. In some embodiments, an antisense oligomer (e.g., eteplirsen), or pharmaceutically acceptable salt thereof, either per se or a pharmaceutical composition, is administered weekly at a dose of about 100 mg/kg via an intravenous infusion. In some embodiments, an antisense oligomer (e.g., eteplirsen), or pharmaceutically acceptable salt thereof, either per se or a pharmaceutical composition, is administered weekly at a dose of about 200 mg/kg via an intravenous infusion. As used herein, weekly is understood to have the art-accepted meaning of every week.

In various embodiments, an antisense oligomer (e.g., eteplirsen), or pharmaceutically acceptable salt thereof, either per se or as a pharmaceutical composition, is administered bi-weekly at a dose of about 100 mg/kg. In various embodiments, an antisense oligomer (e.g., eteplirsen), or pharmaceutically acceptable salt thereof, is administered bi-weekly at a dose of about 125 mg/kg. In various embodiments, an antisense oligomer (e.g., eteplirsen), or pharmaceutically acceptable salt thereof, is administered bi-weekly at a dose of about 150 mg/kg. In various embodiments, an antisense oligomer (e.g., eteplirsen), or pharmaceutically acceptable salt thereof, is administered bi-weekly at a dose of about 175 mg/kg. In various embodiments, an antisense oligomer (e.g., eteplirsen), or pharmaceutically acceptable salt thereof, is administered bi-weekly at a dose of about 200 mg/kg. In some embodiments, an antisense oligomer (e.g., eteplirsen), or pharmaceutically acceptable salt thereof, either per se or a pharmaceutical composition, is administered bi-weekly at a dose of about 100 mg/kg via an intravenous infusion. In some embodiments, an antisense oligomer (e.g., eteplirsen), or pharmaceutically acceptable salt thereof, either per se or a pharmaceutical composition, is administered bi-weekly at a dose of about 200 mg/kg via an intravenous infusion. As used herein, biweekly is understood to have the art-accepted meaning of every two weeks.

In various embodiments, an antisense oligomer (e.g., eteplirsen), or pharmaceutically acceptable salt thereof, either per se or as a pharmaceutical composition, is administered every third week at a dose of about 100 mg/kg. In various embodiments, an antisense oligomer (e.g., eteplirsen), or pharmaceutically acceptable salt thereof, is administered every third week at a dose of about 125 mg/kg. In various embodiments, an antisense oligomer (e.g., eteplirsen), or pharmaceutically acceptable salt thereof, is administered every third week at a dose of about 150 mg/kg. In various embodiments, an antisense oligomer (e.g., eteplirsen), or pharmaceutically acceptable salt thereof, is administered every third week at a dose of about 175 mg/kg. In various embodiments, an antisense oligomer (e.g., eteplirsen), or pharmaceutically acceptable salt thereof, is administered every third week at a dose of about 200 mg/kg. In some embodiments, an antisense oligomer (e.g., eteplirsen), or pharmaceutically acceptable salt thereof, either per se or a pharmaceutical composition, is administered every third week at a dose of about 100 mg/kg via an intravenous infusion. In some embodiments, an antisense oligomer (e.g., eteplirsen), or pharmaceutically acceptable salt thereof, either per se or a pharmaceutical composition, is administered every third week at a dose of about 200 mg/kg via an intravenous infusion. As used herein, every third week is understood to have the art-accepted meaning of once every three weeks.

In various embodiments, an antisense oligomer (e.g., eteplirsen), or pharmaceutically acceptable salt thereof, either per se or as a pharmaceutical composition, is administered monthly at a dose of about 100 mg/kg. In various embodiments, an antisense oligomer (e.g., eteplirsen), or pharmaceutically acceptable salt thereof, is administered monthly at a dose of about 125 mg/kg. In various embodiments, an antisense oligomer (e.g., eteplirsen), or pharmaceutically acceptable salt thereof, is administered monthly at a dose of about 150 mg/kg. In various embodiments, an antisense oligomer (e.g., eteplirsen), or pharmaceutically acceptable salt thereof, is administered monthly at a dose of about 175 mg/kg. In various embodiments, an antisense oligomer (e.g., eteplirsen), or pharmaceutically acceptable salt thereof, is administered monthly at a dose of about 200 mg/kg. In some embodiments, an antisense oligomer (e.g., eteplirsen), or pharmaceutically acceptable salt thereof, either per se or a pharmaceutical composition, is administered monthly at a dose of about 100 mg/kg via an intravenous infusion. In some embodiments, an antisense oligomer (e.g., eteplirsen), or pharmaceutically acceptable salt thereof, either per se or a pharmaceutical composition, is administered monthly at a dose of about 200 mg/kg via an intravenous infusion. As used herein, monthly is understood to have the art-accepted meaning of every month.

As would be understood in the art, weekly, biweekly, every third week, or monthly administrations may be in one or more administrations or sub-doses as discussed herein.

In certain embodiments, the time of intravenous infusion is from about 15 minutes to about 4 hours. In some embodiments, the time of infusion is from about 30 minutes to about 3 hours. In some embodiments, the time of infusion is from about 30 minutes to about 2 hours. In some embodiments, the time of infusion is from about 1 hour to about 2 hours. In some embodiments the time of infusion is from about 30 minutes to about 1 hour. In some embodiments, the time of infusion is about 60 minutes. In some embodiments, the time of infusion is 35 to 60 minutes.

In some embodiments, the methods of the disclosure comprise administering a pharmaceutical composition comprising an antisense oligomer (e.g., eteplirsen), or pharmaceutically acceptable salt thereof, wherein the composition is administered for up to about 24 weeks, up to about 25 weeks, up to about 26 weeks, up to about 27 weeks, up to about 28 weeks, up to about 29 weeks, up to about 30 weeks, up to about 31 weeks, up to about 32 weeks, up to about 33 weeks, up to about 34 weeks, up to about 35 weeks, up to about 36 weeks, up to about 37 weeks, up to about 38 weeks, up to about 39 weeks, up to about 40 weeks, up to about 41 weeks, up to about 42 weeks, up to about 43 weeks, up to about 44 weeks, up to about 45 weeks, up to about 46 weeks, up to about 47 weeks, up to about 48 weeks, up to about 49 weeks, up to about 50 weeks, up to about 51 weeks, up to about 52 weeks, up to about 53 weeks, up to about 54 weeks, up to about 55 weeks, up to about 56 weeks, up to about 57 weeks, up to about 58 weeks, up to about 59 weeks, up to about 60 weeks, up to about 61 weeks, up to about 62 weeks, up to about 63 weeks, up to about 64 weeks, up to about 65 weeks, up to about 66 weeks, up to about 67 weeks, up to about 68 weeks, up to about 69 weeks, up to about 70 weeks, up to about 71 weeks, about 72 weeks, up to about 73 weeks, up to about 74 weeks, up to about 75 weeks, up to about 76 weeks, up to about 77 weeks, up to about 78 weeks, up to about 79 weeks, up to about 80 weeks, up to about 81 weeks, up to about 82 weeks, up to about 83 weeks, up to about 84 weeks, up to about 85 weeks, up to about 86 weeks, up to about 87 weeks, up to about 88 weeks, up to about 89 weeks, up to about 90 weeks, up to about 91 weeks, about 92 weeks, up to about 93 weeks, up to about 94 weeks, up to about 95 weeks, up to about 96 weeks, up to about 97 weeks, up to about 98 weeks, up to about 100 weeks, up to about 105 weeks, up to about 110 weeks, up to about 115 weeks, up to about 120 weeks, up to about 125 weeks, up to about 130 weeks, up to about 135 weeks, up to about 140 weeks, up to about 145 weeks, up to about 150 weeks, up to about 155 weeks, up to about 160 weeks, up to about 165 weeks, up to about 170 weeks, up to about 175 weeks, up to about 180 weeks, up to about 185 weeks, up to about 190 weeks, up to about 195 weeks up, or to about 200 weeks. In some embodiments, the pharmaceutical composition comprising an antisense oligomer (e.g., eteplirsen), or pharmaceutically acceptable salt thereof, is administered for up to about 24 weeks. In some embodiments, the pharmaceutical composition comprising an antisense oligomer (e.g., eteplirsen), or pharmaceutically acceptable salt thereof, is administered for up to about 48 weeks. In some embodiments, the pharmaceutical composition comprising an antisense oligomer (e.g., eteplirsen), or pharmaceutically acceptable salt thereof, is administered for up to about 144 weeks. In some embodiments, the pharmaceutical composition comprising an antisense oligomer (e.g., eteplirsen), or pharmaceutically acceptable salt thereof, is administered for up to about 168 weeks.

In some embodiments, the methods of the disclosure comprise administering a pharmaceutical composition comprising an antisense oligomer (e.g., eteplirsen), or pharmaceutically acceptable salt thereof, wherein the composition is administered for at least about 24 weeks, at least about 25 weeks, at least about 26 weeks, at least about 27 weeks, at least about 28 weeks, at least about 29 weeks, at least about 30 weeks, at least about 31 weeks, at least about 32 weeks, at least about 33 weeks, at least about 34 weeks, at least about 35 weeks, at least about 36 weeks, at least about 37 weeks, at least about 38 weeks, at least about 39 weeks, at least about 40 weeks, at least about 41 weeks, at least about 42 weeks, at least about 43 weeks, at least about 44 weeks, at least about 45 weeks, at least about 46 weeks, at least about 47 weeks, at least about 48 weeks, at least about 49 weeks, at least about 50 weeks, at least about 51 weeks, at least about 52 weeks, at least about 53 weeks, at least about 54 weeks, at least about 55 weeks, at least about 56 weeks, at least about 57 weeks, at least about 58 weeks, at least about 59 weeks, at least about 60 weeks, at least about 61 weeks, at least about 62 weeks, at least about 63 weeks, at least about 64 weeks, at least about 65 weeks, at least about 66 weeks, at least about 67 weeks, at least about 68 weeks, at least about 69 weeks, at least about 70 weeks, at least about 71 weeks, about 72 weeks, at least about 73 weeks, at least about 74 weeks, at least about 75 weeks, at least about 76 weeks, at least about 77 weeks, at least about 78 weeks, at least about 79 weeks, at least about 80 weeks, at least about 81 weeks, at least about 82 weeks, at least about 83 weeks, at least about 84 weeks, at least about 85 weeks, at least about 86 weeks, at least about 87 weeks, at least about 88 weeks, at least about 89 weeks, at least about 90 weeks, at least about 91 weeks, about 92 weeks, at least about 93 weeks, at least about 94 weeks, at least about 95 weeks, at least about 96 weeks, at least about 97 weeks, at least about 98 weeks, at least about 100 weeks, at least about 105 weeks, at least about 110 weeks, at least about 115 weeks, at least about 120 weeks, at least about 125 weeks, at least about 130 weeks, at least about 135 weeks, at least about 140 weeks, at least about 145 weeks, at least about 150 weeks, at least about 155 weeks, at least about 160 weeks, at least about 165 weeks, at least about 170 weeks, at least about 175 weeks, at least about 180 weeks, at least about 185 weeks, at least about 190 weeks, at least about 195 weeks up, or to about 200 weeks. In some embodiments, the composition comprising an antisense oligomer (e.g., eteplirsen), or pharmaceutically acceptable salt thereof, is administered for at least 24 weeks, at least 36 weeks, at least 48 weeks, at least 120 weeks, at least 144 weeks, or at least 168 weeks. In yet other embodiments, the pharmaceutical composition is administered for the duration of the illness.

Some literature reports suggest an association in DMD patients between loss of ability to rise from the floor independently and a subsequent loss of ambulation within the following year (Bushby and Connor (2011) Clin Investig (Lond.) 1(9):1217-1235 and Henricson et al. (2013) Muscle Nerve 48(1):55-67). Accordingly, any of the methods described herein may involve treating a patient who has lost the ability to rise independently from supine. In some embodiments of any of the methods described herein, the patient loses the ability to rise independently from supine at least one year prior to treatment with an antisense oligomer (e.g., eteplirsen), or pharmaceutically acceptable salt thereof. In some embodiments of any of the methods described herein, the patient loses the ability to rise independently from supine within one year of beginning treatment with an antisense oligomer (e.g., eteplirsen), or pharmaceutically acceptable salt thereof. In some embodiments of any of the methods described herein, the patient loses the ability to rise independently from supine within two years of beginning treatment.

In some embodiments, any of the methods described herein comprise continuing treatment with an antisense oligomer (e.g., eteplirsen), or pharmaceutically acceptable salt thereof, even if a patient loses the ability to rise from a supine position during treatment with the antisense oligomer, or pharmaceutically acceptable salt thereof.

In some embodiments of any of the methods described herein, the patient has a rise time of greater than 10 seconds. In some embodiments of any of the methods described herein, the patient has a rise time of greater than 15 seconds. In some embodiments of any of the methods described herein, the patient has a rise time of greater than 20 seconds.

Clinical outcomes for analyzing the effect of the methods of the disclosure, wherein the antisense oligomer, or pharmaceutically acceptable salt thereof, induces exon 44, exon 45, exon 50, exon 51, exon 52, or exon 53 skipping, include percent dystrophin positive fibers (PDPF), six-minute walk test (6MWT), loss of ambulation (LOA), North Star Ambulatory Assessment (NSAA), pulmonary function tests (PFT), ability to rise (from a supine position) without external support, de novo dystrophin production, and other functional measures.

In some embodiments, the methods of the disclosure delay progression of the disease in the human subject treated with the methods, as measured by the 6 Minute Walk Test (6MWT).

In some embodiments, the methods of the disclosure allow to maintain pulmonary function or reducing loss of pulmonary function in a human subject treated with the methods. In some embodiments of any of the methods described herein, pulmonary function is measured as Maximum Expiratory Pressure (MEP). In some embodiments, pulmonary function is measured as Maximum Inspiratory Pressure (MIP). In some embodiments, pulmonary function is measured as Forced Vital Capacity (FVC).

In some embodiments, the methods of the disclosure restore an mRNA reading frame to induce dystrophin protein production in a human subject with DMD. Protein production can be measured by reverse-transcription polymerase chain reaction (RT-PCR), western blot analysis, or immunohistochemistry (IHC). In some embodiments, tissue from different muscle groups (e.g., the quadriceps, diaphragm, biceps, skin, heart, etc.) can be homogenized (for example, by the TissueLyser), isolated for RNA, and processed for PCR (for example, droplet digital PCR, “ddPCR”) to determine the levels of exon-skipping. In some embodiment, western blot analysis can be performed as described in Schnell et al., “Challenges in Interpreting Dystrophin Content by Western Blot,” US Neurology, 2019; 15(1):40-6, disclosure of which is incorporated herein in its entirety.

In some embodiments, the human subject to be treated by the methods of the disclosure is a male. In some embodiments, the human subject (e.g., male) is between about 6 months and about 4 years of age, inclusive. In some embodiments, the human subject is at least 6 (e.g., at least 7, 8, 9, 10, 11, 12, 13, 14, 15, 16, 17, 18, 19, 20, 24, 30, or 36) months of age. In some embodiments, the human subject is no greater than 4 years of age. In some embodiments, the male human subject is 7 to 13 years of age (inclusive). In other embodiments, the male human subject is younger than 7 years of age. In other embodiments, the male human subject is over the age of 13.

The methods of the disclosure also include administering a pharmaceutical composition comprising an antisense oligomer (e.g., eteplirsen), or pharmaceutically acceptable salt thereof, in combination with another therapeutic. The additional therapeutic may be administered prior, concurrently or subsequently to the administration of an antisense oligomer (e.g., eteplirsen), or pharmaceutically acceptable salt thereof. The additional therapeutic may be formulated in the same composition as the antisense oligomer (e.g., eteplirsen), or pharmaceutically acceptable salt thereof, or may be in a different composition. For example, an antisense oligomer (e.g., eteplirsen), or pharmaceutically acceptable salt thereof, may be administered in combination with a steroid and/or an antibiotic. The steroid may be a glucocorticoid or prednisone. Other agents which can be administered include an antagonist of the ryanodine receptor, such as dantrolene, which has been shown to enhance antisense-mediated exon skipping in patient cells and a mouse model of DMD (G. Kendall et al. Sci Tranl Med 4 164ra160 (2012), incorporated herein by reference).

In some embodiments, the methods of the disclosure include co-administering an antisense oligomer (e.g., eteplirsen), or pharmaceutically acceptable salt thereof, either per se or as a pharmaceutical composition, with a carbohydrate, either in the same composition or is a separate composition, as provided in Han et al., Nat. Comms. 7, 10981 (2016) the entirety of which is incorporated herein by reference. In some embodiments, an antisense oligomer (e.g., eteplirsen), or pharmaceutically acceptable salt thereof, may be co-administered with 5% of a hexose carbohydrate. For example, an antisense oligomer (e.g., eteplirsen), or pharmaceutically acceptable salt thereof, may be co-administered with 5% glucose, 5% fructose, or 5% mannose. In certain embodiments, an antisense oligomer (e.g., eteplirsen), or pharmaceutically acceptable salt thereof, may be co-administered with 2.5% glucose and 2.5% fructose. In some embodiments, an antisense oligomer (e.g., eteplirsen), or pharmaceutically acceptable salt thereof, may be co-administered with a carbohydrate selected from: arabinose present in an amount of 5% by volume, glucose present in an amount of 5% by volume, sorbitol present in an amount of 5% by volume, galactose present in an amount of 5% by volume, fructose present in an amount of 5% by volume, xylitol present in an amount of 5% by volume, mannose present in an amount of 5% by volume, a combination of glucose and fructose each present in an amount of 2.5% by volume, and a combination of glucose present in an amount of 5.7% by volume, fructose present in an amount of 2.86% by volume, and xylitol present in an amount of 1.4% by volume.

In various embodiments, an antisense oligomer (e.g., eteplirsen), or pharmaceutically acceptable salt thereof, either per se or as pharmaceutical composition, is co-administered with a therapeutically effective amount of a non-steroidal anti-inflammatory compound. In some embodiments, the non-steroidal anti-inflammatory compound is an NF-kB inhibitor. For example, in some embodiments, the NF-kB inhibitor may be CAT-1004 or a pharmaceutically acceptable salt thereof. In various embodiments, the NF-kB inhibitor may be a conjugate of salicylate and DHA. In some embodiments, the NF-kB inhibitor is CAT-1041 or a pharmaceutically acceptable salt thereof. In certain embodiments, the NF-kB inhibitor is a conjugate of salicylate and EPA. In various embodiments, the NF-kB inhibitor is

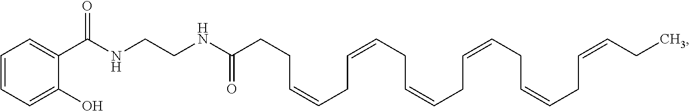 [SMILES: CC/C=C\C/C=C\C/C=C\C/C=C\C/C=C\C/C=C\CCC(=O)NCCNC(=O)c1ccccc1O]

or a pharmaceutically acceptable salt thereof.

In some embodiments, non-steroidal anti-inflammatory compound is a TGF-b inhibitor. For example, in certain embodiments, the TGF-b inhibitor is HT-100.

### III. Formulations

Methods according to the disclosure include administration of an antisense oligomer (e.g., eteplirsen), or pharmaceutically acceptable salt thereof, as a pharmaceutical composition comprising the antisense oligomer, or pharmaceutically acceptable salt thereof. Methods for the delivery of nucleic acid molecules are described, for example, in Akhtar et al., 1992, Trends Cell Bio., 2:139; and Delivery Strategies for Antisense Oligonucleotide Therapeutics, ed. Akhtar; Sullivan et al., PCT WO 94/02595. These and other protocols can be utilized for the delivery of virtually any nucleic acid molecule, including eteplirsen.

In certain embodiments, the disclosure provides methods comprising administering a pharmaceutically acceptable compositions that comprise a therapeutically-effective amount of an antisense oligomer (e.g., eteplirsen), or pharmaceutically acceptable salt thereof, formulated together with one or more pharmaceutically acceptable carriers (additives) and/or diluents.

In some embodiments, the pharmaceutical composition comprises from about 0.1 to about 99% of an antisense oligomer (e.g., eteplirsen), or pharmaceutically acceptable salt thereof, together with a pharmaceutically acceptable excipient. In some embodiments, the pharmaceutical composition comprises from about 1 to about 90% of an antisense oligomer (e.g., eteplirsen), or pharmaceutically acceptable salt thereof. In some embodiments, the pharmaceutical composition comprises from about 5 to about 70%, about 5 to about 60%, about 5 to about 50%, about 5 to about 40%, or about 5 to about 30% of an antisense oligomer (e.g., eteplirsen), or pharmaceutically acceptable salt thereof. In other embodiments, the pharmaceutical composition comprises from about 10 to about 80%, about 10 to about 70%, about 10 to about 60%, about 10 to about 50%, about 10 to about 40%, about 10 to about 30%, or about 10 to about 20% of an antisense oligomer (e.g., eteplirsen), or pharmaceutically acceptable salt thereof. In other embodiments, the concentration of an antisense oligomer (e.g., eteplirsen), or pharmaceutically acceptable salt thereof, in the pharmaceutical composition is about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, or about 200 mg/ml. In one embodiment, the concentration of an antisense oligomer (e.g., eteplirsen), or pharmaceutically acceptable salt thereof, in the pharmaceutical composition is about 50 mg/ml.

The pharmaceutical compositions suitable for administration in the methods of the disclosure may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Some examples of materials that can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Additional non-limiting examples of agents suitable for formulation with an antisense oligomer (e.g., eteplirsen), or pharmaceutically acceptable salt thereof, include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues; biodegradable polymers, such as poly (DL-lactide-coglycolide) microspheres for sustained release delivery after implantation (Emerich, D F et al., 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

The disclosure also features the use of the composition comprising surface-modified liposomes containing poly (ethylene glycol) lipids (PEG-modified, branched and unbranched or combinations thereof, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

In a further embodiment, the disclosure includes an antisense oligomer (e.g., eteplirsen), or pharmaceutically acceptable salt thereof, compositions prepared for delivery as described in U.S. Pat. Nos. 6,692,911, 7,163,695 and 7,070,807. In this regard, in one embodiment, the disclosure provides an antisense oligomer (e.g., eteplirsen), or pharmaceutically acceptable salt thereof, in a composition comprising copolymers of lysine and histidine (HK) (as described in U.S. Pat. Nos. 7,163,695, 7,070,807, and 6,692,911) either alone or in combination with PEG (e.g., branched or unbranched PEG or a mixture of both), in combination with PEG and a targeting moiety or any of the foregoing in combination with a crosslinking agent. In certain embodiments, the disclosure provides eteplirsen in compositions comprising gluconic-acid-modified polyhistidine or gluconylated-polyhistidine/transferrin-polylysine. One skilled in the art will also recognize that amino acids with properties similar to His and Lys may be substituted within the composition.

In some embodiments, an antisense oligomer (e.g., eteplirsen) can be administered as a pharmaceutically acceptable salt. The term “pharmaceutically-acceptable salts” in this respect, refers to the relatively non-toxic, inorganic and organic acid and base addition salts of an antisense oligomer. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19). The pharmaceutically acceptable salts also include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. Representative alkali or alkaline earth salts include the lithium, sodium, potassium, calcium, magnesium, and aluminum salts and the like, are also included, as well as representative organic amines useful for the formation of base addition salts include ethylamine, diethylamine, ethylenediamine, ethanolamine, diethanolamine, piperazine and the like. (See, e.g., Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for use in the methods of the disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

Methods of preparing pharmaceutical compositions comprising an antisense oligomer (e.g., eteplirsen), or pharmaceutically acceptable salt thereof, include the step of bringing into association an antisense oligomer (e.g., eteplirsen), or pharmaceutically acceptable salt thereof, with the carrier and, optionally, one or more accessory ingredients. In general, the formulations are prepared by uniformly and intimately bringing an antisense oligomer (e.g., eteplirsen), or pharmaceutically acceptable salt thereof, into association with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an antisense oligomer (e.g., eteplirsen), or pharmaceutically acceptable salt thereof, as an active ingredient. An antisense oligomer (e.g., eteplirsen), or pharmaceutically acceptable salt thereof, may also be administered as a bolus, electuary or paste.

In solid dosage forms of the disclosure for oral administration (capsules, tablets, pills, dragees, powders, granules, trouches and the like), an antisense oligomer (e.g., eteplirsen), or pharmaceutically acceptable salt thereof, may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (e.g., gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of an antisense oligomer (e.g., eteplirsen), or pharmaceutically acceptable salt thereof, include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing an antisense oligomer (e.g., eteplirsen), or pharmaceutically acceptable salt thereof, with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations or dosage forms for the topical or transdermal administration of an antisense oligomer (e.g., eteplirsen), or pharmaceutically acceptable salt thereof, include powders, sprays, ointments, pastes, creams, lotions, gels, solutions, patches and inhalants. An antisense oligomer (e.g., eteplirsen), or pharmaceutically acceptable salt thereof, may be mixed under sterile conditions with a pharmaceutically-acceptable carrier, and with any preservatives, buffers, or propellants which may be required. The ointments, pastes, creams and gels may contain, in addition to an antisense oligomer (e.g., eteplirsen), or pharmaceutically acceptable salt thereof, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an antisense oligomer (e.g., eteplirsen), or pharmaceutically acceptable salt thereof, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of an antisense oligomer (e.g., eteplirsen), or pharmaceutically acceptable salt thereof, to the body. Such dosage forms can be made by dissolving or dispersing the oligomer in the proper medium. Absorption enhancers can also be used to increase the flux of the agent across the skin. The rate of such flux can be controlled by either providing a rate controlling membrane or dispersing the agent in a polymer matrix or gel, among other methods known in the art.

Pharmaceutical compositions suitable for parenteral administration may comprise an antisense oligomer (e.g., eteplirsen), or pharmaceutically acceptable salt thereof, in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In one embodiment, the pharmaceutical composition comprises a phosphate-buffered saline.

The pharmaceutical compositions may also contain adjuvants such as preservatives, wetting agents, emulsifying agents and dispersing agents. Prevention of the action of microorganisms upon the subject oligomers may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility, among other methods known in the art. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms may be made by forming microencapsule matrices of an antisense oligomer (e.g., eteplirsen), or pharmaceutically acceptable salt thereof, in biodegradable polymers such as polylactide-polyglycolide. Depending on the ratio of an antisense oligomer (e.g., eteplirsen), or pharmaceutically acceptable salt thereof, to polymer, and the nature of the particular polymer employed, the rate of release can be controlled. Examples of other biodegradable polymers include poly(orthoesters) and poly(anhydrides). Depot injectable formulations may also prepared by entrapping the drug in liposomes or microemulsions that are compatible with body tissues.

An antisense oligomer (e.g., eteplirsen), or pharmaceutically acceptable salt thereof, can be administered to cells by a variety of methods known to those familiar to the art, including, but not restricted to, iontophoresis, or by incorporation into other vehicles, such as hydrogels, cyclodextrins, biodegradable nanocapsules, and bioadhesive microspheres, as described herein and known in the art. In certain embodiments, microemulsification technology may be utilized to improve bioavailability of lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other benefits, microemulsification provides enhanced bioavailability by preferentially directing absorption to the lymphatic system instead of the circulatory system, which thereby bypasses the liver, and prevents destruction of the compounds in the hepatobiliary circulation.

Cyclodextrins are cyclic oligosaccharides, consisting of 6, 7 or 8 glucose units, designated by the Greek letter α, β, or γ, respectively. The glucose units are linked by α-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17α-estradiol (see, e.g., van Uden et al. Plant Cell Tiss. Org. Cult. 38:1-3-113 (1994)). The complexation takes place by Van der Waals interactions and by hydrogen bond formation. For a general review of the chemistry of cyclodextrins, see, Wenz, Agnew. Chem. Int. Ed. Engl., 33:803-822 (1994).

The physico-chemical properties of the cyclodextrin derivatives depend strongly on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259) and Gramera, et al. (U.S. Pat. No. 3,459,731) described electroneutral cyclodextrins. Other derivatives include cyclodextrins with cationic properties [Parmeter (II), U.S. Pat. No. 3,453,257], insoluble crosslinked cyclodextrins (Solms, U.S. Pat. No. 3,420,788), and cyclodextrins with anionic properties [Parmeter (III), U.S. Pat. No. 3,426,011]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127).

In one aspect, the formulations contain micelles formed from an antisense oligomer (e.g., eteplirsen), or pharmaceutically acceptable salt thereof, and at least one amphiphilic carrier, in which the micelles have an average diameter of less than about 100 nm. More preferred embodiments provide micelles having an average diameter less than about 50 nm, and even more preferred embodiments provide micelles having an average diameter less than about 30 nm, or even less than about 20 nm.

While all suitable amphiphilic carriers are contemplated, the presently preferred carriers are generally those that have Generally-Recognized-as-Safe (GRAS) status, and that can both solubilize the compound of the disclosure and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Examples of amphiphilic carriers include saturated and monounsaturated polyethyleneglycolyzed fatty acid glycerides, such as those obtained from fully or partially hydrogenated various vegetable oils. Such oils may advantageously consist of tri-, di-, and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers may be particularly useful, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc (produced and distributed by a number of companies in USA and worldwide).

In certain embodiments, the delivery of an antisense oligomer (e.g., eteplirsen), or pharmaceutically acceptable salt thereof, may occur by use of liposomes, nanocapsules, microparticles, microspheres, lipid particles, vesicles, and the like, for the introduction of the compositions of the disclosure into suitable host cells. In particular, the compositions of the disclosure may be formulated for delivery either encapsulated in a lipid particle, a liposome, a vesicle, a nanosphere, a nanoparticle or the like. The formulation and use of such delivery vehicles can be carried out using known and conventional techniques.

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

One aspect of the disclosure relates to formulations comprising liposomes containing an antisense oligomer (e.g., eteplirsen), or pharmaceutically acceptable salt thereof, where the liposome membrane is formulated to provide a liposome with increased carrying capacity. Alternatively or in addition, an antisense oligomer (e.g., eteplirsen), or pharmaceutically acceptable salt thereof, may be contained within, or adsorbed onto, the liposome bilayer of the liposome. An antisense oligomer (e.g., eteplirsen), or pharmaceutically acceptable salt thereof, may be aggregated with a lipid surfactant and carried within the liposome's internal space; in these cases, the liposome membrane is formulated to resist the disruptive effects of the active agent-surfactant aggregate.

According to one embodiment of the disclosure, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Aggregates of surfactant and active agent (such as emulsions or micelles containing the active agent of interest) may be entrapped within the interior space of liposomes to disperse and/or solubilize an antisense oligomer (e.g., eteplirsen), or pharmaceutically acceptable salt thereof. A surfactant may be selected from any suitable aliphatic, cycloaliphatic or aromatic surfactant, including but not limited to biocompatible lysophosphatidylcholines (LPGs) of varying chain lengths (for example, from about C14 to about C20). Polymer-derivatized lipids such as PEG-lipids may also be utilized for micelle formation as they will act to inhibit micelle/membrane fusion, and as the addition of a polymer to surfactant molecules decreases the CMC of the surfactant and aids in micelle formation. Preferred are surfactants with CMOs in the micromolar range; higher CMC surfactants may be utilized to prepare micelles entrapped within liposomes of the disclosure.

Liposomes according to the disclosure may be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic D D, Liposomes from physics to applications, Elsevier Science Publishers BV, Amsterdam, 1993. For example, liposomes of the disclosure may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, an antisense oligomer (e.g., eteplirsen), or pharmaceutically acceptable salt thereof, is first dispersed by sonication in a lysophosphatidylcholine or other low CMC surfactant (including polymer grafted lipids) that readily solubilizes hydrophobic molecules. The resulting micellar suspension of an antisense oligomer (e.g., eteplirsen), or pharmaceutically acceptable salt thereof, is then used to rehydrate a dried lipid sample that contains a suitable mole percent of polymer-grafted lipid, or cholesterol. The lipid and an antisense oligomer suspension is then formed into liposomes using extrusion techniques as are known in the art, and the resulting liposomes separated from the unencapsulated solution by standard column separation.

In one aspect of the disclosure, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988). In certain embodiments, reagents such as DharmaFECT® and Lipofectamine® may be utilized to introduce polynucleotides or proteins into cells.

The release characteristics of a formulation of the disclosure depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In most cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween® and Pluronic®. Pore forming agents which add microstructure to the matrices (i.e., water soluble compounds such as inorganic salts and sugars) are added as particulates. The range is typically between one and thirty percent (w/w polymer).

Hydrophilic polymers suitable for use in the liposomes are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. In certain embodiments, polymers have a molecular weight of from about 100 or 120 daltons up to about 5,000 or 10,000 daltons, or from about 300 daltons to about 5,000 daltons. In other embodiments, the polymer is polyethyleneglycol having a molecular weight of from about 100 to about 5,000 daltons, or having a molecular weight of from about 300 to about 5,000 daltons. In certain embodiments, the polymer is polyethyleneglycol of 750 daltons (PEG(750)). Polymers may also be defined by the number of monomers therein; a preferred embodiment of the disclosure utilizes polymers of at least about three monomers, such PEG polymers consisting of three monomers (approximately 150 daltons).

Other hydrophilic polymers which may be suitable for use in the disclosure include polyvinylpyrrolidone, polymethoxazoline, polyethyloxazoline, polyhydroxypropyl methacrylamide, polymethacrylamide, poly dimethylacrylamide, and derivatized celluloses such as hydroxymethylcellulose or hydroxyethylcellulose.

In certain embodiments, a formulation of the disclosure comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

An antisense oligomer (e.g., eteplirsen), or pharmaceutically acceptable salt thereof, may be formulated to be contained within, or, adapted to release by a surgical or medical device or implant. In certain aspects, an implant may be coated or otherwise treated with an oligomer. For example, hydrogels, or other polymers, such as biocompatible and/or biodegradable polymers, may be used to coat an implant with the compositions of the disclosure (i.e., the composition may be adapted for use with a medical device by using a hydrogel or other polymer). Polymers and copolymers for coating medical devices with an agent are well-known in the art. Examples of implants include, but are not limited to, stents, drug-eluting stents, sutures, prosthesis, vascular catheters, dialysis catheters, vascular grafts, prosthetic heart valves, cardiac pacemakers, implantable cardioverter defibrillators, IV needles, devices for bone setting and formation, such as pins, screws, plates, and other devices, and artificial tissue matrices for wound healing.

The routes of administration described are intended only as a guide since a skilled practitioner will be able to determine readily the optimum route of administration. Multiple approaches for introducing functional new genetic material into cells, both in vitro and in vivo have been attempted (Friedmann (1989) Science, 244:1275-1280). These approaches include integration of the gene to be expressed into modified retroviruses (Friedmann (1989) supra; Rosenberg (1991) Cancer Research 51(18), suppl.: 5074S-5079S); integration into non-retrovirus vectors (e.g., adeno-associated viral vectors) (Rosenfeld, et al. (1992) Cell, 68:143-155; Rosenfeld, et al. (1991) Science, 252:431-434); or delivery of a transgene linked to a heterologous promoter-enhancer element via liposomes (Friedmann (1989), supra; Brigham, et al. (1989) Am. J. Med. Sci., 298:278-281; Nabel, et al. (1990) Science, 249:1285-1288; Hazinski, et al. (1991) Am. J. Resp. Cell Molec. Biol., 4:206-209; and Wang and Huang (1987) Proc. Natl. Acad. Sci. (USA), 84:7851-7855); coupled to ligand-specific, cation-based transport systems (Wu and Wu (1988) J. Biol. Chem., 263:14621-14624) or the use of naked DNA, expression vectors (Nabel et al. (1990), supra); Wolff et al. (1990) Science, 247:1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group (Am. J. Med. Sci. (1989) 298:278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, Science (1992) 256:808-813.

### EXAMPLES

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### Example 1. High-Dose Eteplirsen Treatment in the hDMD Δ52 mdx Mouse Model

Eteplirsen was tested in a humanized DMD mdx mouse model, wherein the human DMD with exon 52 deletion-YAC transgene (hDMD Δ52 mouse) was integrated into mouse chromosome 5. The model is described in Hoen et al., “Generation and Characterization of Transgenic Mice with the Full-length Human DMD Gene,” J. Biol. Chem., 2008, 283(9):5899-5907, disclosure of which is incorporated herein in its entirety.

Deletion of exon 52 in the human DMD gene produces an out of frame pre-mRNA. The hDMD Δ52 mouse is amenable to exon 51 skipping drugs. As demonstrated for DMD patients, eteplirsen is designed to bind to exon 51 of dystrophin pre-mRNA in the hDMD Δ52 mouse, resulting in exclusion of this exon during mRNA processing. Skipping of exon 51 by eteplirsen is anticipated to restore the open reading frame of dystrophin mRNA during protein translation. This is intended to allow for the production of an internally shortened dystrophin protein that localizes to the muscle membrane and can protect muscle from damage, as well as improve force in dystrophic muscle fibres.

### Experimental Design

hDMD Δ52 mice were treated with once weekly (QW) intravenous injections of vehicle or eteplirsen for 1 (500 or 960 mg/kg), 4 (500 or 750 mg/kg), and 8 weeks (500 or 750 mg/kg) or 8 weeks. The plasma exposures at 500, 750, and 960 mg/kg doses in mice are equivalent to 100, 150, and 192 mg/kg plasma exposures in humans. This is based on 5:1 dose/exposure relationship difference between mouse and human.

### Results

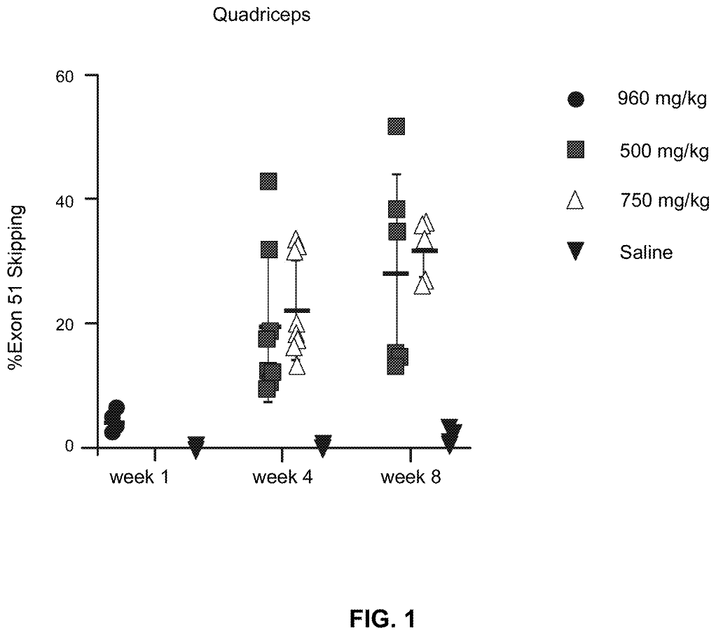

Exon 51 skipping, dystrophin production, and grip test results in hDMD Δ52 mice after 4 and 8 once weekly administration of eteplirsen at exposures in the same range as 100-200 mg/kg in patients is shown in FIGS. 1-3.

Percent exon 51 skipping in hDMD Δ52 mdx mice (n=6) was measured using droplet digital PCR (ddPCR). Briefly, the quadriceps, diaphragm, biceps, skin and heart were homogenized by the TissueLyser, isolated for RNA, and processed for ddPCR to determine the levels of exon-skipping. FIG. 1 shows that eteplirsen increases % exon 51 skipping in hDMD Δ52 mice.

Dystrophin production in hDMD Δ52 mdx mice (n=6) was measured according to the following method. Protein lysates prepared in NCH buffer were diluted to 0.2 μg/μ1 in 0.1× sample buffer provided with the Jess kit. A wild type standard curve was prepared using pooled wild type lysates diluted in pooled dystrophic lysate, at a concentration of 0.2 μg/μl in 0.1× sample buffer. 4 μl of each sample and 1 μl of prepared fluorescent 5× master mix were added to a PCR plate. The plate was then incubated at 95° C. for 5 minutes, and then placed on ice. 3W sample was then loaded onto the Jess plate, in the row indicated by the manufacturer. The rest of the plate was prepared according to the manufacturer's instructions, using milk free antibody diluent.

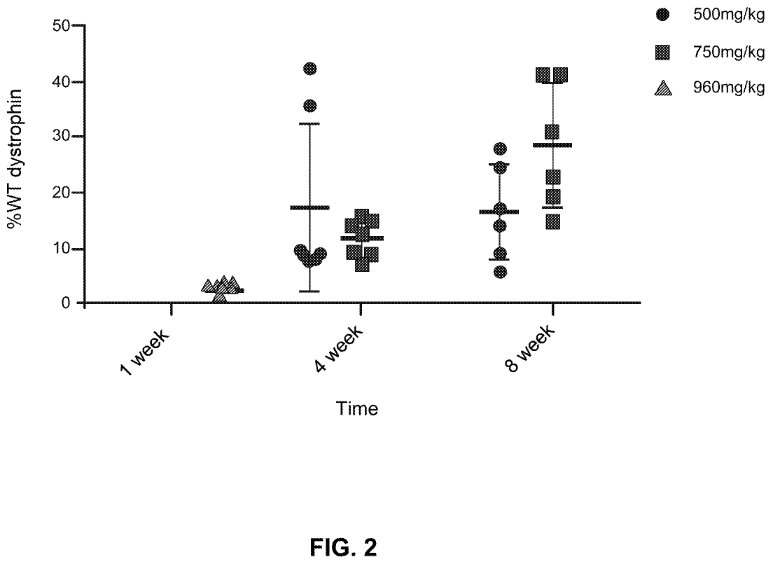

The primary antibody cocktail was prepared by diluting dystrophin antibody (ab154168) 1:1000, and α-actinin (ab254074) 1:50 together in milk-free antibody diluent. The secondary antibody cocktail was prepared by diluting 20×NIR anti-mouse antibody (obtained from Protein Simple; Beekman et al., “Use of capillary Western immunoassay (Wes) for quantification of dystrophin levels in skeletal muscle of healthy controls and individuals with Becker and Duchenne muscular dystrophy,” PLOS One, Apli 11, 2018, content of which is incorporated herein in its entirety) in anti-rabbit chemiluminescent antibody (Protein simple). Once the plate was loaded, it was placed in the Jess, and a 66-440 kDa 25 capillary module was inserted into the instrument. The assay was then run using the programs for both chemiluminescence and fluorescence. After the run, dystrophin was normalized to actinin by dividing the peak area for dystrophin (308 kDA) by the peak area for actinin (106 kDa) for each well. Percent WT was calculated by fitting a line to the standard curve and using the equation of that line to find the percent of dystrophin compared to the humanized DMD WT mouse. FIG. 2 demonstrates that eteplirsen increases dystrophin production in hDMD Δ52 mdx mice.

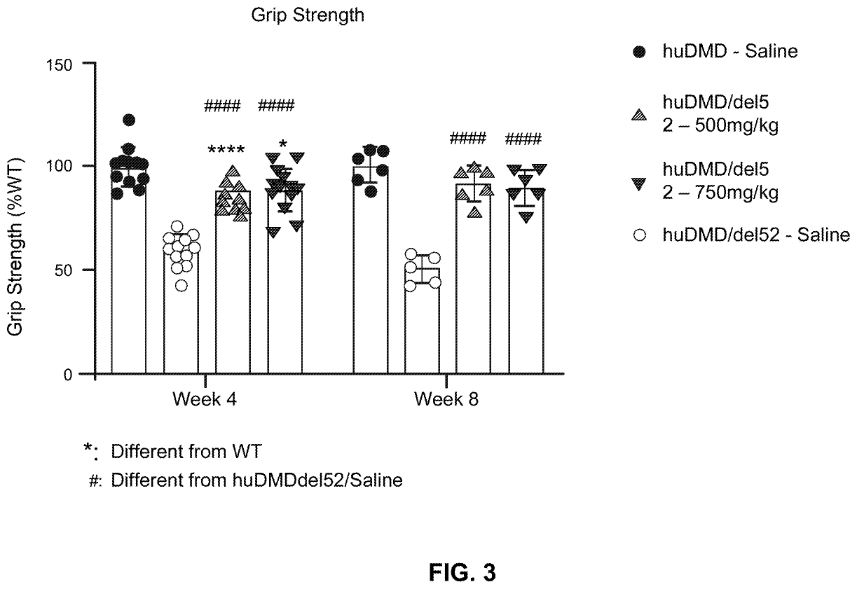

The Grip Strength test measures the maximal peak force developed by a rodent. The peak force is measured in grams and is obtained by the operator drawing the mouse along a straight line over a grid leading away from the sensor it is attached to. The animal will release at the end of the grid and a maximum force measurement is obtained. Three measurements are taken per each day of testing and averaged to obtain results. Results are normalized to the animals bodyweight. Mice are tested at onset of study to determine a baseline and train mice to the apparatus. FIG. 3 shows that eteplirsen improves function in hDMD Δ52 mdx mice.

### Example 2. Safety and Efficacy of High-Dose Eteplirsen in Non-Human Primates

This example evaluates safety and efficacy of eteplirsen administered either intravenously (IV) or subcutaneously (SC) to cynomolgus monkeys once weekly over 12 weeks, at doses of 5, 40, and 320 mg/kg per injection.

### Experimental Design

A total of 60, male and female cynomolgus monkeys were used in the study, with 12 animals per each dose group (6 males and 6 females), as show below. All animals received either intravenous bolus injection (IV) infusions or subcutaneous injection (SC) weekly for 12 weeks, then pharmacodynamics (PD), as represented by exon skipping, was assessed at the end of the study.

Group 1 animals were administered Vehicle Control. Groups 2-4 were administered eteplirsen at dose levels of 5-320 mg/kg as IV bolus. Group 5 was administered eteplirsen as SC dose at 320 mg/kg dose. A 320 mg/kg dose in humans is predicted to provide AUC and Cₘₐₓ PK that are equivalent to the AUC and Cₘₐₓ PK provided by 320 mg/kg in NHP. Based on the dose-linear exposure relationship observed in NHP, the human 200 mg/kg dose is expected to exhibit similar AUC and Cₘₐₓ PK.

Necropsy and Biopsy Schedule

| | | Dose | Dose | |
|---|---|---|---|---|
| Group | Route of | Level | volume | Number of |
| No. | administration | (mg/kg) | (mL/kg) | animals |
| 1 | IV Bolus | 0 | 3.2 | 12 |
| 2 | IV Bolus | 5 | 2.5 | 12 |
| 3 | IV Bolus | 40 | 2.0 | 12 |
| 4 | IV Bolus | 320 | 3.2 | 12 |
| 5 | subcutaneous | 320 | 3.2 | 12 |
| | injection | | | |

All animals were monitored throughout the study with clinical observations and body weight measurements. Clinical pathology evaluations on blood samples were conducted on all animals pretest, during week 4, and prior to the terminal and recovery necropsies. Urine samples for clinical pathology evaluations were collected from all animals pretest and prior to the terminal and recovery necropsies. Blood samples (approximately 4.8 to 5.8 mL) were collected from the femoral vein. Samples were collected into tubes containing K3EDTA for evaluation of hematology parameters and sodium citrate for evaluation of coagulation parameters. A serum separator was used for the clinical chemistry samples. Urine samples were collected using steel pans placed under the cages for approximately 16 hours.

### Results

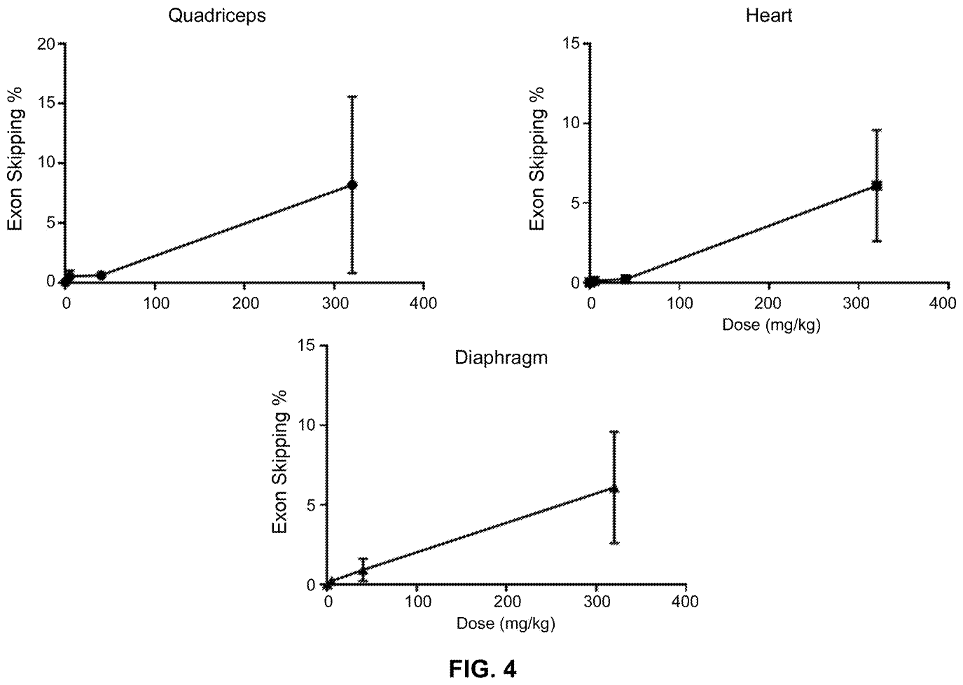

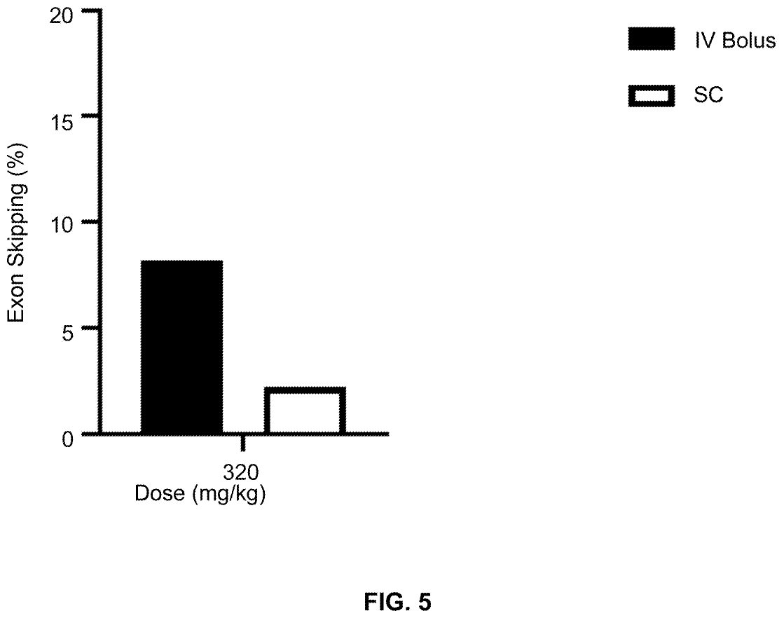

The quad, heart, and diaphragm tissues were homogenized and processed for RT-PCR analysis to determine the levels of exon 51 skipping. No exon skipping was detected from the vehicle treated groups. Exon skipping was detected at all doses in the three muscle groups, as shown in FIG. 4. A dose-dependent increase in exon skipping was observed in quadriceps, heart and diaphragm. Both IV and SC administrations resulted in measurable exon skipping levels, as demonstrated in FIG. 5. The bioavailability of the SC administration on Day 1 was 104%.

In conclusion, administration of eteplirsen by intravenous infusion for 12 doses was clinically well tolerated under the conditions of this study in cynomolgus monkeys. Based on the absence of adverse findings, the No-Observed-Adverse-Effect-Level for AVI-4658 in male and female cynomolgus monkeys administered 12 weekly IV bolus doses was 320 mg/kg (IV and SC) corresponding to an average AUC of 1020 hr*mg/mL (IV) and 1250 hr*mg/mL (SC) at day 71 of the study. The bioavailability of the SC administration on Day 1 is 104%, which demonstrates that both IV and SC administration can be used to administer eteplirsen. Both IV and SC administrations resulted in measurable exon skipping levels.

### Example 3. Treatment of Human Patients with High-Dose Eteplirsen

### Patients

Eligible patients are boys between 7 and 13 years of age (inclusive), with out-of-frame deletions of the DMD gene that could be corrected by skipping exon 51. Patients have achieved a mean 6-minute walk test (6MWT) distance of ≥300 and ≤450 meters (without assistance) at both the screening and baseline visits. Patients have intact right and left biceps muscles (the preferred biopsy site) or an alternative upper arm muscle group that will allow for sufficiently sized (1 cm³) muscle biopsies to be obtained prior to and on treatment. Patient also have been on a stable dose or dose equivalent of oral corticosteroids for at least 24 weeks prior to randomization, and the dose is expected to remain constant (except for modifications to accommodate changes in weight) throughout the study. Patients also have stable pulmonary function (forced vital capacity≥50% of predicted and no requirement for nocturnal ventilation) and pulmonary function is unlikely to decompensate significantly over the duration of the study.

### Study Drug

Eteplirsen [sequence 5′-CTCCAACATCAAGGAAGATGGCATTTCTAG-3′] (SEQ ID NO:1) is supplied by Sarepta Therapeutics, Inc. in single-use vials of phosphate-buffered saline (100 mg/ml). Eteplirsen is reconstituted with 150 ml normal saline and infused over 60 minutes. Placebo is administered during the first 24 weeks of the study. Placebo is supplied as identical vials of phosphate-buffered saline and administered in the same manner as eteplirsen.

### Study Design

### Open-Label Dose Escalation:

The objective of this study is to evaluate the safety and tolerability of weekly IV doses of 100 and 200 mg/kg of eteplirsen, and to evaluate the pharmacokinetics of 100 and 200 mg/kg of eteplirsen.

A cohort of 4 eligible DMD patients is treated with eteplirsen IV weekly at a dose of 100 mg/kg followed by 200 mg/kg, for 4 weeks at each dose level; each 4-week treatment period may be extended based on patient enrollment. After each 2-week treatment period has been completed by 4 patients, safety and tolerability data is reviewed to determine if the patients will continue dosing for 2 more weeks. The cohort may be expanded by 2 additional patients at each dose level, with initiation of dosing after 2 weeks of treatment within the applicable 4-week treatment period. The available data is evaluated to determine if 100 mg/kg or both 100 and 200 mg/kg weekly IV doses is (are) adequately safe and tolerated in DMD patients such that the Double-blind portion of the study can begin. Patients in the Open-label Dose Escalation continue treatment with the selected high dose as a distinct cohort.

### Double-Blind:

This is a randomized, double-blind, dose finding and dose comparison evaluation of the safety and efficacy of 1 or 2 higher doses of eteplirsen, 100 mg/kg and 200 mg/kg, and 30 mg/kg, administered once weekly IV, in approximately 114 Duchenne muscular dystrophy (DMD) patients with genetically confirmed deletion mutations amenable to treatment by skipping exon 51. Randomization is stratified by baseline NSAA total score (≤22 or ≥22). Patients are randomized in a 1:1:1 ratio into 3 dose groups: 30, 100 and 200 mg/kg, provided that the latter 2 doses are determined to be safe and tolerable in the Open-label Dose Escalation. At the completion of Dose Finding, patients who received either 100 mg/kg or 200 mg/kg continue on the selected high dose while those previously randomized to 30 mg/kg will continue on that dose.

If only the 100 mg/kg dose is deemed acceptable in the Dose Escalation, Dose Finding is not conducted, and patients are randomized in a 1:2 ratio to 30 or 100 mg/kg doses for Dose Comparison.

All patients undergo baseline muscle biopsy pre-dose. Each patient undergoes one additional muscle biopsy at either 12, 24, or 48 weeks (24 patients at Week 12, 45 patients at Week 24, and 45 patients at Week 48). Interim analyses is performed on muscle biopsy data at Week 12 to assess dystrophin expression for high dose selection; additional interim analyses is conducted at Week 24 and, if a high dose is not selected, at Week 48. Once a high dose is selected, at either Week 12, 24 or 48, patients enter the Dose Comparison part of the study; if interim analyses to select a high dose are not necessary at Week 24 or Week 48, the assessments for dystrophin expression at those weeks is conducted as part of Dose Comparison. Both PD (dystrophin expression endpoints) and safety endpoints is taken into account for high dose selection.

### Interim Analysis

To enable high dose selection between 100 mg/kg and 200 mg/kg during Double-blind part, interim analyses is performed on muscle biopsy data from Weeks 12, 24 and 48 to assess dystrophin expression after the scheduled muscle biopsy tissues at these time points have been collected and measured for dystrophin expression using Western blot, as described above. To preserve study integrity, unblinded muscle biopsy interim analysis is performed by an independent external unblinded statistical group. The dose selection decision occurs at any of the muscle biopsy interim analyses. If a dose selection decision occurs at an earlier time point (i.e., Week 12 or Week 24), subsequent muscle biopsy interim analysis(es) is conducted for dose comparison on dystrophin expression endpoints.

### Example 4. DMD Patient Myotube Assay with High-Dose Golodirsen

The pharmacological activity of high-dose golodirsen was assessed in a well characterized in vitro cellular model of Duchenne muscular dystrophy.

### Experimental Design

The cell model used is a muscle cell line isolated from a DMD patient with an exon 52 deletion (DMD Del52) and immortalized by the Institute of Myology using a method shown to preserve the essential primary skeletal muscle characteristics of the cells (Mamchaoui et al., “Immortalized pathological human myoblasts: towards a universal tool for the study of neuromuscular disorders,” Skeletal Muscle, 2011, 1(1):34; Thorley et al., “Skeletal muscle characteristics are preserved in hTERT/cdk4 human myogenic cell lines,” Skeletal Muscle, 2016, 6(1):43; content of both references is incorporated herein in its entirety).

### Cell Lines and Culturing Condition

Myoblasts were isolated from the paravertebral muscles of a 16 year old healthy donor and 16 year old DMD patient with a deletion in exon 52 and immortalized by the Institute of Myology by ectopic expression of hTERT and CDK4 as previously described (Mamchaoui et al., “Immortalized pathological human myoblasts: towards a universal tool for the study of neuromuscular disorders,” Skeletal Muscle, 2011, 1(1):34). Cells were maintained in proliferation medium containing 1 volume medium 199, 4 volumes Dulbecco's modified Eagle's medium (DMEM), 20% fetal bovine serum, 50 μg/ml gentamycin, 25 μg/ml fetuin, 0.5 μg/ml bFGF, 5 ng/ml EGF, 0.2 μg/ml dexamethasone, 5 μg/ml insulin on tissue culture plates coated with 1% collagen I and 0.5% MaxGel (Sigma-Aldrich E0282) at 50 ul/cm2 for 3 hr at 37° C.

### In Vitro Assay

The golodirsen lot used was synthesized at Bachem and purity was assessed at 92%. Positive and negative control compounds were included on each plate for assay quality control. The positive control was 30 μM SRP-5051, lot #RD00128-17 and the negative control 30 μM RC-1001 (MZ-194-170). All compounds were dissolved in sterile water and the concentration was confirmed by spectrophotometry prior to assay. Myoblasts were plated in proliferation medium at 6000 cells/well in a 96-well clear bottom imaging plate (Perkin Elmer #6055300) coated with 1% collagen I and 0.5% MaxGel (Sigma-Aldrich E0282) at 50 μl/well for 3 hr at 37° C. Twenty-four hours after plating cultures were switched to a differentiation medium containing DMEM, 2% heat inactivated FBS, 50 μg/ml gentamycin and 10 μg/ml insulin. Forty-eight hours after the switch to differentiation medium, the oligonucleotides were added and cultures were incubated an additional 4 days prior to analysis.

### Cell Staining and High Content Image Analysis

Cultures were washed once with PBS, fixed with a 4% paraformaldehyde solution in PBS for 10 min at room temperature and rinsed once with PBS. For staining, cultures were blocked and permeabilized with 3% bovine serum albumin (BSA) and 0.2% Triton X-100 in PBS for 1 hr at room temperature. Cells were stained with primary antibodies rabbit anti-MyoD (1:100, Fisher Scientific, #NC0819717), mouse anti-Dystrophin MANDRA1 (IgG1(7A10), Santa Cruz, #sc-47760), mouse anti-Dystrophin (IgG2ak, MANDYS106, EMD Millipore, #MABT827), mouse anti-Myosin heavy chain (IgG2B, 1:1000, R&D System, #MAB4470) and secondary antibodies Alexa Fluor 488 donkey anti-rabbit (1:1000, Life Technologies, #A21206), Alexa Fluor 555 goat anti-mouse IgG2a (1:1000, Life Technologies, #A21137), Alexa Fluor 555 goat anti-mouse IgG1 (1:1000, Life Technologies, #A21127) and Alexa Fluor 647 goat anti-mouse IgG2b (1:1000, Life Technologies, #A21242). Nuclei were stained with DAPI (1 μg/ml in PBS) for 20 min at RT prior to imaging. Imaging and analysis were done using a GE InCell 2200 and 6600 instrument and the InCell Investigator software package.

### Results

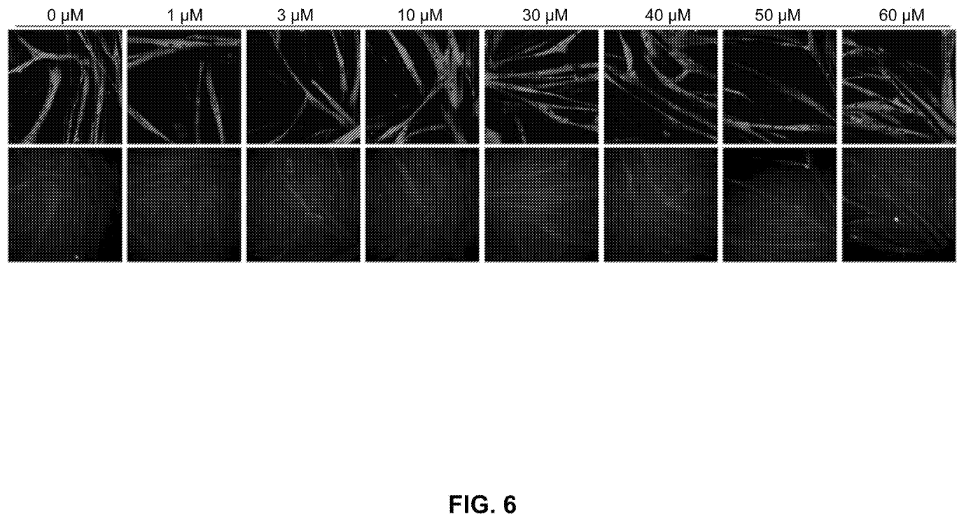

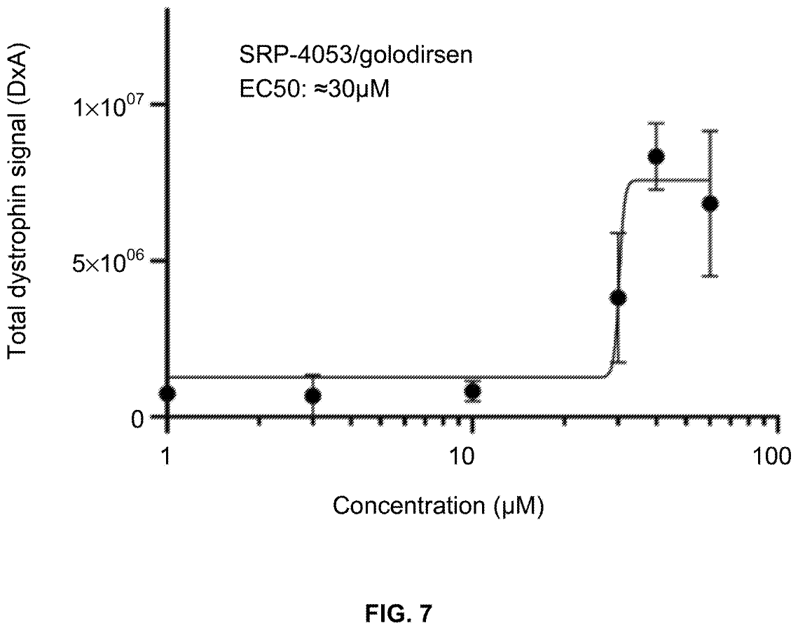

The production of dystrophin protein in the model was evaluated by immunofluorescent staining after four days of continuous exposure to the compound and measured by high content image analysis. Dystrophin protein was detected at concentrations>10 μM SRP-4053/golodirsen (FIG. 6). Emax was achieved at 40 μM allowing for the assignment of an EC50 of approximately 30 μM for dystrophin production in this assay (FIG. 7).

### Example 5. Safety of High-Dose Golodirsen in Non-Human Primates

Golodirsen was administered to male cynomolgus monkeys by IV bolus injection once weekly for 12 weeks, at dose levels of 0 (vehicle), 5, 40, or 320 mg/kg, followed by a 4 week recovery period. A 320 mg/kg dose in humans is predicted to provide AUC and Cₘₐₓ PK that are equivalent to the AUC and Cₘₐₓ PK provided by 320 mg/kg in NHP. Based on the dose-linear exposure relationship observed in NHP, the human 200 mg/kg dose is expected to exhibit similar AUC and Cₘₐₓ PK.

Golodirsen plasma exposures (AUC0-t, Cmax) were consistently high throughout the dosing phase, increased with increasing dose, and there was no evidence of plasma accumulation after 12 weeks of dosing. There were no golodirsen related effects on clinical signs, BW, cardiovascular parameters (including QT intervals), ophthalmology exams, or clinical pathology parameters. Transient increases in Bb and C3a complement fragments were observed and sporadic increases in C5a also occurred, but no consistent pattern was noted, suggesting that golodirsen did not significantly affect the terminal pathway of complement activation. A statistically significant increase in testicular size (testicle weight:BW ratio approximately 50% greater than that of controls) was seen at 320 mg/kg and slightly lower luteinizing hormone concentrations in males at ≥5 mg/kg and follicle stimulating hormone concentrations at ≥40 mg/kg relative to controls but testosterone was not affected. No associated effects on other reproductive endpoints (sperm counts, sperm motility, and morphology) were noted and there were no histopathology correlates for the organ weight change, so these reversible findings were considered non-adverse. The only golodirsen-related histopathology findings noted were minimal diffuse follicular cell hypertrophy in the thyroid gland of 1 animal at 320 mg/kg, which was also considered non-adverse. The NOAEL was considered to be the highest dose level tested, 320 mg/kg (Cmax=1,790 μg/mL, AUC0 t=2,550 μg·hr/mL).

All publications and patent applications cited in this specification are herein incorporated by reference as if each individual publication or patent application were specifically and individually indicated to be incorporated by reference.

1-110. (canceled)

111. A method of treating Duchenne muscular dystrophy (DMD) in a human subject having a mutation of the DMD gene that is amenable to exon 51 skipping, comprising administering to the human subject eteplirsen, or pharmaceutically acceptable salt thereof, wherein eteplirsen, or a pharmaceutically acceptable salt thereof, is administered at a dose of about 80 mg/kg to about 300 mg/kg once a week.

112. The method of claim 111, wherein the dose is about 100 mg/kg.

113. The method of claim 111, wherein the dose is about 200 mg/kg.


SEQUENCE LISTING (54 sequences)

SEQ ID NO: 1 - DNA, 25 bp, Artificial Sequence
  synthetic Antisense oligonucleotide
ctgttccaaatcctgcattgttgcc

SEQ ID NO: 2 - DNA, 24 bp, Artificial Sequence
  synthetic Antisense oligonucleotide
acctccaacatcaaggaagatggc

SEQ ID NO: 3 - DNA, 30 bp, Artificial Sequence
  synthetic Antisense oligonucleotide
gtacctccaacatcaaggaagatggcattt

SEQ ID NO: 4 - DNA, 28 bp, Artificial Sequence
  synthetic Antisense oligonucleotide
gtacctccaacatcaaggaagatggcat

SEQ ID NO: 5 - DNA, 20 bp, Artificial Sequence
  synthetic Antisense oligonucleotide
tcaaggaagatggcatttct

SEQ ID NO: 6 - RNA, 20 nt, Artificial Sequence
  synthetic Antisense oligonucleotide
  misc_feature (8)..(8): methylated Adenosine
  misc_feature (9)..(9): methylated Guanosine
  misc_feature (10)..(10): methylated Adenosine
  misc_feature (12)..(12): methylated Guanosine
  misc_feature (13)..(13): methylated
ucaaggaagauggcauuucu

SEQ ID NO: 7 - DNA, 25 bp, Artificial Sequence
  synthetic Antisense oligonucleotide
gttgcctccggttctgaaggtgttc

SEQ ID NO: 8 - DNA, 25 bp, Artificial Sequence
  synthetic Antisense oligonucleotide
  misc_feature (5)..(5): 5-Methyl Cytosine
  misc_feature (6)..(6): 5-Methyl Cytosine
  misc_feature (8)..(8): 5-Methyl Cytosine
  misc_feature (9)..(9): 5-Methyl Cytosine
  misc_feature (14)..(14): 5-Methyl Cytosine
  misc_feature (25)..(25): 5-Methyl Cytosine
gttgcctccggttctgaaggtgttc

SEQ ID NO: 9 - DNA, 21 bp, Artificial Sequence
  synthetic Antisense oligonucleotide
cctccggttctgaaggtgttc

SEQ ID NO: 10 - DNA, 25 bp, Artificial Sequence
  synthetic Antisense oligonucleotide
ctgaaggtgttcttgtacttcatcc

SEQ ID NO: 11 - DNA, 25 bp, Artificial Sequence
  synthetic Antisense oligonucleotide
cctccggttctgaaggtgttcttgt

SEQ ID NO: 12 - DNA, 28 bp, Artificial Sequence
  synthetic Antisense oligonucleotide
gttgcctccggttctgaaggtgttcttg

SEQ ID NO: 13 - DNA, 30 bp, Artificial Sequence
  synthetic Antisense oligonucleotide
ttgcctccggttctgaaggtgttcttgtac

SEQ ID NO: 14 - DNA, 24 bp, Artificial Sequence
  synthetic Antisense oligonucleotide
ctgttgcctccggttctgaaggtg

SEQ ID NO: 15 - DNA, 31 bp, Artificial Sequence
  synthetic Antisense oligonucleotide
cattcaactgttgcctccggttctgaaggtg

SEQ ID NO: 16 - DNA, 18 bp, Artificial Sequence
  synthetic Antisense oligonucleotide
ctgttgcctccggttctg

SEQ ID NO: 17 - DNA, 22 bp, Artificial Sequence
  synthetic Antisense oligonucleotide
caatgccatcctggagttcctg

SEQ ID NO: 18 - DNA, 34 bp, Artificial Sequence
  synthetic Antisense oligonucleotide
gctgcccaatgccatcctggagttcctgtaagat

SEQ ID NO: 19 - DNA, 28 bp, Artificial Sequence
  synthetic Antisense oligonucleotide
gctgcccaatgccatcctggagttcctg

SEQ ID NO: 20 - DNA, 31 bp, Artificial Sequence
  synthetic Antisense oligonucleotide
gctgcccaatgccatcctggagttcctgtaa

SEQ ID NO: 21 - DNA, 31 bp, Artificial Sequence
  synthetic Antisense oligonucleotide
caatgccatcctggagttcctgtaagatacc

SEQ ID NO: 22 - DNA, 28 bp, Artificial Sequence
  synthetic Antisense oligonucleotide
caatgccatcctggagttcctgtaagat

SEQ ID NO: 23 - DNA, 28 bp, Artificial Sequence
  synthetic Antisense oligonucleotide
tgccatcctggagttcctgtaagatacc

SEQ ID NO: 24 - DNA, 39 bp, Artificial Sequence
  synthetic Antisense oligonucleotide
gctgcccaatgccatcctggagttcctgtaagataccaa

SEQ ID NO: 25 - DNA, 27 bp, Artificial Sequence
  synthetic Antisense oligonucleotide
caatgccatcctggagttcctgtaaga

SEQ ID NO: 26 - DNA, 32 bp, Artificial Sequence
  synthetic Antisense oligonucleotide
gctgcccaatgccatcctggagttcctgtaag

SEQ ID NO: 27 - DNA, 34 bp, Artificial Sequence
  synthetic Antisense oligonucleotide
gcccaatgccatcctggagttcctgtaagatacc

SEQ ID NO: 28 - DNA, 31 bp, Artificial Sequence
  synthetic Antisense oligonucleotide
gcccaatgccatcctggagttcctgtaagat

SEQ ID NO: 29 - DNA, 39 bp, Artificial Sequence
  synthetic Antisense oligonucleotide
ttgccgctgcccaatgccatcctggagttcctgtaagat

SEQ ID NO: 30 - DNA, 28 bp, Artificial Sequence
  synthetic Antisense oligonucleotide
gcccaatgccatcctggagttcctgtaa

SEQ ID NO: 31 - DNA, 34 bp, Artificial Sequence
  synthetic Antisense oligonucleotide
gccgctgcccaatgccatcctggagttcctgtaa

SEQ ID NO: 32 - DNA, 25 bp, Artificial Sequence
  synthetic Antisense oligonucleotide
gcccaatgccatcctggagttcctg

SEQ ID NO: 33 - DNA, 31 bp, Artificial Sequence
  synthetic Antisense oligonucleotide
gccgctgcccaatgccatcctggagttcctg

SEQ ID NO: 34 - DNA, 18 bp, Artificial Sequence
  synthetic Antisense oligonucleotide
  misc_feature (1)..(1): 5-Methyl Cytosine
  misc_feature (3)..(3): 5-Methyl Cytosine
  misc_feature (7)..(7): 5-Methyl Cytosine
  misc_feature (8)..(8): 5-Methyl Cytosine
  misc_feature (13)..(13): 5-Methyl Cytosine
  misc_feature (14)..(14): 5-Methyl Cytosine
  misc_feature (16)..(16): Thymine can be Uracil
  misc_feature (17)..(17): 5-Methyl Cytosine
  misc_feature (18)..(18): 5-Methyl Cytosine
cgctgcccaatgccatcc

SEQ ID NO: 35 - DNA, 25 bp, Artificial Sequence
  synthetic Antisense oligonucleotide
gatctgtcaaatcgcctgcaggtaa

SEQ ID NO: 36 - DNA, 22 bp, Artificial Sequence
  synthetic Antisense oligonucleotide
gatctgtcaaatcgcctgcagg

SEQ ID NO: 37 - DNA, 24 bp, Artificial Sequence
  synthetic Antisense oligonucleotide
cagatctgtcaaatcgcctgcagg

SEQ ID NO: 38 - DNA, 23 bp, Artificial Sequence
  synthetic Antisense oligonucleotide
gatctgtcaaatcgcctgcaggt

SEQ ID NO: 39 - DNA, 21 bp, Artificial Sequence
  synthetic Antisense oligonucleotide
gatctgtcaaatcgcctgcag

SEQ ID NO: 40 - DNA, 25 bp, Artificial Sequence
  synthetic Antisense oligonucleotide
cagatctgtcaaatcgcctgcaggt

SEQ ID NO: 41 - DNA, 23 bp, Artificial Sequence
  synthetic Antisense oligonucleotide
cagatctgtcaaatcgcctgcag

SEQ ID NO: 42 - DNA, 22 bp, Artificial Sequence
  synthetic Antisense oligonucleotide
gggatccagtatacttacaggc

SEQ ID NO: 43 - DNA, 25 bp, Artificial Sequence
  synthetic Antisense oligonucleotide
gggatccagtatacttacaggctcc

SEQ ID NO: 44 - DNA, 23 bp, Artificial Sequence
  synthetic Antisense oligonucleotide
gatccagtatacttacaggctcc

SEQ ID NO: 45 - DNA, 24 bp, Artificial Sequence
  synthetic Antisense oligonucleotide
ggatccagtatacttacaggctcc

SEQ ID NO: 46 - DNA, 26 bp, Artificial Sequence
  synthetic Antisense oligonucleotide
acttcctctttaacagaaaagcatac

SEQ ID NO: 47 - DNA, 22 bp, Artificial Sequence
  synthetic Antisense oligonucleotide
atccagtatacttacaggctcc

SEQ ID NO: 48 - DNA, 25 bp, Artificial Sequence
  synthetic Antisense oligonucleotide
gagctcagatcttctaacttcctct

SEQ ID NO: 49 - DNA, 24 bp, Artificial Sequence
  synthetic Antisense oligonucleotide
gggatccagtatacttacaggctc

SEQ ID NO: 50 - DNA, 27 bp, Artificial Sequence
  synthetic Antisense oligonucleotide
atgggatccagtatacttacaggctcc

SEQ ID NO: 51 - DNA, 25 bp, Artificial Sequence
  synthetic Antisense oligonucleotide
ctgttccaaatcctgcattgttgcc

SEQ ID NO: 52 - RNA, 20 nt, Artificial Sequence
  synthetic Antisense oligonucleotide
ucaaggaagauggcauuucu

SEQ ID NO: 53 - DNA, 25 bp, Artificial Sequence
  synthetic Antisense oligonucleotide
gttgcctccggttctgaaggtgttc

SEQ ID NO: 54 - DNA, 17 bp, Artificial Sequence
  synthetic Antisense oligonucleotide
cgctgcccaatgccacc